(19) United States
(12) Patent Application Publication
(10) Pub. No.: US 2026/0116871 A1
(43) Pub. Date: Apr. 30, 2026

(54) Tyrosine Kinase Inhibitors and Uses Thereof

(71) Applicant: Fujian Haixi Pharmaceuticals Co., Ltd., Fuzhou, Fujian (CN)

(72) Inventors: Guangming CHEN, Fuzhou (CN); Lianchao LIU, Fuzhou (CN); Ruyong WANG, Fuzhou (CN); Fengsen ZHANG, Fuzhou (CN); Xin LIAN, Fuzhou (CN); Nan CHEN, Fuzhou (CN); Zhipeng ZHENG, Fuzhou (CN); Xueyin WU, Fuzhou (CN); Xuan GONG, Fuzhou (CN)

(21) Appl. No.: 19/368,718

(22) Filed: Oct. 24, 2025

(30) Foreign Application Priority Data
Oct. 24, 2024 (CN) 2024114953641

(51) Int. Cl. C07D 409/04; A61K 31/416; A61K 31/4355; A61K 31/4365; A61K 31/437; A61K 31/4436; A61K 31/444; A61K 31/4725; A61K 31/496; A61K 31/4985; A61K 31/506; A61K 31/519; A61K 31/695; C07D 409/06; C07D 409/12; C07D 471/04; C07D 487/04; C07D 491/048; C07D 495/04; C07D 519/00; C07F 7/08

(52) U.S. Cl. CPC C07D 409/04; A61K 31/416; A61K 31/4355; A61K 31/4365; A61K 31/437; A61K 31/4436; A61K 31/444; A61K 31/4725; A61K 31/496; A61K 31/4985; A61K 31/506; A61K 31/519; A61K 31/695; C07D 409/06; C07D 409/12; C07D 471/04; C07D 487/04; C07D 491/048; C07D 495/04; C07D 519/00; C07F 7/0812

(57) ABSTRACT

The present disclosure provides tyrosine kinase inhibitors and uses thereof. Specifically, the present disclosure provides a compound or a pharmaceutically acceptable salt of Formula (I), or a stereoisomer thereof, an isotopologue thereof, a solvate thereof, or a prodrug thereof, wherein each group is as defined elsewhere in the present disclosure.

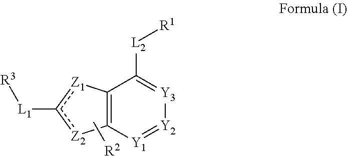

### DECLARATION OF PRIORITY

The present disclosure claims priority to Chinese patent application NO. 2024114953641, filed Oct. 24, 2024, the entire contents of which are incorporated by reference herein in its entirety.

### TECHNICAL FIELD

The present disclosure relates to the field of biomedicine, and specifically to tyrosine kinase inhibitors and uses thereof.

### BACKGROUND

Clinical and preclinical evidence demonstrates that TAMs, as key regulatory immune cells, can promote tumor development. Macrophages primarily exist in two polarized states. Alternatively activated M2 subtype TAMs promote tumor progression by secreting anti-inflammatory cytokines (e.g., interleukin (IL)-10, transforming growth factor-β), whereas activated M1 subtype TAMs enhance immune-mediated tumor killing by producing pro-inflammatory cytokines. To overcome the immunosuppressive and tumor-promoting functions of TAMs, current therapeutic strategies focus on the depletion of TAMs in the tumor microenvironment and the reprogramming of TAMs to promote anti-tumor functions (shifting M2 polarization to M1). The CSF-1R kinase is a product encoded by the proto-oncogene c-fms and belongs to the class III receptor tyrosine kinase family, which also includes FMS-like tyrosine kinase 3 (FLT3), stem cell factor receptor (c-Kit) and PDGFR. CSF-1R binds to its ligands, CSF-1 and IL-34, to activate CSF-1R, thereby playing a crucial role in the proliferation, differentiation, and growth of mononuclear phagocytes. In the tumor microenvironment, a large number of macrophages (TAMs) are present, expressing the CSF-1R on their cell surface. When CSF-1 binds to this receptor, macrophages are converted to a pro-tumor state, driving immune suppression and promoting tumor cell growth. Furthermore, high expression of CSF-1R on tumor cells is associated with lower survival rates in certain cancer patients, indicating tumor dependency, making it a potential therapeutic target. By targeting CSF-1R, the development of CSF-1R/CSF-1 inhibitors can effectively reduce the number of TAMs in tumor tissues, promote the generation of anti-tumor macrophages, and help relieve immune suppression. Additionally, it facilitates the infiltration of various immune cells, including T cells and lymphocytes, into the tumor tissue.

Cancer cell growth and metastasis depend on the formation of neovascularization. Vascular endothelial growth factor (VEGF) is the most potent pro-angiogenic factor because solid tumors depend on angiogenesis to provide oxygen and nutrients to aid in their growth and, in turn, as a pathway for invasion and metastasis. In addition, the VEGFa-VEGFR2 signaling pathway plays an important role in the tumor microenvironment and significantly promotes the proliferation and infiltration of Treg (regulatory T cells) in tumor tissues in animal models. Inhibition of the VEGFa-VEGFR2 signaling pathway can inhibit tumor growth by regulating Tregs, MDSCs, and M2 macrophages. Inhibition of VEGFR2 significantly reduced Treg levels in tumor tissues, further helping to deregulate immunosuppression.

PDGFR is a transmembrane glycoprotein with tyrosine kinase activity, primarily expressed on mesenchymal-derived cells such as fibroblasts, pericytes, vascular smooth muscle cells, and stromal stem/progenitor cells. PDGFR consists of a and 3 subunits. Typically, PDGFRα and PDGFRP exist as monomers in an autoinhibited state. Upon binding with platelet-derived growth factor (PDGF), PDGFR monomers dimerize, forming isomers PDGFR-αα, PDGFR-αβ, and PDGFR-ββ, which mediate a series of downstream signaling responses. The PDGF/PDGFR primarily promotes angiogenesis and vascular maturation, playing a role in growth and development, and is also closely related to the occurrence and progression of various diseases. Due to the characteristics of tumor microvasculature, such as rapid but immature growth, irregularity, and high permeability, the transport and distribution of chemotherapeutic drugs and oxygen to tumor tissues are hindered, making it easy for tumor cells to metastasize and invade other areas through the leaky vessels. The abnormal vascular system creates a malignant tumor microenvironment characterized by hypoxia, low pH, and elevated interstitial fluid pressure, which interferes with the function of immune cells within the tumor and reduces the efficacy of radiation therapy and chemotherapy. Therefore, the focus of current anti-angiogenic therapy has shifted from simply destroying blood vessels to “normalizing” tumor microvasculature, in order to increase oxygen supply and alter the direction of drug delivery. The root cause of abnormal vascular growth in tumor tissues lies in the disruption of various growth factors within the microenvironment. The PDGF/PDGFR pathway, as a key factor in regulating vascular growth and recruiting pericytes to promote vascular maturation, plays an indispensable role in this process.

Discoidin domain receptor 1 (DDR1) is a novel receptor tyrosine kinase (RTK) that, upon activation, regulates matrix metalloproteinase (MMP), epithelial mesenchymal transition (EMT), and plays an important role in tumor cell migration. Some studies have shown that inhibition of DDR1 expression can induce autophagy and improve the sensitivity of tumor cells to radiation therapy and chemotherapy.

As is well known, multi-target drugs have obvious advantages over combination drugs and multi-component drugs: due to single component, they are better than combination drugs and multi-component drugs in drug metabolism; they overcome the adverse reactions caused by interactions between components; they are convenient to administer, and there is no problem of dosage or ratio in combination drugs; they have predictable pharmacodynamics (PD) and pharmacokinetic (PK) properties; under the premise of the same therapeutic effect, due to synergistic effect can make the drug dose lower, thus improving the adverse effects of highly selective single-target drugs; slow down the occurrence of drug resistance.

It is an object of the present disclosure to provide a compound having tyrosine kinase inhibitory activity, which achieves enhanced tumor suppression efficacy through synergistic effects of multi-target inhibition and reduces the occurrence of drug resistance.

### SUMMARY OF THE INVENTION

An object of the present disclosure is to provide a compound of Formula (I), or a pharmaceutically acceptable salt thereof, stereoisomer thereof, isotopologue thereof, or prodrug thereof.

Another object of the present disclosure is to provide a use of the above-mentioned compound of Formula (I), or a pharmaceutically acceptable salt thereof, stereoisomer thereof, isotopologue thereof, or prodrug thereof.

An aspect of the present disclosure relates to a compound of Formula (I), or a pharmaceutically acceptable salt thereof, stereoisomer thereof, isotopologue thereof, solvate thereof, or prodrug thereof,

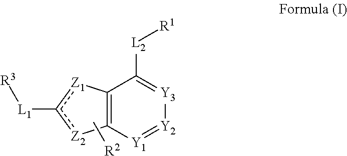

wherein the dashed line represents the absence or presence of a bond, with the proviso that the two dashed lines are not simultaneously absent nor simultaneously bonds;
Z₁ represents S, O or C;
Z₂ represents S, O or C;
Z₁ and Z₂ are not both C;
L₁ represents absent, —(CR^{a}R^{b})ₒ—, —O—, —S—, —S(O)—, —S(O)₂—, —C(O)— or —NR^{a};
L₂ represents —(CR^{a}R^{b})ₒ—, —CR^{a}R^{b}O—, —OCR^{a}R^{b}—, —O—, —C(O)—, —NR^{a}—, —S(O)₂NR^{a}—, —OC(O)—, —C(O)O—, —NR^{a}S(O)₂—, —CR^{a}R^{b}NR^{a}—, —NR^{a}CR^{a}R—, —NR^{a}C(O)NR—, —NR^{a}C(O)NR^{b}CR^{a}R—, —NR^{a}C(O)O—, —NR^{a}C(O)— or —C(O)NR^{a}—;
o at each occurrence independently represents an integer from 1 to 20;
Y₁ represents CR^{Y1} or N;
Y₂ represents CR^{Y2} or N;
Y₃ represents CR^{Y3} or N;
R¹ represents substituted or unsubstituted C₁-C₆ alkyl, substituted or unsubstituted C₃-C₁₀ cycloalkyl, substituted or unsubstituted C₆-C₁₀ cycloalkenyl, substituted or unsubstituted 4-10 membered heterocycloalkyl, substituted or unsubstituted 6-10 membered heterocycloalkenyl, substituted or unsubstituted C₆-C₁₀ aryl, or substituted or unsubstituted 5-10 membered heteroaryl;
R² represents hydrogen, deuterium, halogen, hydroxy, cyano, —NR^{a}R^{b}, substituted or unsubstituted C₁-C₆ alkyl, substituted or unsubstituted C₁-C₆ alkoxy, substituted or unsubstituted C₃-C₁₀ cycloalkyl, substituted or unsubstituted C₂-C₆ alkenyl, or substituted or unsubstituted C₂-C₆ alkynyl;
R³ represents optionally substituted or unsubstituted heteroaryl;
wherein, R^{Y1}, R^{Y2} and R^{Y3} each independently represent hydrogen, deuterium, halogen, cyano, nitro, substituted or unsubstituted C₁-C₆ alkyl, substituted or unsubstituted C₁-C₆ alkoxy, substituted or unsubstituted C₂-C₆ alkenyl, substituted or unsubstituted C₂-C₆ alkynyl, —OR^{a}, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, substituted or unsubstituted C₃-C₆ cycloalkyl, substituted or unsubstituted C₃-C₆ heterocycloalkyl, substituted or unsubstituted C₆-C₁₀ aryl, or substituted or unsubstituted 5-10 membered heteroaryl;
wherein R^{a} and R^{b} each independently represent hydrogen, deuterium, halogen, cyano, hydroxy, amino, substituted or unsubstituted C₁-C₆ alkyl, substituted or unsubstituted C₁-C₆ alkoxy, or substituted or unsubstituted C₃-C₁₀ cycloalkyl; or R^{a} and R^{b} together with the atom which they are attached to form a 3-6 membered ring, and the said ring optionally contains 0, 1, or 2 heteroatoms selected from O, N, S, or P, and further, the said ring may optionally substituted by substituents selected from halogen, hydroxy, or C₁-C₆ alkyl.

The aforementioned substituents can be any substituents, preferably hydrogen, deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, —Si(R^{a})₃, C₃-C₆ cycloalkyl, C₃-C₆ heterocycloalkyl, C₆-C₁₀ aryl, 5-10 membered heteroaryl, or C₁-C₆ alkyl, C₁-C₆ alkoxy, C₂-C₆ alkenyl, C₂-C₆ alkynyl, C₃-C₆ cycloalkyl, C₃-C₆ heterocycloalkyl, C₆-C₁₀ aryl, 5-10 membered heteroaryl, each of which is optionally substituted.

In some preferred embodiments, when R¹ is substituted C₃-C₁₀ cycloalkyl, C₆-C₁₀ cycloalkenyl, 4-10 membered heterocycloalkyl, 6-10 membered heterocycloalkenyl, C₆-C₁₀ aryl, or 5-10 membered heteroaryl, two adjacent substituents on the ring, together with the atoms to which they are attached, can form a 3-6 membered ring.

In some preferred embodiments, when R³ is substituted, two adjacent substituents on the R³ ring, together with the atoms to which they are attached, can form a 3-6 membered ring, and the said ring may optionally contain 0, 1, or 2 heteroatoms selected from O, N, and S.

In some preferred embodiments, the compound has a structure represented by Formula (I),

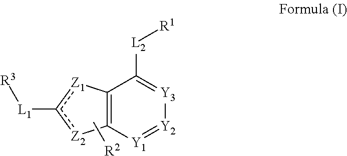

wherein the dashed line represents the absence or presence of a bond, with the proviso that the two dashed lines are not simultaneously absent nor simultaneously bonds;
Z₁ represents S, O or C;
Z₂ represents S, O or C;
Z₁ and Z₂ are not both C;
L₁ represents absent, —(CR^{a}R^{b})ₒ—, —O—, —S—, —S(O)—, —S(O)₂—, —C(O)— or —NR^{a};
L₂ represents —(CR^{a}R^{b})ₒ—, —CR^{a}R^{b}O—, —OCR^{a}R^{b}—, —O—, —C(O)—, —NR^{a}—, —S(O)₂NR^{a}—, —OC(O)—, —C(O)O—, —NR^{a}S(O)₂—, —CR^{a}R^{b}NR^{a}—, —NR^{a}CR^{a}R—, —NR^{a}C(O)NR—, —NR^{a}C(O)NR^{b}CR^{a}R—, —NR^{a}C(O)O—, —NR^{a}C(O)— or —C(O)NR^{a}—;
o at each occurrence independently represents an integer from 1 to 20;
Y₁ represents CR^{Y1} or N;
Y₂ represents CR^{Y2} or N;
Y₃ represents CR^{Y3} or N;
R¹ represents C₁-C₆ alkyl, C₃-C₁₀ cycloalkyl, C₆-C₁₀ cycloalkenyl, 4-10 membered heterocycloalkyl, 6-10 membered heterocycloalkenyl, C₆-C₁₀ aryl, or 5-10 membered heteroaryl; and the C₁-C₆ alkyl, C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl are further optionally substituted with at least one substituent independently selected from hydrogen, deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ heterocycloalkyl, C₆-C₁₀ aryl, 5-10 membered heteroaryl, C₁-C₆ alkyl substituted with R¹¹, C₁-C₆ alkoxy substituted with R¹¹, C₂-C₆ alkenyl substituted with R¹¹, C₂-C₆ alkynyl substituted with R¹¹, C₃-C₆ cycloalkyl substituted with R¹¹, C₃-C₆ heterocycloalkyl substituted with R¹¹, C₆-C₁₀ aryl substituted with R¹¹, and 5-10 membered heteroaryl substituted with R¹¹; wherein R¹¹ at each occurrence is independently deuterium, halogen, cyano, nitro, C₁-C₆ alkyl, C₁-C₆ alkyl substituted with R¹², C₁-C₆ alkoxy, C₁-C₆ alkoxy substituted with R¹², C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ cycloalkyl substituted with R¹², C₃-C₆ heterocycloalkyl, C₃-C₆ heterocycloalkyl substituted with R¹², C₆-C₁₀ aryl, C₆-C₁₀ aryl substituted with R¹², 5-10 membered heteroaryl, or 5-10 membered heteroaryl substituted with R¹²; R¹² at each occurrence is independently deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, and C₃-C₆ cycloalkyl;
R² represents hydrogen, deuterium, halogen, hydroxy, amino, cyano, C₂-C₆ alkenyl, C₂-C₆ alkynyl, C₁-C₆ alkyl, C₁-C₆ alkoxy, or C₃-C₁₀ cycloalkyl; and the C₁-C₆ alkyl, C₃-C₁₀ cycloalkyl are further optionally substituted with at least one substituent independently selected from deuterium, halogen, hydroxy, C₁-C₆ alkyl, amino, cyano, C₂-C₆ alkenyl, C₂-C₆ alkynyl, and C₃-C₁₀ cycloalkyl.
R³ represents optionally substituted or unsubstituted heteroaryl;
wherein, R^{Y1}, R^{Y2} and R^{Y3} each independently represent hydrogen, deuterium, halogen, cyano, nitro, C₁-C₆ alkyl, C₁-C₆ alkoxy, C₂-C₆ alkenyl, C₂-C₆ alkynyl, —OR^{a}, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ heterocycloalkyl, C₆-C₁₀ aryl, 5-10 membered heteroaryl; and the C₁-C₆ alkyl, C₁-C₆ alkoxy, C₂-C₆ alkenyl, C₂-C₆ alkynyl, C₃-C₆ cycloalkyl, C₃-C₆ heterocycloalkyl, C₆-C₁₀ aryl, 5-10 membered heteroaryl are further optionally substituted by at least one substituent independently selected from hydrogen, deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ heterocycloalkyl, C₆-C₁₀ aryl, and 5-10 membered heteroaryl.
wherein R^{a} and R each independently represent hydrogen, deuterium, halogen, cyano, hydroxy, amino, C₁-C₆ alkyl, C₁-C₆ haloalkyl, hydroxy-substituted C₁-C₆ alkyl, C₃-C₁₀ cycloalkyl, C₃-C₁₀ halocycloalkyl, or hydroxy-substituted C₃-C₁₀ cycloalkyl; or R^{a} and R^{b}, together with the atom to which they are attached, form a 3-6 membered ring, and the said ring optionally contain 0, 1, or 2 heteroatoms selected from O, N, S, or P, and further, the said ring may optionally substituted by substituent selected from halogen, hydroxy, and C₁-C₆ alkyl.

In some preferred embodiments, Z₁ and Z₂ are not both S or O.

In some preferred embodiments, Z₁ represents S or O, and Z₂ represents C.

In some preferred embodiments, Z₁ represents C, and Z₂ represents S or O.

In some preferred embodiments, Z₁ represents C, and Z₂ represents S.

In some preferred embodiments, Z₁ represents S, and Z₂ represents C.

In some preferred embodiments, the compound has a structure represented by Formula (II),

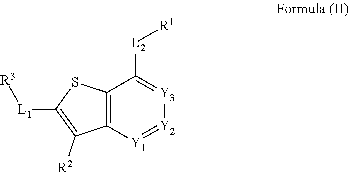

Wherein, L₁ represents absent, —(CR^{a}R^{b})ₒ—, —O—, —S—, —S(O)—, —S(O)₂—, —C(O)— or —NR^{a}—;
L₂ represents —(CR^{a}R^{b})ₒ—, —CR^{a}R^{b}O—, —OCR^{a}R—, —O—, —C(O)—, —NR^{a}—, —S(O)₂NR^{a}—, —OC(O)—, —C(O)O—, —NR^{a}S(O)₂—, —CR^{a}R^{b}NR^{a}—, —NR^{a}CR^{a}R—, —NR^{a}C(O)NR—, —NR^{a}C(O)NR^{b}CR^{a}R—, —NR^{a}C(O)O—, —NR^{a}C(O)— or —C(O)NR^{a}—;
o at each occurrence independently represents an integer from 1 to 20;
Y₁ represents CR^{Y1} or N;
Y₂ represents CR^{Y2} or N;
Y₃ represents CR^{Y3} or N;
R¹ represents substituted or unsubstituted C₁-C₆ alkyl, substituted or unsubstituted C₃-C₁₀ cycloalkyl, substituted or unsubstituted C₆-C₁₀ cycloalkenyl, substituted or unsubstituted 4-10 membered heterocycloalkyl, substituted or unsubstituted 6-10 membered heterocycloalkenyl, substituted or unsubstituted C₆-C₁₀ aryl, or substituted or unsubstituted 5-10 membered heteroaryl;
R² represents hydrogen, deuterium, halogen, hydroxy, cyano, —NR^{a}R^{b}, substituted or unsubstituted C₁-C₆ alkyl, substituted or unsubstituted C₁-C₆ alkoxy, substituted or unsubstituted C₃-C₁₀ cycloalkyl, substituted or unsubstituted C₂-C₆ alkenyl, or substituted or unsubstituted C₂-C₆ alkynyl;
R³ represents optionally substituted or unsubstituted heteroaryl;
wherein, R^{Y1}, R^{Y2} and R^{Y3} each independently represent hydrogen, deuterium, halogen, cyano, nitro, substituted or unsubstituted C₁-C₆ alkyl, substituted or unsubstituted C₁-C₆ alkoxy, substituted or unsubstituted C₂-C₆ alkenyl, substituted or unsubstituted C₂-C₆ alkynyl, —OR^{a}, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, substituted or unsubstituted C₃-C₆ cycloalkyl, substituted or unsubstituted C₃-C₆ heterocycloalkyl, substituted or unsubstituted C₆-C₁₀ aryl, or substituted or unsubstituted 5-10 membered heteroaryl;
wherein R^{a} and R^{b} each independently represent hydrogen, deuterium, halogen, cyano, hydroxy, amino, substituted or unsubstituted C₁-C₆ alkyl, substituted or unsubstituted C₁-C₆ alkoxy, or substituted or unsubstituted C₃-C₁₀ cycloalkyl; or R^{a} and R^{b} together with the atom which they are attached to form a 3-6 membered ring, and the said ring optionally contains 0, 1, or 2 heteroatoms selected from O, N, S, or P, and further, the said ring may optionally substituted by substituents selected from halogen, hydroxy, or C₁-C₆ alkyl.

In some preferred embodiments, when R¹ is substituted C₃-C₁₀ cycloalkyl, C₆-C₁₀ cycloalkenyl, 4-10 membered heterocycloalkyl, 6-10 membered heterocycloalkenyl, C₆-C₁₀ aryl, or 5-10 membered heteroaryl, two adjacent substituents on the ring, together with the atoms to which they are attached, can form a 3-6 membered ring; wherein said ring optionally contains 0, 1, or 2 heteroatoms selected from O, N, and S.

In some preferred embodiments, when R³ is substituted, two adjacent substituents on the R³ ring, together with the atoms to which they are attached, can form a 3-6 membered ring; wherein said ring optionally contains 0, 1, or 2 heteroatoms selected from O, N, and S.

In some preferred embodiments, the compound has a structure represented by Formula (II),

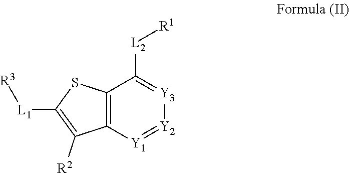

Wherein, L₁ represents absent, —(CR^{a}R^{b})ₒ—, —O—, —S—, —S(O)—, —S(O)₂—, —C(O)— or —NR^{a};
L₂ represents —(CR^{a}R^{b})ₒ—, —CR^{a}R^{b}O—, —OCR^{a}R^{b}—, —O—, —C(O)—, —NR^{a}—, —S(O)₂NR^{a}—, —OC(O)—, —C(O)O—, —NR^{a}S(O)₂—, —CR^{a}R^{b}NR^{a}—, —NR^{a}CR^{a}R—, —NR^{a}C(O)NR—, —NR^{a}C(O)NR^{b}CR^{a}R—, —NR^{a}C(O)O—, —NR^{a}C(O)— or —C(O)NR^{a}—;
o at each occurrence independently represents an integer from 1 to 20;
Y₁ represents CR^{Y1} or N;
Y₂ represents CR^{Y2} or N;
Y₃ represents CR^{Y3} or N;
R¹ represents C₁-C₆ alkyl, C₃-C₁₀ cycloalkyl, C₆-C₁₀ cycloalkenyl, 4-10 membered heterocycloalkyl, 6-10 membered heterocycloalkenyl, C₆-C₁₀ aryl, or 5-10 membered heteroaryl; and the C₁-C₆ alkyl, C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl are further optionally substituted with at least one substituent independently selected from hydrogen, deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ heterocycloalkyl, C₆-C₁₀ aryl, 5-10 membered heteroaryl, C₁-C₆ alkyl substituted with R¹¹, C₁-C₆ alkoxy substituted with R¹¹, C₂-C₆ alkenyl substituted with R¹¹, C₂-C₆ alkynyl substituted with R¹¹, C₃-C₆ cycloalkyl substituted with R¹¹, C₃-C₆ heterocycloalkyl substituted with R¹¹, C₆-C₁₀ aryl substituted with R¹¹, and 5-10 membered heteroaryl substituted with R¹¹;
wherein R¹¹ at each occurrence is independently deuterium, halogen, cyano, nitro, C₁-C₆ alkyl, C₁-C₆ alkyl substituted with R¹², C₁-C₆ alkoxy, C₁-C₆ alkoxy substituted with R¹², C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ cycloalkyl substituted with R¹², C₃-C₆ heterocycloalkyl, C₃-C₆ heterocycloalkyl substituted with R¹², C₆-C₁₀ aryl, C₆-C₁₀ aryl substituted with R¹², 5-10 membered heteroaryl, or 5-10 membered heteroaryl substituted with R¹²;
R¹² at each occurrence is independently selected from deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, and C₃-C₆ cycloalkyl;
R² represents hydrogen, deuterium, halogen, hydroxy, amino, cyano, C₂-C₆ alkenyl, C₂-C₆ alkynyl, C₁-C₆ alkyl, or C₃-C₁₀ cycloalkyl; and the C₁-C₆ alkyl, C₃-C₁₀ cycloalkyl are further optionally substituted with at least one substituent independently selected from deuterium, halogen, hydroxy, C₁-C₆ alkyl, and C₃-C₁₀ cycloalkyl;
R³ represents optionally substituted or unsubstituted heteroaryl;
wherein, R^{Y1}, R^{Y2} and R^{Y3} each independently represent hydrogen, deuterium, halogen, cyano, nitro, C₁-C₆ alkyl, C₁-C₆ alkoxy, C₂-C₆ alkenyl, C₂-C₆ alkynyl, —OR^{a}, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ heterocycloalkyl, C₆-C₁₀ aryl or 5-10 membered heteroaryl; and the C₁-C₆ alkyl, C₁-C₆ alkoxy, C₂-C₆ alkenyl, C₂-C₆ alkynyl, C₃-C₆ cycloalkyl, C₃-C₆ heterocycloalkyl, C₆-C₁₀ aryl, 5-10 membered heteroaryl are further optionally substituted by at least one substituent independently selected from hydrogen, deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ heterocycloalkyl, C₆-C₁₀ aryl, and 5-10 membered heteroaryl;
wherein R^{a} and R^{b} each independently represent hydrogen, deuterium, halogen, cyano, hydroxy, amino, C₁-C₆ alkyl, C₁-C₆ haloalkyl, hydroxy-substituted C₁-C₆ alkyl, C₃-C₁₀ cycloalkyl, C₃-C₁₀ halocycloalkyl, or hydroxy-substituted C₃-C₁₀ cycloalkyl; or R^{a} and R^{b}, together with the atom to which they are attached, form a 3-6 membered ring, and the said ring optionally contain 0, 1, or 2 heteroatoms selected from O, N, S, or P, and further, the said ring may optionally substituted by substituent selected from halogen, hydroxy, and C₁-C₆ alkyl.

In some preferred embodiments, L₂ represents —S(O)₂NR^{a}—, —C(O)O—, —NR^{a}S(O)₂—, —NR^{a}C(O)NR^{b}—, —NR^{a}C(O)NR^{b}CR^{a}R^{b}—, —NR^{a}C(O)O—, —NR^{a}C(O)— or —C(O)NR^{a}—; more preferably, L₂ represents —C(O)NR^{a}—, —NR^{a}C(O)— or —NR^{a}C(O)NR—; more preferably, L₂ represents —C(O)NR^{a}— or —NR^{a}C(O)—; more preferably, L₂ represents —C(O)NR^{a}—.

In some preferred embodiments, L₂ represents —S(O)₂NR^{a}—, —C(O)O—, —NR^{a}S(O)₂—, —NR^{a}C(O)NR^{b}—, —NR^{a}C(O)NR^{b}CR^{a}R^{b}—, —NR^{a}C(O)O—, —NR^{a}C(O)— or —C(O)NR^{a}—; more preferably, L₂ represents —C(O)NR^{a}—, —NR^{a}C(O)— or —NR^{a}C(O)NR—; more preferably, L₂ represents —C(O)NR^{a}— or —NR^{a}C(O)—; more preferably, L₂ represents —C(O)NR^{a}—, wherein R^{a} and R^{b} each independently represent hydrogen, deuterium, fluorine, chlorine, bromine, iodine, methyl, ethyl, n-propyl, isopropyl, fluoromethyl, fluoroethyl, —CH₂OH, —CH₂CH₂OH, —CH(OH)CH₃ or cyclopropyl.

In some preferred embodiments, L₂ represents —C(O)NH—, —C(O)N(CH₃)—, —N(CH₃)C(O)—, —NHC(O)— or —NHC(O)NH—.

In some preferred embodiments, L₂ represents —C(O)NH—, —C(O)N(CH₃)—, —NHC(O)— or —N(CH₃)C(O)—.

In some preferred embodiments, L₂ represents —C(O)NH— or —C(O)N(CH₃)—.

In some preferred embodiments, L₂ represents —C(O)NH—.

In some preferred embodiments, L₁ represents absent, —O—, —CR^{a}R^{b}—, —NR^{a}—, —C(O)—; wherein R^{a} and R^{b} each independently represent hydrogen, deuterium, fluorine, chlorine, bromine, iodine, hydroxy, amino, cyano, methyl, ethyl, n-propyl, isopropyl, fluoromethyl, fluoroethyl, —CH₂OH, —CH₂CH₂OH, —CH(OH)CH₃ or cyclopropyl.

In some preferred embodiments, L₁ represents absent, —O—, —CR^{a}R^{b}—, —NR^{a}—; wherein R^{a} and R^{b} each independently represent hydrogen, deuterium, fluorine, chlorine, bromine, iodine, hydroxy, amino, cyano, methyl, ethyl, n-propyl, isopropyl, fluoromethyl, fluoroethyl, —CH₂OH, —CH₂CH₂OH, —CH(OH)CH₃ or cyclopropyl.

In some preferred embodiments, L₁ represents absent, —O—, —CH₂—, —CF₂—, —NH—, —C(O)—.

In some preferred embodiments, L₁ represents absent, —O—, —CH₂— or —NH—; More preferably, L₁ represents absent, —O—; More preferably, L₁ represents absent.

In some preferred embodiments, the compound has a structure represented by Formula (III),

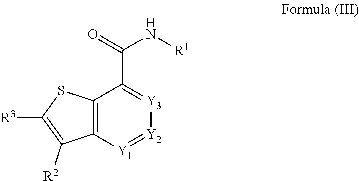

Wherein, Y₁ represents CR^{Y1} or N;
Y₂ represents CR^{Y2} or N;
Y₃ represents CR^{Y3} or N;
R¹ represents substituted or unsubstituted C₁-C₆ alkyl, substituted or unsubstituted C₃-C₁₀ cycloalkyl, substituted or unsubstituted C₆-C₁₀ cycloalkenyl, substituted or unsubstituted 4-10 membered heterocycloalkyl, substituted or unsubstituted 6-10 membered heterocycloalkenyl, substituted or unsubstituted C₆-C₁₀ aryl, or substituted or unsubstituted 5-10 membered heteroaryl;
R² represents hydrogen, deuterium, halogen, hydroxy, cyano, —NR^{a}R^{b}, substituted or unsubstituted C₁-C₆ alkyl, substituted or unsubstituted C₁-C₆ alkoxy, substituted or unsubstituted C₃-C₁₀ cycloalkyl, substituted or unsubstituted C₂-C₆ alkenyl, or substituted or unsubstituted C₂-C₆ alkynyl;
R³ represents optionally substituted or unsubstituted heteroaryl;
wherein R^{Y1}, R^{Y2}, and R^{Y3} each independently represent hydrogen, deuterium, halogen, cyano, nitro, substituted or unsubstituted C₁-C₆ alkyl, substituted or unsubstituted C₁-C₆ alkoxy, substituted or unsubstituted C₂-C₆ alkenyl, substituted or unsubstituted C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, substituted or unsubstituted C₃-C₆ cycloalkyl, substituted or unsubstituted C₃-C₆ heterocycloalkyl, substituted or unsubstituted C₆-C₁₀ aryl, or substituted or unsubstituted 5-10 membered heteroaryl;
wherein R^{a} and R^{b} each independently represent hydrogen, deuterium, halogen, cyano, hydroxy, amino, substituted or unsubstituted C₁-C₆ alkyl, substituted or unsubstituted C₃-C₁₀ cycloalkyl; or R^{a} and R^{b}, together with the atom to which they are attached, form a 3-6 membered ring; wherein said ring optionally contains 1 or 2 heteroatoms selected from O, N, S, or P; and further wherein said ring may be optionally substituted by substituent selected from halogen, hydroxy, and C₁-C₆ alkyl.

In some preferred embodiments, when R¹ is substituted C₃-C₁₀ cycloalkyl, C₆-C₁₀ cycloalkenyl, 4-10 membered heterocycloalkyl, 6-10 membered heterocycloalkenyl, C₆-C₁₀ aryl, or 5-10 membered heteroaryl, two adjacent substituents on the ring, together with the atoms to which they are attached, can form a 3-6 membered ring.

In some preferred embodiments, when R³ is substituted, two adjacent substituents on the R³ ring, together with the atoms to which they are attached, can form a 3-6 membered ring; wherein said ring optionally contains 0, 1, or 2 heteroatoms selected from O, N, S, or P.

In some preferred embodiments, the compound has a structure represented by Formula (III),

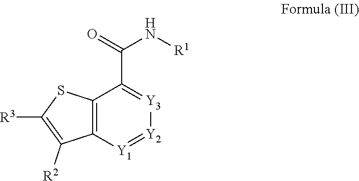

Wherein, Y₁ represents CR^{Y1} or N;
Y₂ represents CR^{Y2} or N;
Y₃ represents CR^{Y3} or N;
R¹ represents C₁-C₆ alkyl, C₃-C₁₀ cycloalkyl, C₆-C₁₀ cycloalkenyl, 4-10 membered heterocycloalkyl, 6-10 membered heterocycloalkenyl, C₆-C₁₀ aryl, or 5-10 membered heteroaryl; and the C₁-C₆ alkyl, C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl are further optionally substituted with at least one substituent independently selected from hydrogen, deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ heterocycloalkyl, C₆-C₁₀ aryl, 5-10 membered heteroaryl, C₁-C₆ alkyl substituted with R¹¹, C₁-C₆ alkoxy substituted with R¹¹, C₂-C₆ alkenyl substituted with R¹¹, C₂-C₆ alkynyl substituted with R¹¹, C₃-C₆ cycloalkyl substituted with R¹¹, C₃-C₆ heterocycloalkyl substituted with R¹¹, C₆-C₁₀ aryl substituted with R¹¹, and 5-10 membered heteroaryl substituted with R¹¹;
wherein R¹¹ at each occurrence is independently deuterium, halogen, cyano, nitro, C₁-C₆ alkyl, C₁-C₆ alkyl substituted with R¹², C₁-C₆ alkoxy, C₁-C₆ alkoxy substituted with R¹², C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ cycloalkyl substituted with R¹², C₃-C₆ heterocycloalkyl, C₃-C₆ heterocycloalkyl substituted with R¹², C₆-C₁₀ aryl, C₆-C₁₀ aryl substituted with R¹², 5-10 membered heteroaryl, or 5-10 membered heteroaryl substituted with R¹²;
R¹² at each occurrence is independently selected from deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, and C₃-C₆ cycloalkyl;
R² represents hydrogen, deuterium, halogen, hydroxy, amino, cyano, C₂-C₆ alkenyl, C₂-C₆ alkynyl, C₁-C₆ alkyl, or C₃-C₁₀ cycloalkyl; and the C₁-C₆ alkyl, C₃-C₁₀ cycloalkyl are further optionally substituted by at least one substituent independently selected from deuterium, halogen, hydroxy, C₁-C₆ alkyl, and C₃-C₁₀ cycloalkyl;
R³ represents optionally substituted or unsubstituted heteroaryl;
wherein, R^{Y1}, R^{Y2} and R^{Y3} each independently represent hydrogen, deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ heterocycloalkyl, C₆-C₁₀ aryl or 5-10 membered heteroaryl; and the C₁-C₆ alkyl, C₁-C₆ alkoxy, C₂-C₆ alkenyl, C₂-C₆ alkynyl, C₃-C₆ cycloalkyl, C₃-C₆ heterocycloalkyl, C₆-C₁₀ aryl, 5-10 membered heteroaryl are further optionally substituted by at least one substituent independently selected from hydrogen, deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ heterocycloalkyl, C₆-C₁₀ aryl, and 5-10 membered heteroaryl;
wherein R^{a} and R^{b} each independently represent hydrogen, deuterium, halogen, cyano, hydroxy, amino, C₁-C₆ alkyl, C₁-C₆ haloalkyl, hydroxy-substituted C₁-C₆ alkyl, C₃-C₁₀ cycloalkyl, C₃-C₁₀ halocycloalkyl, or hydroxy-substituted C₃-C₁₀ cycloalkyl; or R^{a} and R^{b}, together with the atom to which they are attached, form a 3-6 membered ring, and the said ring optionally contain 1 or 2 heteroatoms selected from O, N, S, or P, and further, the said ring may optionally substituted by substituent selected from halogen, hydroxy, and C₁-C₆ alkyl.

In some preferred embodiments, when R¹ is substituted C₃-C₁₀ cycloalkyl, C₆-C₁₀ cycloalkenyl, 4-10 membered heterocycloalkyl, 6-10 membered heterocycloalkenyl, C₆-C₁₀ aryl, or 5-10 membered heteroaryl, two adjacent substituents on the ring may, together with the atoms to which they are attached, form a 3-6 membered ring, which optionally contains 0, 1, or 2 heteroatoms selected from O, N, and S.

In some preferred embodiments, when R³ is substituted, two adjacent substituents on the R³ ring may, together with the atoms to which they are attached, form a 3-6 membered ring, and the said ring optionally contains 0, 1, or 2 heteroatoms selected from O, N, and S.

In some preferred embodiments, Y₁ represents CR^{Y1} or N; Y₂ represents CR¹² or N; Y₃ represents CR^{Y3} or N; and Y₁, Y₂, and Y₃ are not all N at the same time.

In some preferred embodiments, Y₁ represents CR^{Y1}; Y₂ represents CR^{Y2}; Y₃ represents CR^{Y3}; or at least one of Y₁, Y₂, and Y₃ is N.

In some preferred embodiments, Y₁ represents CR^{Y1}, Y₂ represents CR^{Y2}, Y₃ represents CR^{Y3}; or Y₁ represents CR^{Y1}, Y₂ represents CR^{Y2}, Y₃ represents N.

In some preferred embodiments, Y₁ represents CR^{Y1}, Y₂ represents CR^{Y2}, Y₃ represents CR^{Y3}; or Y₁ represents CR^{Y1}, Y₂ represents N, Y₃ represents CR^{Y3}.

In some preferred embodiments, Y₁ represents CR^{Y1}, Y₂ represents CR^{Y2}, Y₃ represents CR^{Y3}; or Y₁ represents N, Y₂ represents CR^{Y2}, Y₃ represents CR^{Y3}.

In some preferred embodiments, Y₁ represents CR^{Y1}, Y₂ represents CR^{Y2}, Y₃ represents CR^{Y3}.

In some preferred embodiments, R^{Y1}, R^{Y2} and R^{Y3} each independently represent hydrogen, deuterium, fluorine, chlorine, bromine, iodine, amino, hydroxy, cyano, methyl, ethyl, n-propyl, isopropyl, n-butyl, isobutyl, sec-butyl, tert-butyl, methoxy, ethoxy, fluoromethyl, fluoroethyl, cyclopropyl, cyclobutyl, cyclopentyl, or cyclohexyl. Preferably, R^{Y1}, R^{Y2} and R^{Y3} each independently represent hydrogen, deuterium, fluorine, chlorine, bromine, iodine, fluoromethyl, or methoxy.

In some preferred embodiments, R¹ represents C₁-C₆ alkyl, C₃-C₁₀ cycloalkyl, C₆-C₁₀ cycloalkenyl, 4-10 membered heterocycloalkyl, 6-10 membered heterocycloalkenyl, C₆-C₁₀ aryl, or 5-10 membered heteroaryl; and the C₁-C₆ alkyl, C₃-C₁₀ cycloalkyl, C₆-C₁₀ cycloalkenyl, 4-10 membered heterocycloalkyl, 6-10 membered heterocycloalkenyl, C₆-C₁₀ aryl, 5-10 membered heteroaryl are further optionally substituted with at least one substituent independently selected from hydrogen, deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ heterocycloalkyl, C₆-C₁₀ aryl, 5-10 membered heteroaryl, C₁-C₆ alkyl substituted with R¹¹, C₁-C₆ alkoxy substituted with R¹¹, C₂-C₆ alkenyl substituted with R¹¹, C₂-C₆ alkynyl substituted with R¹¹, C₃-C₆ cycloalkyl substituted with R¹¹, C₃-C₆ heterocycloalkyl substituted with R¹¹, C₆-C₁₀ aryl substituted with R¹¹, and 5-10 membered heteroaryl substituted with R¹¹; wherein R¹¹ at each occurrence is independently deuterium, halogen, cyano, nitro, C₁-C₆ alkyl, C₁-C₆ alkyl substituted with R¹², C₁-C₆ alkoxy, C₁-C₆ alkoxy substituted with R¹², C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ cycloalkyl substituted with R¹², C₃-C₆ heterocycloalkyl, C₃-C₆ heterocycloalkyl substituted with R¹², C₆-C₁₀ aryl, C₆-C₁₀ aryl substituted with R¹², 5-10 membered heteroaryl, or 5-10 membered heteroaryl substituted with R¹²; R¹² at each occurrence is independently selected from deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, and C₃-C₆ cycloalkyl.

In some preferred embodiments, R¹ represents substituted or unsubstituted C₃-C₁₀ cycloalkyl, substituted or unsubstituted 4-10 membered heterocycloalkyl, substituted or unsubstituted C₆-C₁₀ aryl, or substituted or unsubstituted 5-10 membered heteroaryl.

In some preferred embodiments, R¹ represents C₃-C₁₀ cycloalkyl, 4-10 membered heterocycloalkyl, C₆-C₁₀ aryl, or 5-10 membered heteroaryl; and the C₃-C₁₀ cycloalkyl, 4-10 membered heterocycloalkyl, C₆-C₁₀ aryl, 5-10 membered heteroaryl are further optionally substituted with at least one substituent independently selected from hydrogen, deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ heterocycloalkyl, C₆-C₁₀ aryl, 5-10 membered heteroaryl, C₁-C₆ alkyl substituted with R¹¹, C₁-C₆ alkoxy substituted with R¹¹, C₂-C₆ alkenyl substituted with R¹¹, C₂-C₆ alkynyl substituted with R¹¹, C₃-C₆ cycloalkyl substituted with R¹¹, C₃-C₆ heterocycloalkyl substituted with R¹¹, C₆-C₁₀ aryl substituted with R¹¹, and 5-10 membered heteroaryl substituted with R¹¹;
wherein R¹¹ at each occurrence is independently deuterium, halogen, cyano, nitro, C₁-C₆ alkyl, C₁-C₆ alkyl substituted with R¹², C₁-C₆ alkoxy, C₁-C₆ alkoxy substituted with R¹², C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(=NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ cycloalkyl substituted with R¹², C₃-C₆ heterocycloalkyl, C₃-C₆ heterocycloalkyl substituted with R¹², C₆-C₁₀ aryl, C₆-C₁₀ aryl substituted with R¹², 5-10 membered heteroaryl, or 5-10 membered heteroaryl substituted with R¹².
wherein R¹² at each occurrence is independently deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, or C₃-C₆ cycloalkyl.

In some embodiments, R¹ is not a ring, and R¹ represents C₁-C₆ alkyl or C₁-C₆ alkyl substituted with R¹¹, wherein R¹¹ at each occurrence is independently deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ heterocycloalkyl, C₆-C₁₀ aryl, 5-10 membered heteroaryl, C₃-C₆ heterocycloalkyl substituted with R¹², C₆-C₁₀ aryl substituted with R¹², or 5-10 membered heteroaryl substituted with R¹²; wherein each R¹² is independently deuterium, halogen, C₁-C₆ alkyl, or hydroxyl.

In some embodiments, R¹ is not a ring, and R¹ represents C₁-C₆ alkyl or C₁-C₆ alkyl substituted with R¹¹, wherein R¹¹ is phenyl, 5-10 membered heteroaryl, C₃-C₆ heterocycloalkyl, C₃-C₆ heterocycloalkyl substituted with C₁-C₆ alkyl, or C₃-C₆ cycloalkyl.

In some embodiments, R¹ is not a ring, and R¹ represents C₁-C₆ alkyl, C₁-C₆ alkyl substituted with phenyl, C₁-C₆ alkyl substituted with C₃-C₆ heterocycloalkyl, C₁-C₆ alkyl substituted with C₃-C₆ cycloalkyl, C₁-C₆ alkyl substituted with C₁-C₆ alkyl-substituted C₃-C₆ heterocycloalkyl, or C₁-C₆ alkyl substituted with 5-10 membered heteroaryl.

In some embodiments, R¹ is not a ring, and R¹ represents C₁-C₆ alkyl, or methyl or ethyl substituted with R¹¹, wherein R¹¹ at each occurrence is independently C₃-C₆ cycloalkyl, C₃-C₆ heterocycloalkyl, phenyl, 5-10 membered heteroaryl, or C₃-C₆ heterocycloalkyl substituted with C₁-C₆ alkyl.

In some embodiments, R¹ is not a ring, and R¹ represents phenyl-substituted methyl, ethyl, n-propyl, isopropyl, n-butyl, sec-butyl, isobutyl, tert-butyl, nitrogen-containing C₃-C₆ heterocycloalkyl-substituted methyl, methyl substituted with methyl-substituted nitrogen-containing C₃-C₆ heterocycloalkyl, C₃-C₆ cycloalkyl-substituted methyl, or 5-10 membered heteroaryl-substituted ethyl.

In some embodiments, R¹ is not a ring, and R¹ represents phenyl-substituted methyl, ethyl, n-propyl, isopropyl, n-butyl, sec-butyl, isobutyl, tert-butyl, 6-membered nitrogen-containing heterocycloalkyl-substituted methyl, methyl substituted with methyl-substituted 6-membered nitrogen-containing heterocycloalkyl, cyclopropyl-substituted-methyl, or 5- or 6-membered heteroaryl-substituted ethyl.

In some preferred embodiments, R¹ represents substituted or unsubstituted C₃-C₁₀ cycloalkyl, substituted or unsubstituted C₆-C₁₀ aryl, or substituted or unsubstituted 5-10 membered heteroaryl.

In some preferred embodiments, R¹ represents C₃-C₁₀ cycloalkyl, C₆-C₁₀ aryl, or 5-10 membered heteroaryl; and the C₃-C₁₀ cycloalkyl, C₆-C₁₀ aryl, 5-10 membered heteroaryl are further optionally substituted with at least one substituent independently selected from hydrogen, deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ heterocycloalkyl, C₆-C₁₀ aryl, 5-10 membered heteroaryl, C₁-C₆ alkyl substituted with R¹¹, C₁-C₆ alkoxy substituted with R¹¹, C₂-C₆ alkenyl substituted with R¹¹, C₂-C₆ alkynyl substituted with R¹¹, C₃-C₆ cycloalkyl substituted with R¹¹, C₃-C₆ heterocycloalkyl substituted with R¹¹, C₆-C₁₀ aryl substituted with R¹¹, and 5-10 membered heteroaryl substituted with R¹¹;
wherein R¹¹ at each occurrence is independently deuterium, halogen, cyano, nitro, C₁-C₆ alkyl, C₁-C₆ alkyl substituted with R¹², C₁-C₆ alkoxy, C₁-C₆ alkoxy substituted with R¹², C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(=NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ cycloalkyl substituted with R¹², C₃-C₆ heterocycloalkyl, C₃-C₆ heterocycloalkyl substituted with R¹², C₆-C₁₀ aryl, C₆-C₁₀ aryl substituted with R¹², 5-10 membered heteroaryl, or 5-10 membered heteroaryl substituted with R¹²;
wherein R¹² at each occurrence is independently deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(=NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, or C₃-C₆ cycloalkyl.

In some preferred embodiments, R¹ represents C₃-C₁₀ cycloalkyl, C₆-C₁₀ aryl, or 5-10 membered heteroaryl; and the C₃-C₁₀ cycloalkyl, C₆-C₁₀ aryl, 5-10 membered heteroaryl are further optionally is substituted with at least one substituent independently selected from hydrogen, deuterium, halogen, cyano, nitro, C₁-C₆ alkyl, C₂-C₆ alkenyl, C₂-C₆ alkynyl, C₁-C₆ alkoxy, —OR^{a}, —NR^{a}R^{b}, —SR^{a}, —S(O)₂R^{a}, —S(O)(═NR^{b})R^{a}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ heterocycloalkyl, C₆-C₁₀ aryl, 5-10 membered heteroaryl, C₁-C₆ alkyl substituted with R¹¹, C₁-C₆ alkoxy substituted with R¹¹, C₃-C₆ cycloalkyl substituted with R¹¹, C₃-C₆ heterocycloalkyl substituted with R¹¹, C₆-C₁₀ aryl substituted with R¹¹, and 5-10 membered heteroaryl substituted with R¹¹;
wherein R¹¹ at each occurrence is independently deuterium, halogen, cyano, nitro, C₁-C₆ alkyl, C₁-C₆ alkoxy, —OR^{a}, —NR^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ cycloalkyl substituted with R¹², C₃-C₆ heterocycloalkyl, C₃-C₆ heterocycloalkyl substituted with R¹²;
wherein R¹² at each occurrence is independently deuterium, halogen, C₁-C₆ alkyl, —OR^{a}, or —NR^{a}R^{b}.

In some preferred embodiments, R¹ represents C₃-C₁₀ cycloalkyl, C₆-C₁₀ aryl, or 5-10 membered heteroaryl; and the C₃-C₁₀ cycloalkyl, C₆-C₁₀ aryl, 5-10 membered heteroaryl are further optionally substituted by at least one substituent independently selected from deuterium, fluoro, chloro, bromo, iodo, cyano, nitro, amino, methylamino, dimethylamino, diethylamino, vinyl, ethynyl, hydroxy, methoxy, ethoxy, —SCH₃, —SCH₂CH₃, —S(O)₂CH₃, methyl, ethyl, n-propyl, isopropyl, n-butyl, isobutyl, sec-butyl, tert-butyl, cyclopropyl, cyclobutyl, cyclopentyl, cyclohexyl, halo-methyl, halo-ethyl, halo-n-propyl, halo-isopropyl, halo-n-butyl, halo-isobutyl, halo-sec-butyl, halo-tert-butyl, halo-cyclopropyl, phenyl-substituted methyl, 5- or 6-membered nitrogen-containing heterocycloalkyl-substituted methyl, 5- or 6-membered nitrogen-containing heterocycloalkyl-substituted ethyl, methyl substituted with C₁-C₆ alkyl-substituted 5- or 6-membered nitrogen-containing heterocycloalkyl, ethyl substituted with C₁-C₆ alkyl-substituted 5- or 6-membered nitrogen-containing heterocycloalkyl, C₃-C₆ cycloalkyl-substituted methyl, and C₃-C₆ cycloalkyl-substituted ethyl.

In some preferred embodiments, R¹ represents phenyl, naphthyl, pyridyl, pyrimidinyl, pyridazinyl, pyrazinyl, furyl, thienyl, pyrrolyl, pyrazolyl, imidazolyl, oxazolyl, thiazolyl, benzofuranyl, benzothienyl, indolyl (benzopyrrolyl), benzimidazolyl, benzoxazolyl, benzothiazolyl, quinolinyl, isoquinolinyl, acridinyl, purinyl, cyclopropyl, cyclobutyl, cyclopentyl, cyclohexyl, spiro[2.2]pentyl, spiro[2.3]hexyl, spiro[3.3]heptyl, spiro[2.4]heptyl, spiro[2.5]octyl, spiro[3.4]octyl, spiro[2.6]nonyl, spiro[3.5]nonyl, spiro[4.4]nonyl, nitrogen-containing C₅-C₁₀ spiroheterocycloalkyl, 1,2,3,4-tetrahydronaphthyl, 1,2,3,4-tetrahydroisoquinolinyl, benzo[d][1,3]dioxolyl, piperazinyl; or phenyl, naphthyl, pyridyl, pyrimidinyl, pyridazinyl, pyrazinyl, furyl, thienyl, pyrrolyl, pyrazolyl, imidazolyl, oxazolyl, thiazolyl, benzofuranyl, benzothienyl, indolyl (benzopyrrolyl), benzimidazolyl, benzoxazolyl, benzothiazolyl, quinolinyl, isoquinolinyl, acridinyl, purinyl, cyclopropyl, cyclobutyl, cyclopentyl, cyclohexyl, spiro[2.2]pentyl, spiro[2.3]hexyl, spiro[3.3]heptyl, spiro[2.4]heptyl, spiro[2.5]octyl, spiro[3.4]octyl, spiro[2.6]nonyl, spiro[3.5]nonyl, spiro[4.4]nonyl, nitrogen-containing C₅-C₁₀ spiroheterocycloalkyl, 1,2,3,4-tetrahydronaphthyl, 1,2,3,4-tetrahydroisoquinolinyl, 1,2,3,4-tetrahydroquinolinyl, benzo[d][1,3]dioxolyl, or piperazinyl, each of which is substituted with at least one substituent selected from deuterium, fluorine, chlorine, bromine, iodine, cyano, nitro, amino, methylamino, dimethylamino, diethylamino, ethynyl, hydroxy, methoxy, ethoxy, —SCH₃, —SCH₂CH₃, —S(O)₂CH₃, methyl, ethyl, n-propyl, isopropyl, n-butyl, isobutyl, sec-butyl, tert-butyl, cyclopropyl, cyclobutyl, cyclopentyl, cyclohexyl, fluoromethyl (e.g., monofluoromethyl, difluoromethyl, trifluoromethyl), fluoroethyl, fluoroisopropyl, fluoro-t-butyl, fluorocyclopropyl, trifluoromethyl-substituted cyclopropyl, trifluoromethyl-substituted isopropyl, phenyl-substituted methyl, methyl substituted with nitrogen-containing 6-membered heterocycloalkyl (e.g., piperazinyl), and methyl substituted with methyl-substituted nitrogen-containing 6-membered heterocycloalkyl (e.g., methyl substituted with 4-methylpiperazinyl).

In some preferred embodiments, R¹ represents substituted or unsubstituted C₆-C₁₀ aryl, or substituted or unsubstituted 5-10 membered heteroaryl.

In some preferred embodiments, R¹ represents C₆-C₁₀ aryl, or 5-10 membered heteroaryl; and the C₆-C₁₀ aryl, 5-10 membered heteroaryl are further optionally substituted with at least one substituent independently selected from hydrogen, deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ heterocycloalkyl, C₆-C₁₀ aryl, 5-10 membered heteroaryl, C₁-C₆ alkyl substituted with R¹¹, C₁-C₆ alkoxy substituted with R¹¹, C₂-C₆ alkenyl substituted with R¹¹, C₂-C₆ alkynyl substituted with R¹¹, C₃-C₆ cycloalkyl substituted with R¹¹, C₃-C₆ heterocycloalkyl substituted with R¹¹, C₆-C₁₀ aryl substituted with R¹¹, and 5-10 membered heteroaryl substituted with R¹¹;
wherein R¹¹ at each occurrence is independently deuterium, halogen, cyano, nitro, C₁-C₆ alkyl, C₁-C₆ alkyl substituted with R¹², C₁-C₆ alkoxy, C₁-C₆ alkoxy substituted with R¹², C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(=NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ cycloalkyl substituted with R¹², C₃-C₆ heterocycloalkyl, C₃-C₆ heterocycloalkyl substituted with R¹², C₆-C₁₀ aryl, C₆-C₁₀ aryl substituted with R¹², 5-10 membered heteroaryl, or 5-10 membered heteroaryl substituted with R¹²;
wherein R¹² at each occurrence is independently deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, or C₃-C₆ cycloalkyl.

In some preferred embodiments, the compound has a structure represented by Formula (III),

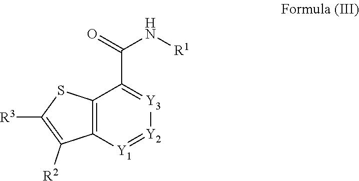

Wherein, Y₁ represents CR^{Y1} or N;
Y₂ represents CR^{Y2} or N;
Y₃ represents CR^{Y3} or N;
R¹ represents C₆-C₁₀ aryl, or 5-10 membered heteroaryl; and the C₆-C₁₀ aryl, 5-10 membered heteroaryl are further optionally substituted with at least one substituent independently selected from hydrogen, deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ heterocycloalkyl, C₆-C₁₀ aryl, 5-10 membered heteroaryl, C₁-C₆ alkyl substituted with R¹¹, C₁-C₆ alkoxy substituted with R¹¹, C₂-C₆ alkenyl substituted with R¹¹, C₂-C₆ alkynyl substituted with R¹¹, C₃-C₆ cycloalkyl substituted with R¹¹, C₃-C₆ heterocycloalkyl substituted with R¹¹, C₆-C₁₀ aryl substituted with R¹¹, and 5-10 membered heteroaryl substituted with R¹¹; wherein R¹¹ at each occurrence is independently deuterium, halogen, cyano, nitro, C₁-C₆ alkyl, C₁-C₆ alkyl substituted with R¹², C₁-C₆ alkoxy, C₁-C₆ alkoxy substituted with R¹², C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ cycloalkyl substituted with R¹², C₃-C₆ heterocycloalkyl, C₃-C₆ heterocycloalkyl substituted with R¹², C₆-C₁₀ aryl, C₆-C₁₀ aryl substituted with R¹², 5-10 membered heteroaryl, or 5-10 membered heteroaryl substituted with R¹²; R¹² at each occurrence is independently selected from deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, and C₃-C₆ cycloalkyl;
R² represents hydrogen, deuterium, halogen, hydroxy, amino, cyano, C₂-C₆ alkenyl, C₂-C₆ alkynyl, C₁-C₆ alkyl, or C₃-C₁₀ cycloalkyl; and the C₁-C₆ alkyl, C₃-C₁₀ cycloalkyl are further optionally substituted by at least one substituent independently selected from deuterium, halogen, hydroxy, C₁-C₆ alkyl, and C₃-C₁₀ cycloalkyl;
R³ represents optionally substituted or unsubstituted heteroaryl;
wherein, R^{Y1}, R^{Y2} and R^{Y3} each independently represent hydrogen, deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ heterocycloalkyl, C₆-C₁₀ aryl, 5-10 membered heteroaryl; or C₁-C₆ alkyl, C₁-C₆ alkoxy, C₂-C₆ alkenyl, C₂-C₆ alkynyl, C₃-C₆ cycloalkyl, C₃-C₆ heterocycloalkyl, C₆-C₁₀ aryl or 5-10 membered heteroaryl, each of which is substituted by at least one substituent selected from hydrogen, deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ heterocycloalkyl, C₆-C₁₀ aryl, and 5-10 membered heteroaryl;
wherein R^{a} and R^{b} each independently represent hydrogen, deuterium, halogen, cyano, hydroxy, amino, C₁-C₆ alkyl, C₁-C₆ haloalkyl, hydroxy-substituted C₁-C₆ alkyl, C₃-C₁₀ cycloalkyl, C₃-C₁₀ halocycloalkyl, or hydroxy-substituted C₃-C₁₀ cycloalkyl; or R^{a} and R^{b}, together with the atom to which they are attached, form a 3-6 membered ring, and the said ring optionally contain 1 or 2 heteroatoms selected from O, N, S, or P, and further, the said ring may optionally substituted by substituent selected from halogen, hydroxy, and C₁-C₆ alkyl; When R¹ is a substituted C₆-C₁₀ aryl or 5-10 membered heteroaryl, two adjacent substituents on the ring may, together with the atoms to which they are attached, form a 3-6 membered ring; said ring may optionally contain 0, 1, or 2 heteroatoms selected from O, N, and S.

When R³ is substituted, two adjacent substituents on the R³ ring may, together with the atoms to which they are attached, form a 3-6 membered ring; said ring may optionally contain 0, 1, or 2 heteroatoms selected from O, N, and S.

In some preferred embodiments, R¹ represents C₆-C₁₀ aryl, or 5-10 membered heteroaryl; and the C₆-C₁₀ aryl, 5-10 membered heteroaryl are further optionally substituted with at least one substituent independently selected from hydrogen, deuterium, halogen, cyano, nitro, C₁-C₆ alkyl, C₂-C₆ alkenyl, C₂-C₆ alkynyl, C₁-C₆ alkoxy, —OR^{a}, —NR^{a}R^{b}, —SR^{a}, —S(O)₂R^{a}, —S(O)(=NR^{b})R^{a}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ heterocycloalkyl, C₆-C₁₀ aryl, 5-10 membered heteroaryl, C₁-C₆ alkyl substituted with R¹¹, C₁-C₆ alkoxy substituted with R¹¹, C₃-C₆ cycloalkyl substituted with R¹¹, C₃-C₆ heterocycloalkyl substituted with R¹¹, C₆-C₁₀ aryl substituted with R¹¹, and 5-10 membered heteroaryl substituted with R¹¹;
wherein R¹¹ at each occurrence is independently deuterium, halogen, cyano, nitro, C₁-C₆ alkyl, C₁-C₆ alkoxy, —OR^{a}, —NR^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ cycloalkyl substituted with R¹², C₃-C₆ heterocycloalkyl, C₃-C₆ heterocycloalkyl substituted with R¹²;
wherein R¹² at each occurrence is independently deuterium, halogen, C₁-C₆ alkyl, —OR^{a}, or —NR^{a}R^{b}.

In some preferred embodiments, R¹ represents C₆-C₁₀ aryl, or 5-10 membered heteroaryl; and the C₆-C₁₀ aryl, 5-10 membered heteroaryl are further optionally substituted with at least one substituent independently selected from deuterium, fluoro, chloro, bromo, iodo, cyano, nitro, amino, hydroxy, methyl, ethyl, n-propyl, isopropyl, n-butyl, isobutyl, sec-butyl, tert-butyl, cyclopropyl, cyclobutyl, cyclopentyl, cyclohexyl, halo-methyl, halo-ethyl, halo-n-propyl, halo-isopropyl, halo-tert-butyl, halo-cyclopropyl, 5- or 6-membered nitrogen-containing heterocycloalkyl-substituted methyl, 5- or 6-membered nitrogen-containing heterocycloalkyl-substituted ethyl, methyl substituted with C₁-C₆ alkyl-substituted 5- or 6-membered nitrogen-containing heterocycloalkyl, and ethyl substituted with C₁-C₆ alkyl-substituted 5- or 6-membered nitrogen-containing heterocycloalkyl.

In some preferred embodiments, R¹ represents phenyl, naphthyl, pyridyl, pyrimidinyl, pyridazinyl, pyrazinyl, furyl, thienyl, pyrrolyl, pyrazolyl, imidazolyl, oxazolyl, thiazolyl, benzofuranyl, benzothienyl, indolyl (benzopyrrolyl), benzimidazolyl, benzoxazolyl, benzothiazolyl, quinolinyl, isoquinolinyl, acridinyl, or purinyl; and the phenyl, naphthyl, pyridyl, pyrimidinyl, pyridazinyl, pyrazinyl, furyl, thienyl, pyrrolyl, pyrazolyl, imidazolyl, oxazolyl, thiazolyl, benzofuranyl, benzothienyl, indolyl (benzopyrrolyl), benzimidazolyl, benzoxazolyl, benzothiazolyl, quinolinyl, isoquinolinyl, acridinyl, purinyl are independently substituted with at least one substituent selected from deuterium, fluoro, chloro, bromo, iodo, cyano, nitro, amino, hydroxy, methyl, ethyl, n-propyl, isopropyl, n-butyl, isobutyl, sec-butyl, tert-butyl, cyclopropyl, cyclobutyl, cyclopentyl, cyclohexyl, trifluoromethyl, fluoroisopropyl, fluoro-t-butyl, 1-(trifluoromethyl)cyclopropyl, 5- or 6-membered nitrogen-containing heterocycloalkyl-substituted methyl, and methyl substituted with methyl-substituted 5- or 6-membered nitrogen-containing heterocycloalkyl.

In some preferred embodiments, R¹ represents phenyl, or 5- or 6-membered heteroaryl (e.g., pyridyl); and the phenyl, 5- or 6-membered heteroaryl (e.g., pyridyl) are further optionally substituted with at least one substituent independently selected from deuterium, fluoro, chloro, bromo, iodo, cyano, nitro, amino, hydroxy, methyl, ethyl, n-propyl, isopropyl, n-butyl, isobutyl, sec-butyl, tert-butyl, cyclopropyl, cyclobutyl, cyclopentyl, cyclohexyl, trifluoromethyl, fluoroisopropyl, fluoro-t-butyl, 1-(trifluoromethyl)cyclopropyl, 5- or 6-membered nitrogen-containing heterocycloalkyl-substituted methyl, and methyl substituted with C₁-C₆ alkyl-substituted 5- or 6-membered nitrogen-containing heterocycloalkyl.

In some preferred embodiments, R¹ represents phenyl, or 5- or 6-membered heteroaryl (e.g., pyridyl); and the phenyl, 5- or 6-membered heteroaryl (e.g., pyridyl) are further optionally substituted with at least one substituent independently selected from deuterium, fluoro, chloro, methyl, tert-butyl, trifluoromethyl, cyclopropyl, fluoroisopropyl, fluoro-t-butyl, 1-(trifluoromethyl)cyclopropyl,

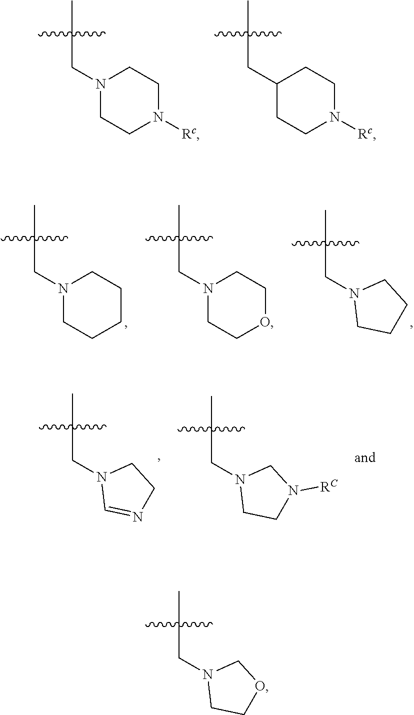

wherein R^{c} is hydrogen, C₁-C₆ alkyl, or C₃-C₁₀ cycloalkyl. Preferably, R^{c} is hydrogen, methyl, ethyl, n-propyl, isopropyl, n-butyl, isobutyl, sec-butyl, or tert-butyl. More preferably, R^{c} is methyl.

In some preferred embodiments, R¹ represents phenyl, or 5- or 6-membered heteroaryl (e.g., pyridyl); and the phenyl, 5- or 6-membered heteroaryl (e.g., pyridyl) are further optionally substituted with at least one substituent independently selected from deuterium, fluoro, chloro, methyl, tert-butyl, trifluoromethyl, cyclopropyl, and

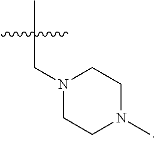 [SMILES: *CN1CCN(C)CC1]

In some preferred embodiments, R² represents hydrogen, deuterium, halogen, hydroxy, amino, cyano, C₂-C₆ alkenyl, C₂-C₆ alkynyl, C₁-C₆ alkyl, or C₃-C₁₀ cycloalkyl; and the C₁-C₆ alkyl, C₃-C₁₀ cycloalkyl are further optionally substituted with at least one substituent independently selected from deuterium, halogen, hydroxy, C₁-C₆ alkyl, and C₃-C₁₀ cycloalkyl.

In some preferred embodiments, R² represents hydrogen, deuterium, halogen, hydroxy, amino, cyano, C₂-C₆ alkenyl, C₂-C₆ alkynyl, C₁-C₆ alkyl, C₁-C₆ alkoxy, or C₃-C₁₀ cycloalkyl; and the C₁-C₆ alkyl, C₁-C₆ alkoxy, C₃-C₁₀ cycloalkyl are further optionally substituted with at least one substituent independently selected from deuterium, fluoro, chloro, bromo, iodo, hydroxy, methyl, ethyl, n-propyl, isopropyl, n-butyl, isobutyl, sec-butyl, tert-butyl, methoxy, ethoxy, cyclopropyl, cyclobutyl, cyclopentyl, and cyclohexyl.

In some preferred embodiments, R² represents hydrogen, deuterium, fluorine, chlorine, bromine, iodine, hydroxy, methyl, ethyl, n-propyl, isopropyl, n-butyl, isobutyl, sec-butyl, tert-butyl, methoxy, ethoxy, cyclopropyl, cyclobutyl, cyclopentyl, cyclohexyl, fluoromethyl, fluoroethyl, fluoro-n-propyl, fluoroisopropyl, chloromethyl, chloroethyl, chloro-n-propyl, chloroisopropyl, iodomethyl, iodoethyl, iodo-n-propyl, iodoisopropyl, fluoromethoxy, chloromethoxy, bromomethoxy, iodomethoxy, fluoroethoxy, chloroethoxy, bromoethoxy, iodoethoxy, fluoro C₃-C₆ cycloalkyl, chloro C₃-C₆ cycloalkyl, bromo C₃-C₆ cycloalkyl, or iodo C₃-C₆ cycloalkyl.

In some preferred embodiments, R² represents hydrogen, deuterium, fluorine, chlorine, bromine, iodine, hydroxy, methyl, ethyl, n-propyl, isopropyl, n-butyl, isobutyl, sec-butyl, tert-butyl, methoxy, ethoxy, cyclopropyl, cyclobutyl, cyclopentyl, cyclohexyl, trifluoromethyl, trifluoromethoxy, or fluorocyclopropyl.

In some preferred embodiments, R² represents hydrogen, deuterium, fluorine, chlorine, bromine, or iodine.

In some preferred embodiments, R³ represents substituted or unsubstituted 5-10 membered heteroaryl.

In some preferred embodiments, R³ represents substituted or unsubstituted 5- or 6-membered heteroaryl or 8-10 membered heteroaryl.

In some preferred embodiments, R³ represents substituted or unsubstituted nitrogen-containing heteroaryl.

In some preferred embodiments, R³ represents optionally substituted or unsubstituted heteroaryl containing 1-5 (preferably 1-4) heteroatoms selected from N, O, or S.

In some preferred embodiments, R³ represents optionally substituted or unsubstituted 9-10 membered heteroaryl containing 2-5 heteroatoms selected from N, O, or S.

In some preferred embodiments, R³ represents optionally substituted or unsubstituted heteroaryl containing 1-4 heteroatoms selected from N, O, or S, wherein at least one heteroatom is N.

In some preferred embodiments, R³ represents optionally substituted or unsubstituted 5- or 6-membered heteroaryl containing 1-3 heteroatoms selected from N, O, or S; or R³ represents optionally substituted or unsubstituted 8-10 membered heteroaryl containing 1-5 (preferably 1-4) heteroatoms selected from N, O, or S.

In some preferred embodiments, R³ represents optionally substituted or unsubstituted 5- or 6-membered heteroaryl containing 1-3 nitrogen atoms.

In some preferred embodiments, R³ represents optionally substituted or unsubstituted 8-10 membered heteroaryl containing 1-4 nitrogen atoms.

In some preferred embodiments, R³ represents optionally substituted or unsubstituted 9-membered heteroaryl containing 2-4 nitrogen atoms.

In some preferred embodiments, R³ represents optionally substituted or unsubstituted 8-10 membered heteroaryl containing at least one nitrogen atom and optionally containing one sulfur or oxygen atom.

In some preferred embodiments, R³ represents optionally substituted or unsubstituted 9- or 10-membered heteroaryl containing 1-4 nitrogen atoms and optionally containing one sulfur or oxygen atom.

In some preferred embodiments, R³ represents optionally substituted or unsubstituted 8-10 membered heteroaryl containing 1-3 nitrogen atoms and one sulfur or oxygen atom.

In some preferred embodiments, R³ represents optionally substituted or unsubstituted 9-membered heteroaryl containing 1-3 nitrogen atoms and one sulfur or oxygen atom; preferably, the sulfur or oxygen atom is located on the 5-membered heteroaromatic ring.

In some preferred embodiments, R³ represents 5-10 membered heteroaryl or 5-10 membered heteroaryl substituted with at least one substituent selected from hydrogen, deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ heterocycloalkyl, C₆-C₁₀ aryl, 5-10 membered heteroaryl, C₁-C₆ alkyl substituted with R³¹, C₁-C₆ alkoxy substituted with R³¹, C₂-C₆ alkenyl substituted with R³¹, C₂-C₆ alkynyl substituted with R³¹, C₃-C₆ cycloalkyl substituted with R³¹, C₃-C₆ heterocycloalkyl substituted with R³¹, C₆-C₁₀ aryl substituted with R³¹, and 5-10 membered heteroaryl substituted with R³¹;
wherein, R³¹ at each occurrence is independently deuterium, halogen, cyano, nitro, C₁-C₆ alkyl, C₁-C₆ alkyl substituted with R³², C₁-C₆ alkoxy, C₁-C₆ alkoxy substituted with R³², C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(=NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ cycloalkyl substituted with R³², C₃-C₆ heterocycloalkyl, C₃-C₆ heterocycloalkyl substituted with R³², C₆-C₁₀ aryl, C₆-C₁₀ aryl substituted with R³², 5-10 membered heteroaryl, or 5-10 membered heteroaryl substituted with R³²;
R³² at each occurrence is independently selected from deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, and —Si(R^{a})₃.

In some preferred embodiments, R³ represents 5-10 membered heteroaryl or 5-10 membered heteroaryl substituted with at least one substituent selected from hydrogen, deuterium, halogen, nitro, amino, hydroxy, cyano, C₁-C₆ alkyl, C₁-C₆ alkoxy, C₃-C₆ cycloalkyl, C₃-C₆ heterocycloalkyl, C₆-C₁₀ aryl, 5-10 membered heteroaryl, C₁-C₆ haloalkyl, C₁-C₆ haloalkoxy, C₃-C₆ halocycloalkyl, hydroxy-substituted C₁-C₆ alkyl, C₃-C₆ heterocycloalkyl-substituted C₁-C₆ alkyl, —NR^{a}R^{b}-substituted C₁-C₆ alkyl, —NR^{a}R-substituted C₁-C₆ alkoxy, C₃-C₆ heterocycloalkyl-substituted C₁-C₆ alkoxy, C₁-C₆ alkyl-substituted C₃-C₆ heterocycloalkyl, C₃-C₆ heterocycloalkyl substituted with hydroxy-substituted C₁-C₆ alkyl, C₁-C₆ alkyl substituted with —Si(R^{a})₃ (e.g., silyl)-substituted C₁-C₆ alkoxy, C₁-C₆ alkyl-substituted 5-10 membered heteroaryl, and 5-10 membered heteroaryl substituted with hydroxy-substituted C₁-C₆ alkyl.

In some preferred embodiments, R³ represents 5-10 membered heteroaryl or 5-10 membered heteroaryl substituted with at least one substituent selected from hydrogen, deuterium, fluoro, chloro, bromo, iodo, nitro, amino, hydroxy, cyano, methyl, ethyl, n-propyl, isopropyl, n-butyl, isobutyl, sec-butyl, tert-butyl, methoxy, ethoxy, cyclopropyl, cyclobutyl, cyclopentyl, cyclohexyl, trifluoromethyl, trifluoromethoxy, fluorocyclopropyl, hydroxyethyl, methoxyethyl, dimethylaminoethyl, dimethylaminoethoxy, methylsulfonylethyl, hydroxypropyl, methoxypropyl, dimethylaminopropyl, dimethylaminopropoxy, methylsulfonylpropyl, azetidinyl-substituted ethyl, azetidinyl-substituted propyl, azetidinyl-substituted ethoxy, azetidinyl-substituted propoxy, pyrrolidinyl-substituted ethyl, pyrrolidinyl-substituted propyl, pyrrolidinyl-substituted ethoxy, pyrrolidinyl-substituted propoxy, piperidinyl-substituted ethyl, piperidinyl-substituted propyl, piperidinyl-substituted ethoxy, piperidinyl-substituted propoxy, 4-methylpiperidinyl-substituted methyl

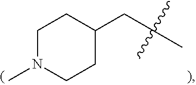 [SMILES: *CC1CCN(C)CC1]

4-methylpiperidinyl-substituted methoxy

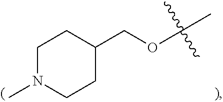 [SMILES: *OCC1CCN(C)CC1]

### N-methylpiperazinyl

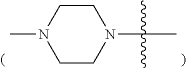 [SMILES: *N1CCN(C)CC1]

substituted ethoxy N-methylpiperazinyl

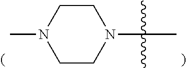 [SMILES: *N1CCN(C)CC1]

substituted ethyl, morpholinyl

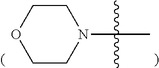 [SMILES: *N1CCOCC1]

substituted ethoxy, morpholinyl

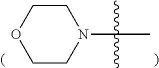 [SMILES: *N1CCOCC1]

substituted ethyl, N-methylpiperazinyl

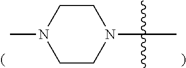 [SMILES: *N1CCN(C)CC1]

substituted propoxy, N-methylpiperazinyl

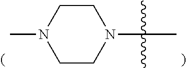 [SMILES: *N1CCN(C)CC1]

substituted propyl, morpholinyl

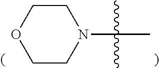 [SMILES: *N1CCOCC1]

substituted propoxy, morpholinyl

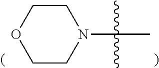 [SMILES: *N1CCOCC1]

substituted propyl, 3-methylpiperidinyl

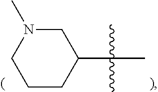 [SMILES: *C1CCCN(C)C1]

4-methylpiperidinyl

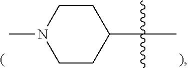 [SMILES: *C1CCN(C)CC1]

3-methylpyrrolidinyl

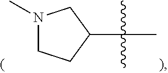 [SMILES: *C1CCN(C)C1]

3-methylazetidinyl

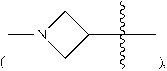 [SMILES: *C1CN(C)C1]

oxetanyl, oxolanyl, oxanyl, hydroxymethyl (—CH₂OH)-oxolanyl, methyl substituted with silomethyl-substituted ethyl, 3-methyl-3-(4-methyl-1-piperazinyl)-1-butyn-1-yl

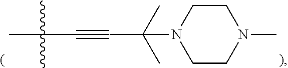 [SMILES: *C#CC(C)(C)N1CCN(C)CC1]

3-methyl-3-amino-1-butyn-1-yl

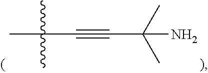 [SMILES: *C#CC(C)(C)N]

methyl-substituted pyrazolyl, hydroxyethyl-substituted pyrazolyl, imidazolyl, and pyridinyl.

In some preferred embodiments, R³ represents substituted or unsubstituted

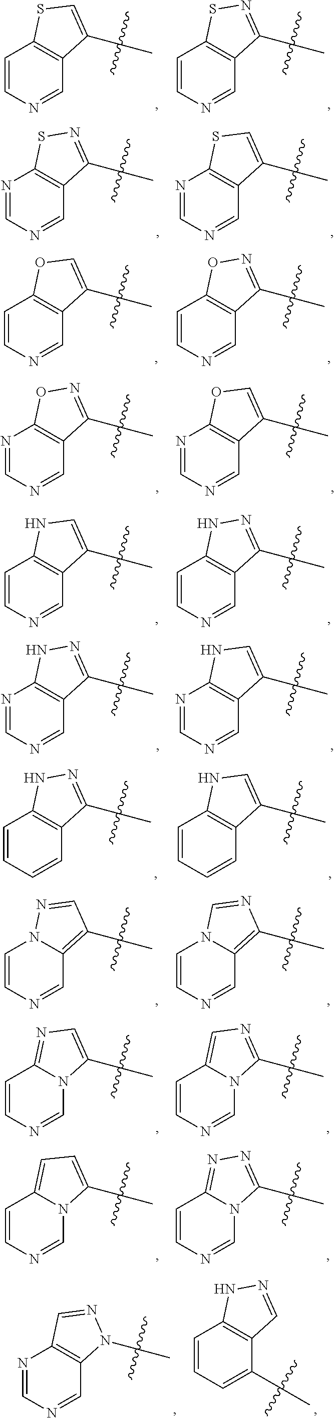

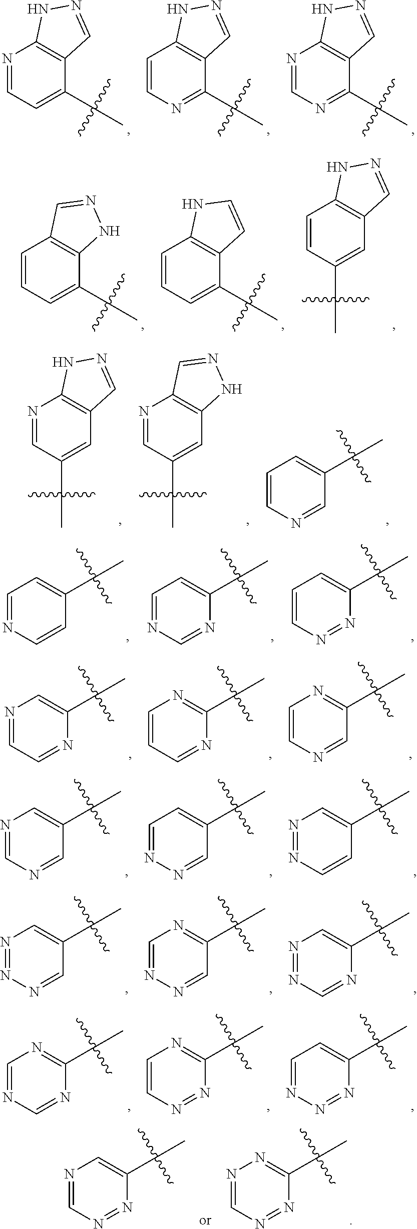

In some preferred embodiments, R³ represents

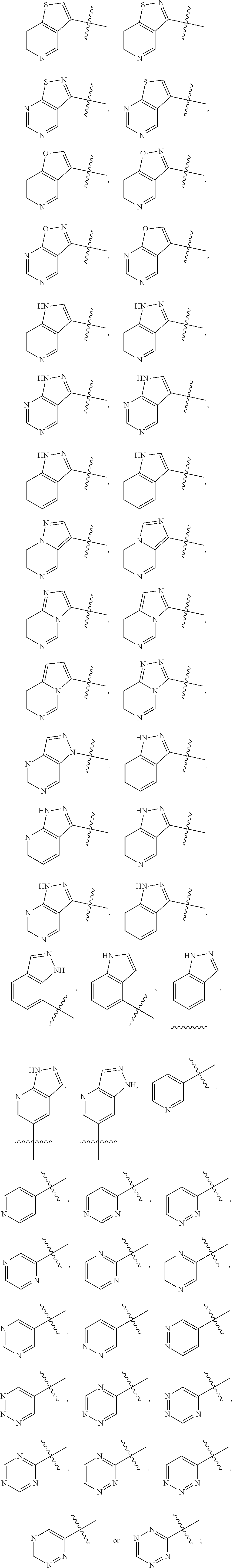

and the

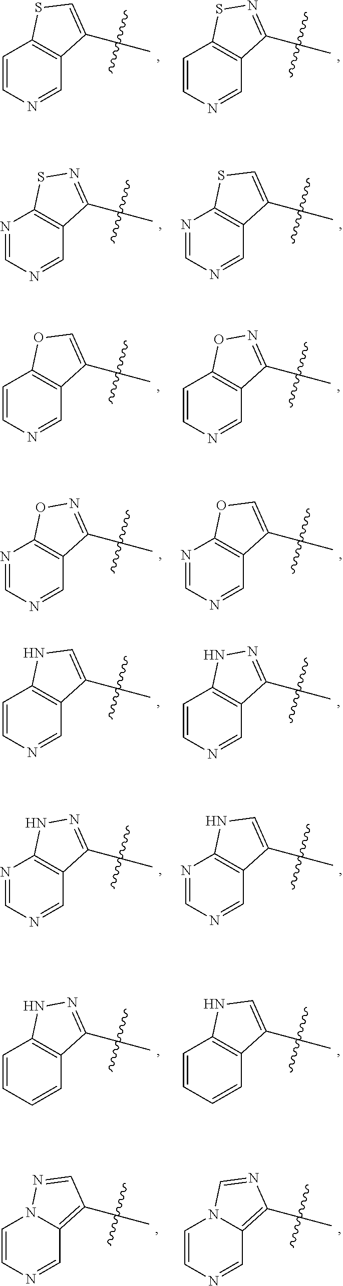

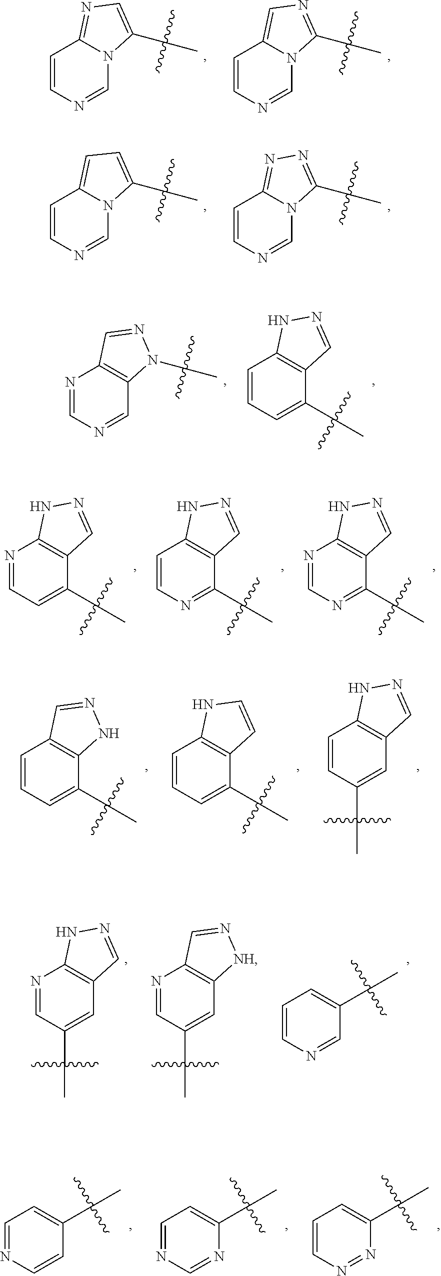

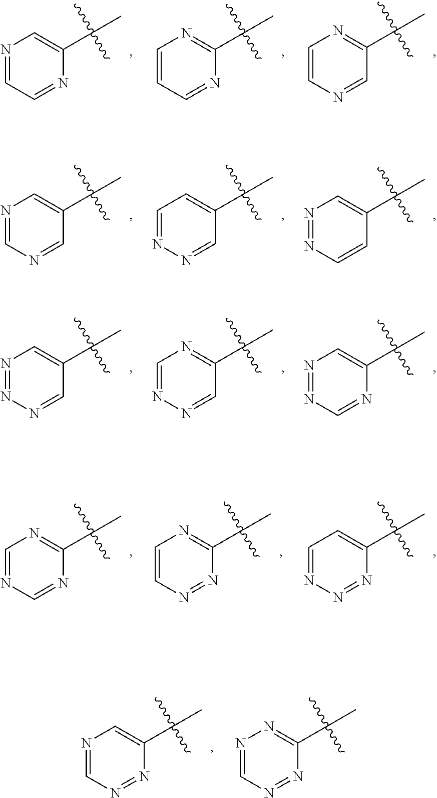

are further optionally substituted with at least one substituent independently selected from hydrogen, deuterium, fluoro, chloro, bromo, iodo, amino, hydroxy, methyl, ethyl, n-propyl, isopropyl, methoxy, ethoxy, cyclopropyl, cyclobutyl, cyclopentyl, cyclohexyl, trifluoromethyl, trifluoromethoxy, fluorocyclopropyl, cyano, hydroxyethyl, methoxyethyl, dimethylaminoethyl, dimethylaminoethoxy, methylsulfonylethyl, hydroxypropyl, methoxypropyl, dimethylaminopropyl, dimethylaminopropoxy, methylsulfonylpropyl, azetidinyl-substituted ethyl, azetidinyl-substituted propyl, azetidinyl-substituted ethoxy, azetidinyl-substituted propoxy, pyrrolidinyl-substituted ethyl, pyrrolidinyl-substituted propyl, pyrrolidinyl-substituted ethoxy, pyrrolidinyl-substituted propoxy, piperidinyl-substituted ethyl, piperidinyl-substituted propyl, piperidinyl-substituted ethoxy, piperidinyl-substituted propoxy, 4-methylpiperidinyl-substituted methyl

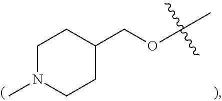 [SMILES: *OCC1CCN(C)CC1]

4-methylpiperidinyl-substituted methoxy

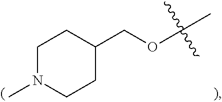 [SMILES: *OCC1CCN(C)CC1]

### N-methylpiperazinyl

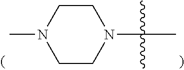 [SMILES: *N1CCN(C)CC1]

substituted ethoxy, N-methylpiperazinyl

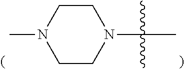 [SMILES: *N1CCN(C)CC1]

substituted ethyl, morpholinyl

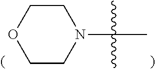 [SMILES: *N1CCOCC1]

substituted ethoxy, morpholinyl

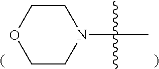 [SMILES: *N1CCOCC1]

substituted ethyl, N-methylpiperazinyl

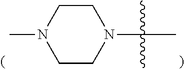 [SMILES: *N1CCN(C)CC1]

substituted propoxy, N-methylpiperazinyl

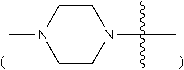 [SMILES: *N1CCN(C)CC1]

substituted propyl, morpholinyl

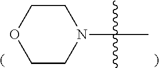 [SMILES: *N1CCOCC1]

substituted propoxy, morpholinyl

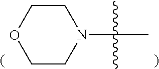 [SMILES: *N1CCOCC1]

substituted propyl, 3-methylpiperidinyl

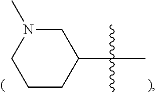 [SMILES: *C1CCCN(C)C1]

4-methylpiperidinyl

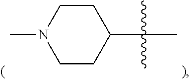 [SMILES: *C1CCN(C)CC1]

3-methylpyrrolidinyl

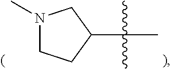 [SMILES: *C1CCN(C)C1]

methylazetidinyl

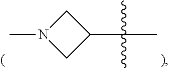 [SMILES: *C1CN(C)C1]

oxetanyl, oxolanyl, oxanyl, hydroxymethyl (—CH₂OH)-oxolanyl, silomethyl-substituted ethyl-substituted methyl, 3-methyl-3-(4-methyl-1-piperazinyl)-1-butyn-1-yl

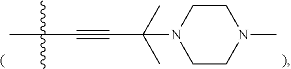 [SMILES: *C#CC(C)(C)N1CCN(C)CC1]

3-methyl-3-amino-1-butyn-1-yl

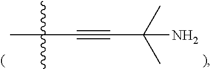 [SMILES: *C#CC(C)(C)N]

methyl-substituted pyrazolyl, hydroxyethyl-substituted pyrazolyl, imidazolyl, pyridinyl.

In some more preferred embodiments, R³ represents substituted or unsubstituted

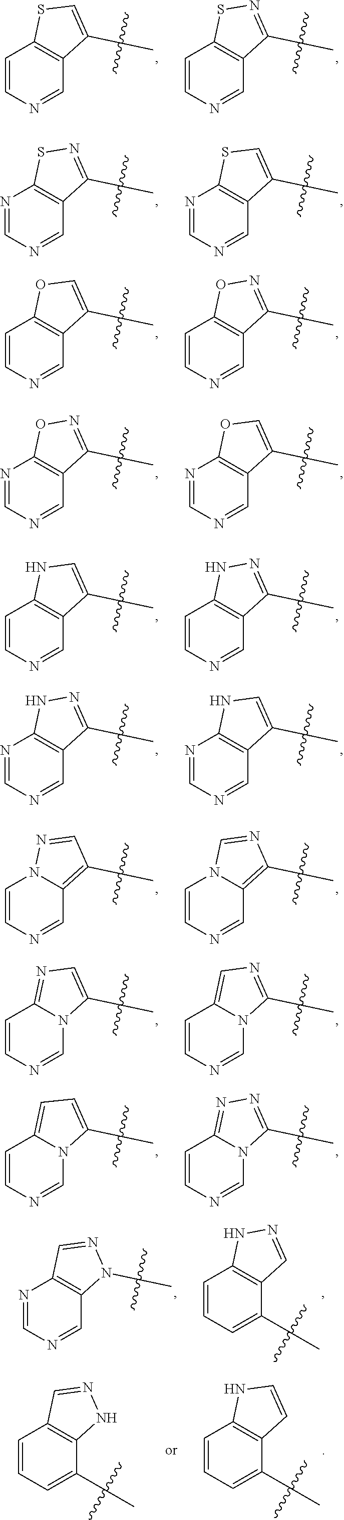

In some more preferred embodiments, R³ represents

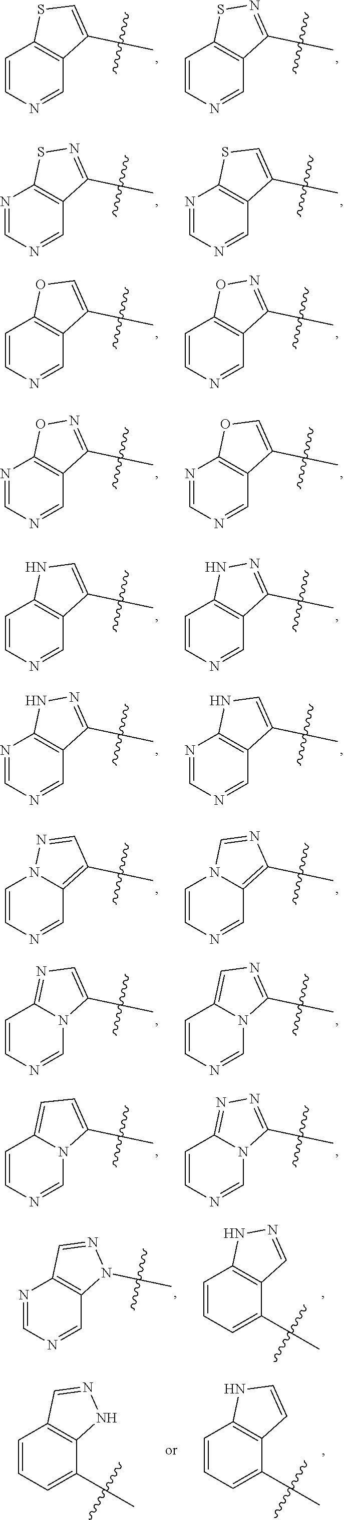

each of which is substituted with at least one substituent selected from hydrogen, deuterium, fluoro, chloro, bromo, iodo, amino, hydroxy, methyl, ethyl, n-propyl, isopropyl, methoxy, ethoxy, cyclopropyl, cyclobutyl, cyclopentyl, cyclohexyl, trifluoromethyl, trifluoromethoxy, fluorocyclopropyl, cyano, hydroxyethyl, methoxyethyl, dimethylaminoethyl, dimethylaminoethoxy, methylsulfonylethyl, hydroxypropyl, methoxypropyl, dimethylaminopropyl, dimethylaminopropoxy, methylsulfonylpropyl, azetidinyl-substituted ethyl, azetidinyl-substituted propyl, azetidinyl-substituted ethoxy, azetidinyl-substituted propoxy, pyrrolidinyl-substituted ethyl, pyrrolidinyl-substituted propyl, pyrrolidinyl-substituted ethoxy, pyrrolidinyl-substituted propoxy, piperidinyl-substituted ethyl, piperidinyl-substituted propyl, piperidinyl-substituted ethoxy, and piperidinyl-substituted propoxy, 4-methylpiperidinyl-substituted methyl

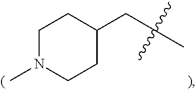 [SMILES: *CC1CCN(C)CC1]

4-methylpiperidinyl-substituted methoxy

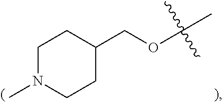 [SMILES: *OCC1CCN(C)CC1]

### N-methylpiperazinyl

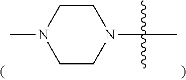 [SMILES: *N1CCN(C)CC1]

substituted ethoxy, N-methylpiperazinyl

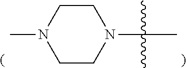 [SMILES: *N1CCN(C)CC1]

substituted ethyl, morpholinyl

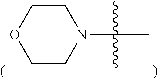 [SMILES: *N1CCOCC1]

substituted ethoxy, morpholinyl

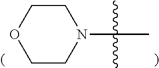 [SMILES: *N1CCOCC1]

substituted ethyl, N-methylpiperazinyl

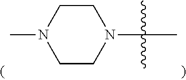 [SMILES: *N1CCN(C)CC1]

substituted propoxy, N-methylpiperazinyl

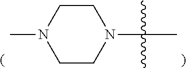 [SMILES: *N1CCN(C)CC1]

substituted propyl, morpholinyl

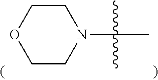 [SMILES: *N1CCOCC1]

substituted propoxy, morpholinyl

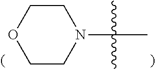 [SMILES: *N1CCOCC1]

substituted propyl, 3-methylpiperidinyl

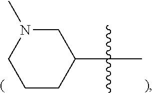 [SMILES: *C1CCCN(C)C1]

4-methylpiperidinyl

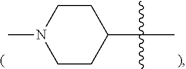 [SMILES: *C1CCN(C)CC1]

3-methylpyrrolidinyl

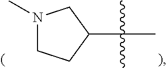 [SMILES: *C1CCN(C)C1]

3-methylazetidinyl

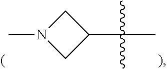 [SMILES: *C1CN(C)C1]

oxetanyl, oxolanyl, oxanyl, hydroxymethyl (—CH₂OH)-oxolanyl, methyl substituted with silomethyl-substituted ethyl, 3-methyl-3-(4-methyl-1-piperazinyl)-1-butyn-1-yl

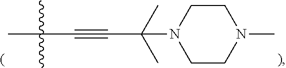 [SMILES: *C#CC(C)(C)N1CCN(C)CC1]

3-methyl-3-amino-1-butyn-1-yl

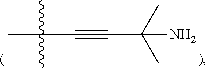 [SMILES: *C#CC(C)(C)N]

methyl substituted pyrazolyl, hydroxyethyl-substituted pyrazolyl, imidazolyl, and pyridinyl.

In some more preferred embodiments, R³ represents substituted or unsubstituted

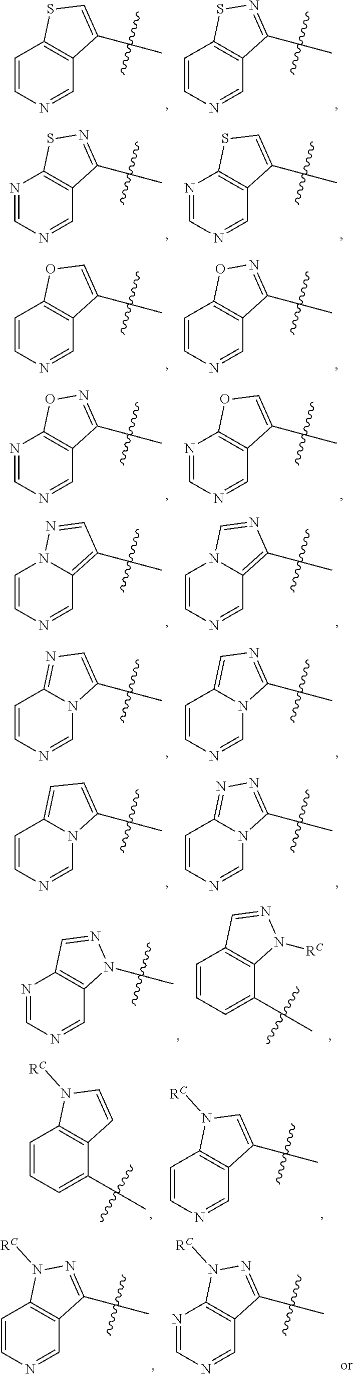

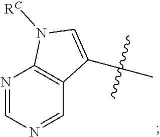

wherein R^{c} represents hydrogen, C₁-C₆ alkyl, C₁-C₆ alkoxy, C₃-C₁₀ cycloalkyl, hydroxy-substituted C₁-C₆ alkyl, C₃-C₆ heterocycloalkyl-substituted C₁-C₆ alkyl, C₃-C₆ heterocycloalkyl-substituted C₁-C₆ alkoxy, C₁-C₆ alkyl-substituted C₃-C₆ heterocycloalkyl, —NR^{a}R-substituted C₁-C₆ alkyl, or —NR^{a}R-substituted C₁-C₆ alkoxy. Preferably, R^{c} represents hydrogen, methyl, ethyl, n-propyl, isopropyl, n-butyl, isobutyl, sec-butyl, tert-butyl, hydroxyethyl, methoxyethyl, dimethylaminoethyl, methylsulfonylethyl, hydroxypropyl, methoxypropyl, dimethylaminopropyl, methylsulfonylpropyl, azetidinyl-substituted ethyl, azetidinyl-substituted propyl, pyrrolidinyl-substituted ethyl, pyrrolidinyl-substituted propyl, piperidinyl-substituted ethyl, piperidinyl-substituted propyl, 4-methylpiperidinyl-substituted methyl

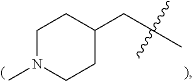 [SMILES: *CC1CCN(C)CC1]

### N-methylpiperazinyl

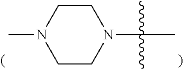 [SMILES: *N1CCN(C)CC1]

substituted ethyl, morpholinyl

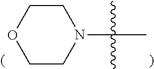 [SMILES: *N1CCOCC1]

substituted ethyl, N-methylpiperzinyl

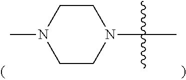 [SMILES: *N1CCN(C)CC1]

substituted propyl, morpholinyl

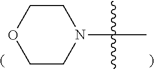 [SMILES: *N1CCOCC1]

substituted propyl, 3-methylpiperidinyl

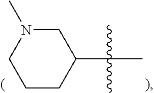 [SMILES: *C1CCCN(C)C1]

4-methylpiperidinyl

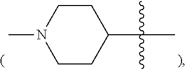 [SMILES: *C1CCN(C)CC1]

3-methylpyrrolidinyl

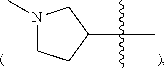 [SMILES: *C1CCN(C)C1]

3-methylazetidinyl

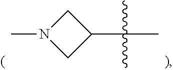 [SMILES: *C1CN(C)C1]

oxetanyl, tetrahydrofuranyl, tetrahydropyranyl; and more preferably, R^{c} represents methyl.

In some more preferred embodiments, R³ represents

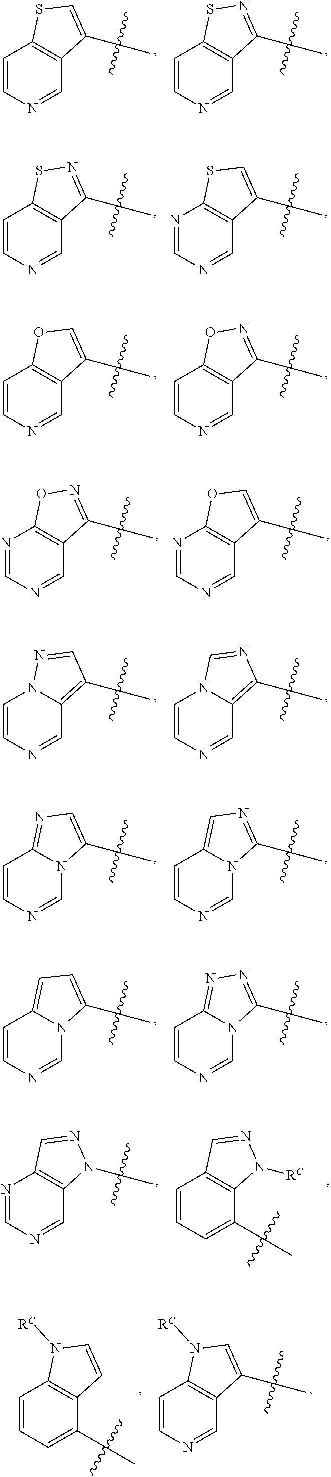

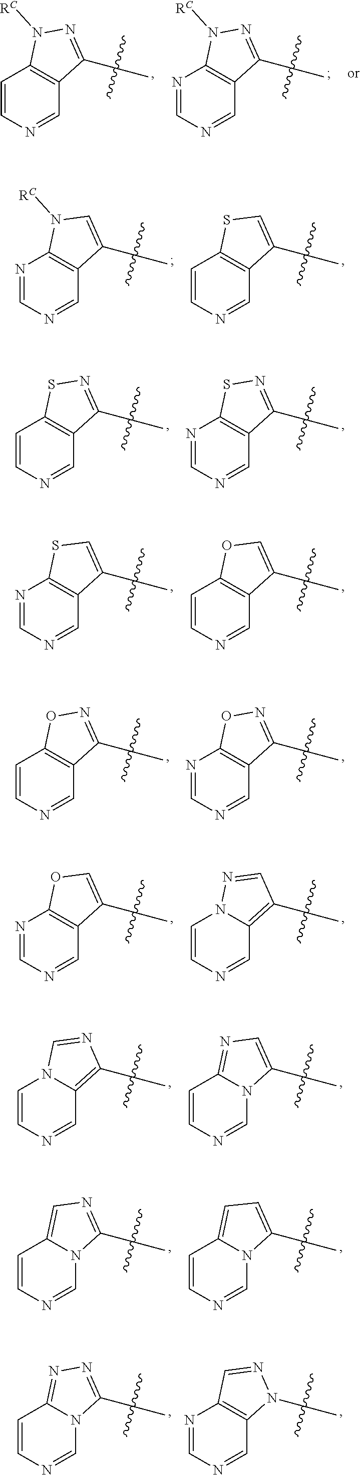

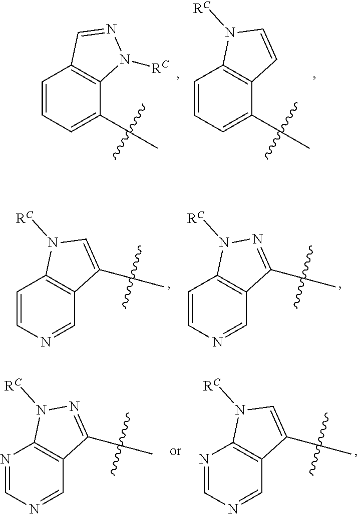

each of which is substituted with at least one substituent selected from hydrogen, deuterium, fluoro, chloro, bromo, iodo, amino, hydroxy, methyl, ethyl, n-propyl, isopropyl, n-butyl, isobutyl, sec-butyl, tert-butyl, methoxy, ethoxy, cyclopropyl, cyclobutyl, cyclopentyl, cyclohexyl, trifluoromethyl, trifluoromethoxy, fluorocyclopropyl, cyano, hydroxyethyl, methoxyethyl, dimethylaminoethyl, dimethylaminoethoxy, methylsulfonylethyl, hydroxypropyl, methoxypropyl, dimethylaminopropyl, dimethylaminopropoxy, methylsulfonylpropyl, azetidinyl-substituted ethyl, azetidinyl-substituted propyl, azetidinyl-substituted ethoxy, azetidinyl-substituted propoxy, pyrrolidinyl-substituted ethyl, pyrrolidinyl-substituted propyl, pyrrolidinyl-substituted ethoxy, pyrrolidinyl-substituted propoxy, piperidinyl-substituted ethyl, piperidinyl-substituted propyl, piperidinyl-substituted ethoxy, piperidinyl-substituted propoxy, 4-methylpiperidinyl-substituted methyl

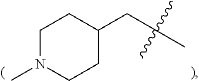 [SMILES: *CC1CCN(C)CC1]

4-methylpiperidinyl-substituted methoxy

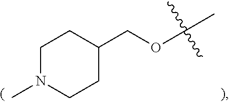 [SMILES: *OCC1CCN(C)CC1]

### N-methylpiperazinyl

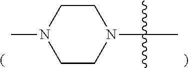 [SMILES: *N1CCN(C)CC1]

substituted ethoxy, N-methylpiperazinyl

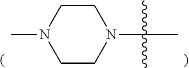 [SMILES: *N1CCN(C)CC1]

substituted ethyl, morpholinyl

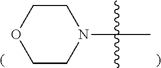 [SMILES: *N1CCOCC1]

substituted ethoxy, morpholinyl

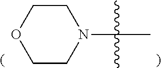 [SMILES: *N1CCOCC1]

substituted ethyl, N-methylpiperazinyl

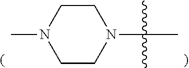 [SMILES: *N1CCN(C)CC1]

substituted propoxy, N-methylpiperazinyl

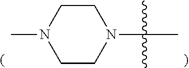 [SMILES: *N1CCN(C)CC1]

substituted propyl, morpholinyl

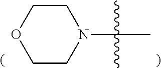 [SMILES: *N1CCOCC1]

substituted propoxy, morpholinyl

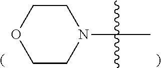 [SMILES: *N1CCOCC1]

substituted propyl, 3-methylpiperidinyl

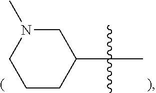 [SMILES: *C1CCCN(C)C1]

4-methylpiperidinyl

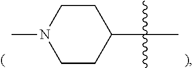 [SMILES: *C1CCN(C)CC1]

3-methylpyrrolidinyl

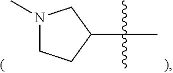 [SMILES: *C1CCN(C)C1]

3-methylazetidinyl

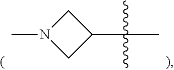 [SMILES: *C1CN(C)C1]

oxetanyl, oxolanyl, oxanyl, hydroxymethyl (—CH₂OH)-oxolanyl, methyl substituted with silomethyl-substituted ethyl, 3-methyl-3-(4-methyl-1-piperazinyl)-1-butyn-1-yl

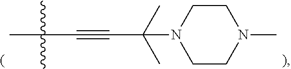 [SMILES: *C#CC(C)(C)N1CCN(C)CC1]

3-methyl-3-amino-1-butyn-1-yl

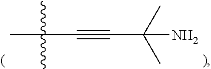 [SMILES: *C#CC(C)(C)N]

methyl-substituted pyrazolyl, hydroxyethyl-substituted pyrazolyl, imidazolyl, and pyridinyl; wherein R^{c} represents hydrogen, C₁-C₆ alkyl, C₁-C₆ alkoxy, C₃-C₁₀ cycloalkyl, hydroxy-substituted C₁-C₆ alkyl, C₃-C₆ heterocycloalkyl-substituted C₁-C₆ alkyl, C₃-C₆ heterocycloalkyl-substituted C₁-C₆ alkoxy, C₁-C₆ alkyl-substituted C₃-C₆ heterocycloalkyl, —NR^{a}R^{b}-substituted C₁-C₆ alkyl, or —NR^{a}R^{b}-substituted C₁-C₆ alkoxy. Preferably, R^{c} represents hydrogen, methyl, ethyl, n-propyl, isopropyl, n-butyl, isobutyl, sec-butyl, tert-butyl, hydroxyethyl, methoxyethyl, dimethylaminoethyl, methylsulfonylethyl, hydroxypropyl, methoxypropyl, dimethylaminopropyl, methylsulfonylpropyl, azetidinyl-substituted ethyl, azetidinyl-substituted propyl, pyrrolidinyl-substituted ethyl, pyrrolidinyl-substituted propyl, piperidinyl-substituted ethyl, piperidinyl-substituted propyl, 4-methylpiperidinyl-substituted methyl

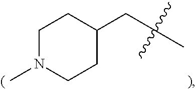 [SMILES: *CC1CCN(C)CC1]

### N-methylpiperazinyl

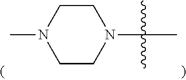 [SMILES: *N1CCN(C)CC1]

substituted ethyl, morpholinyl

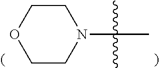 [SMILES: *N1CCOCC1]

substituted ethyl, N-methylpiperazinyl

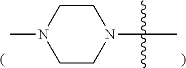 [SMILES: *N1CCN(C)CC1]

substituted propyl, morpholinyl

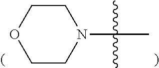 [SMILES: *N1CCOCC1]

substituted propyl, 3-methylpiperidinyl

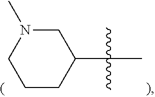 [SMILES: *C1CCCN(C)C1]

4-methylpyrrolidinyl

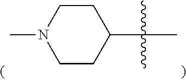 [SMILES: *C1CCN(C)CC1]

3-methylpyrrolidinyl

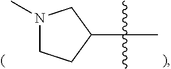 [SMILES: *C1CCN(C)C1]

3-methylazetidinyl

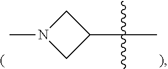 [SMILES: *C1CN(C)C1]

oxetanyl, tetrahydrofuranyl, tetrahydropyranyl; and more preferably, R^{c} represents methyl.

In some more preferred embodiments, R³ represents substituted or unsubstituted

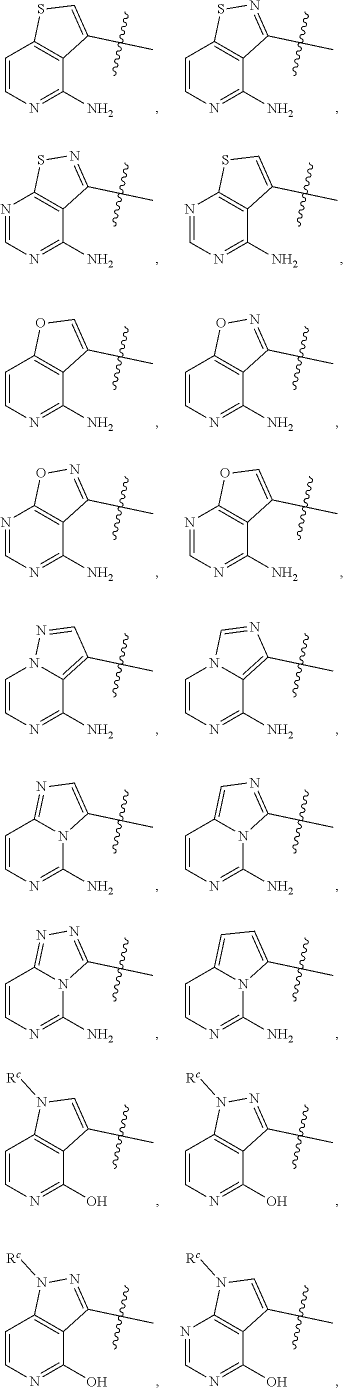

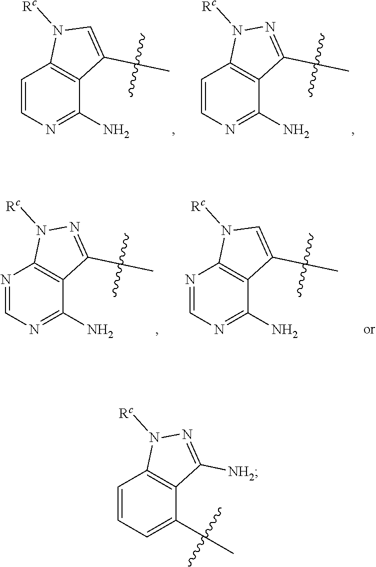

wherein R^{c} represents hydrogen, C₁-C₆ alkyl, C₁-C₆ alkoxy, C₃-C₁₀ cycloalkyl, hydroxy-substituted C₁-C₆ alkyl, C₃-C₆ heterocycloalkyl-substituted C₁-C₆ alkyl, C₃-C₆ heterocycloalkyl-substituted C₁-C₆ alkoxy, C₁-C₆ alkyl-substituted C₃-C₆ heterocycloalkyl, —NR^{a}R-substituted C₁-C₆ alkyl, or —NR^{a}R^{b}-substituted C₁-C₆ alkoxy. Preferably, R^{c} represents hydrogen, methyl, ethyl, n-propyl, isopropyl, n-butyl, isobutyl, sec-butyl, tert-butyl, hydroxyethyl, methoxyethyl, dimethylaminoethyl, methylsulfonylethyl, hydroxypropyl, methoxypropyl, dimethylaminopropyl, methylsulfonylpropyl, azetidinyl-substituted ethyl, azetidinyl-substituted propyl, pyrrolidinyl-substituted ethyl, pyrrolidinyl-substituted propyl, piperidinyl-substituted ethyl, piperidinyl-substituted propyl, 4-methylpiperidinyl-substituted methyl

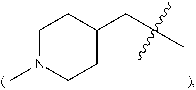 [SMILES: *CC1CCN(C)CC1]

### N-methylpiperazinyl

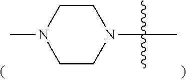 [SMILES: *N1CCN(C)CC1]

substituted ethyl, morpholinyl

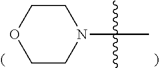 [SMILES: *N1CCOCC1]

substituted ethyl, N-methylpiperazinyl

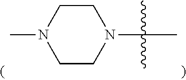 [SMILES: *N1CCN(C)CC1]

substituted propyl, morpholinyl

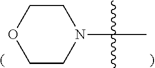 [SMILES: *N1CCOCC1]

substituted propyl, 3-methylpiperidinyl

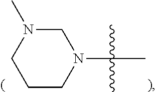 [SMILES: *N1CCCN(C)C1]

4-methylpiperidinyl

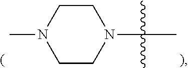 [SMILES: *N1CCN(C)CC1]

3-methylpyrrolidinyl

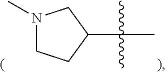 [SMILES: *C1CCN(C)C1]

3-methylazetidinyl

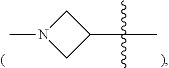 [SMILES: *C1CN(C)C1]

oxetanyl, tetrahydrofuranyl, tetrahydropyranyl; and more preferably, R^{c} represents methyl.

In some more preferred embodiments, R³ represents

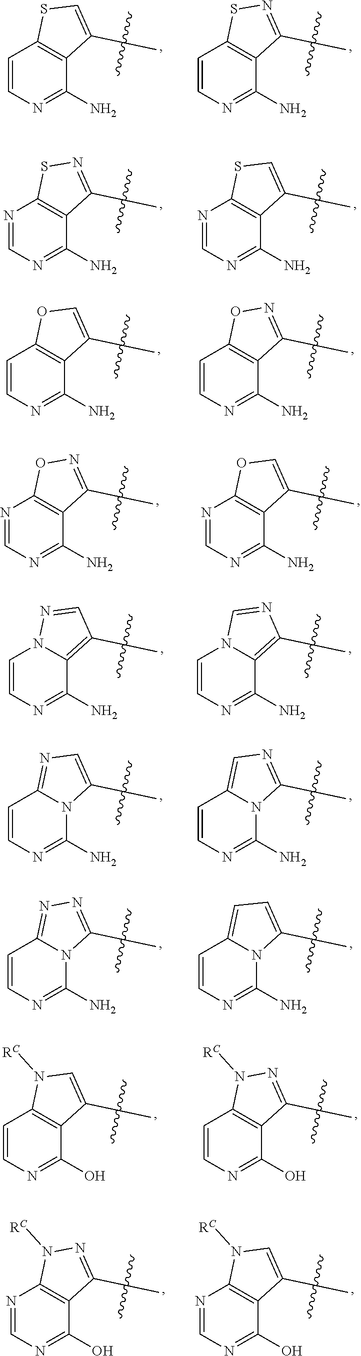

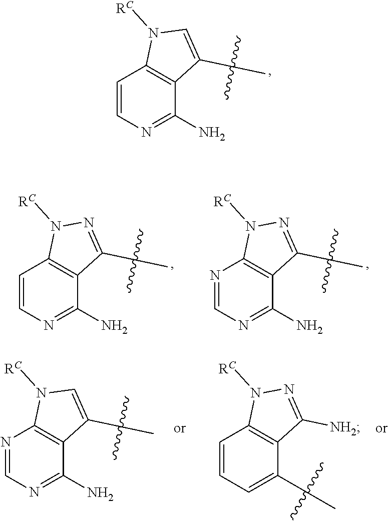

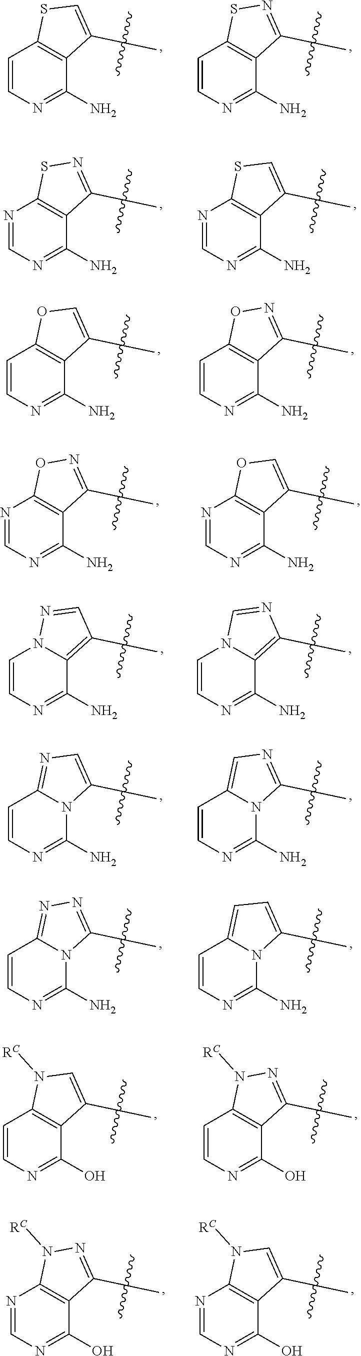

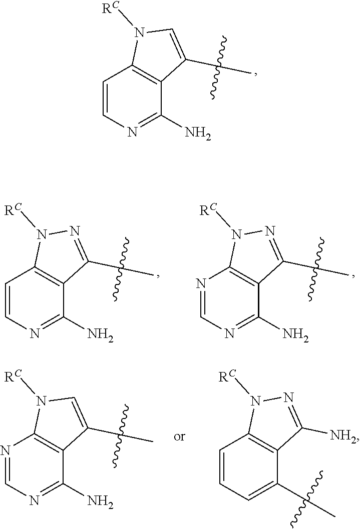

each of which is substituted with at least one substituent selected from hydrogen, deuterium, fluoro, chloro, bromo, iodo, methyl, ethyl, n-propyl, isopropyl, n-butyl, isobutyl, sec-butyl, tert-butyl, methoxy, ethoxy, cyclopropyl, cyclobutyl, cyclopentyl, cyclohexyl, trifluoromethyl, trifluoromethoxy, fluorocyclopropyl, cyano, hydroxyethyl, methoxyethyl, dimethylaminoethyl, dimethylaminoethoxy, methylsulfonylethyl, hydroxypropyl, methoxypropyl, dimethylaminopropyl, dimethylaminopropoxy, methylsulfonylpropyl, azetidinyl-substituted ethyl, azetidinyl-substituted propyl, azetidinyl-substituted ethoxy, azetidinyl-substituted propoxy, pyrrolidinyl-substituted ethyl, pyrrolidinyl-substituted propyl, pyrrolidinyl-substituted ethoxy, pyrrolidinyl-substituted propoxy, piperidinyl-substituted ethyl, piperidinyl-substituted propyl, piperidinyl-substituted ethoxy, piperidinyl-substituted propoxy, 4-methylpiperidinyl-substituted methyl

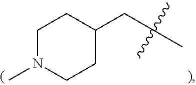 [SMILES: *CC1CCN(C)CC1]

4-methylpiperidinyl-substituted methoxy

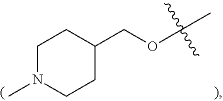 [SMILES: *OCC1CCN(C)CC1]

### N-methylpiperazinyl

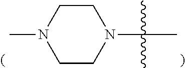 [SMILES: *N1CCN(C)CC1]

substituted ethoxy, N-methylpiperazinyl

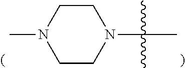 [SMILES: *N1CCN(C)CC1]

substituted ethyl, morpholinyl

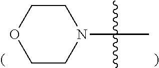 [SMILES: *N1CCOCC1]

substituted ethoxy, morpholinyl

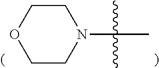 [SMILES: *N1CCOCC1]

substituted ethyl, N-methylpiperazinyl

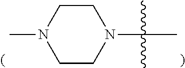 [SMILES: *N1CCN(C)CC1]

substituted propoxy, N-methylpiperazinyl

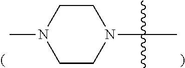 [SMILES: *N1CCN(C)CC1]

substituted propyl, morpholinyl

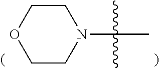 [SMILES: *N1CCOCC1]

substituted propoxy, morpholinyl

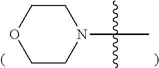 [SMILES: *N1CCOCC1]

substituted propyl, 3-methylpiperidinyl

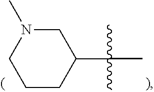 [SMILES: *C1CCCN(C)C1]

4-methylpiperidinyl

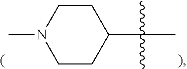 [SMILES: *C1CCN(C)CC1]

3-methylpyrrolidinyl

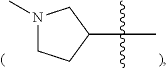 [SMILES: *C1CCN(C)C1]

3-methylazetidinyl

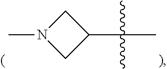 [SMILES: *C1CN(C)C1]

oxetanyl, oxolanyl, oxanyl, hydroxyniethyl (—CH₂OH)-oxolanyl, methyl substituted with silomethyl-substituted ethyl, 3-methyl-3-(4-methyl-1-piperazinyl)-1-butyn-1-yl

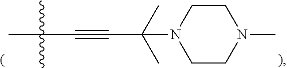 [SMILES: *C#CC(C)(C)N1CCN(C)CC1]

3-methyl-3-amino-1-butyn-1-yl

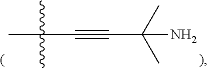 [SMILES: *C#CC(C)(C)N]

methyl-substituted pyrazolyl, hydroxyethyl-substituted pyrazolyl, imidazolyl, pyridinyl; wherein R^{c} represents hydrogen, C₁-C₆ alkyl, C₁-C₆ alkoxy, C₃-C₁₀ cycloalkyl, hydroxy-substituted C₁-C₆ alkyl, C₃-C₆ heterocycloalkyl-substituted C₁-C₆ alkyl, C₃-C₆ heterocycloalkyl-substituted C₁-C₆ alkoxy, C₁-C₆ alkyl-substituted C₃-C₆ heterocycloalkyl, —NR^{a}R^{b}-substituted C₁-C₆ alkyl, or —NR^{a}R^{b}-substituted C₁-C₆ alkoxy. Preferably, R^{c} represents hydrogen, methyl, ethyl, n-propyl, isopropyl, n-butyl, isobutyl, sec-butyl, tert-butyl, hydroxyethyl, methoxyethyl, dimethylaminoethyl, methylsulfonylethyl, hydroxypropyl, methoxypropyl, dimethylaminopropyl, methylsulfonylpropyl, azetidinyl-substituted ethyl, azetidinyl-substituted propyl, pyrrolidinyl-substituted ethyl, pyrrolidinyl-substituted propyl, piperidinyl-substituted ethyl, piperidinyl-substituted propyl, 4-methylpiperidinyl-substituted methyl

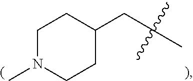 [SMILES: *CC1CCN(C)CC1]

### N-methylpiperazinyl

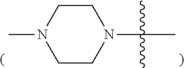 [SMILES: *N1CCN(C)CC1]

substituted ethyl, morpholinyl

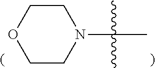 [SMILES: *N1CCOCC1]

substituted ethyl, N-methylpiperazinyl

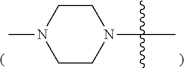 [SMILES: *N1CCN(C)CC1]

substituted propyl, morpholinyl

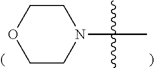 [SMILES: *N1CCOCC1]

substituted propyl, 3-methylpiperidinyl

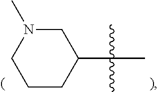 [SMILES: *C1CCCN(C)C1]

4-methylpiperidinyl

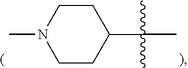 [SMILES: *C1CCN(C)CC1]

3-methylpyrrolidinyl

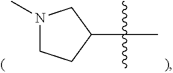 [SMILES: *C1CCN(C)C1]

3-methylazetidinyl

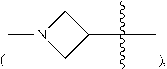 [SMILES: *C1CN(C)C1]

oxetanyl, tetrahydrofuranyl, tetrahydropyranyl; and more preferably, R^{c} represents methyl.

In some more preferred embodiments, R³ represents

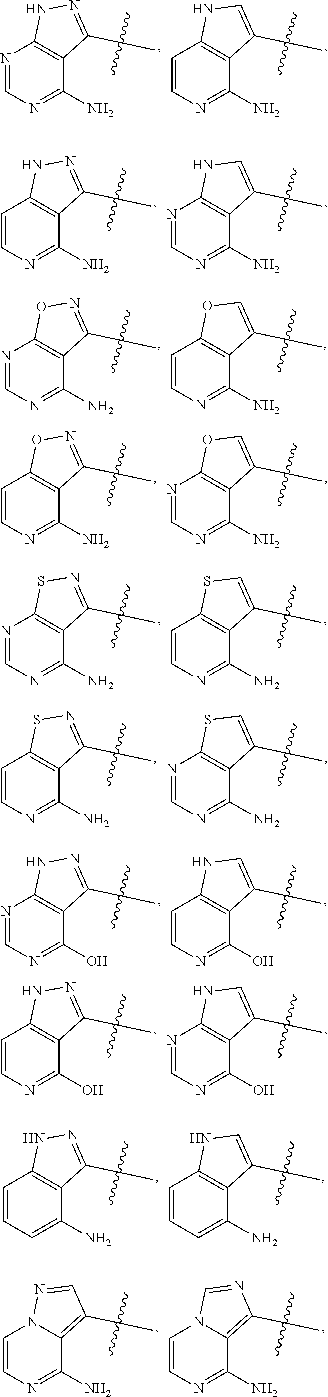

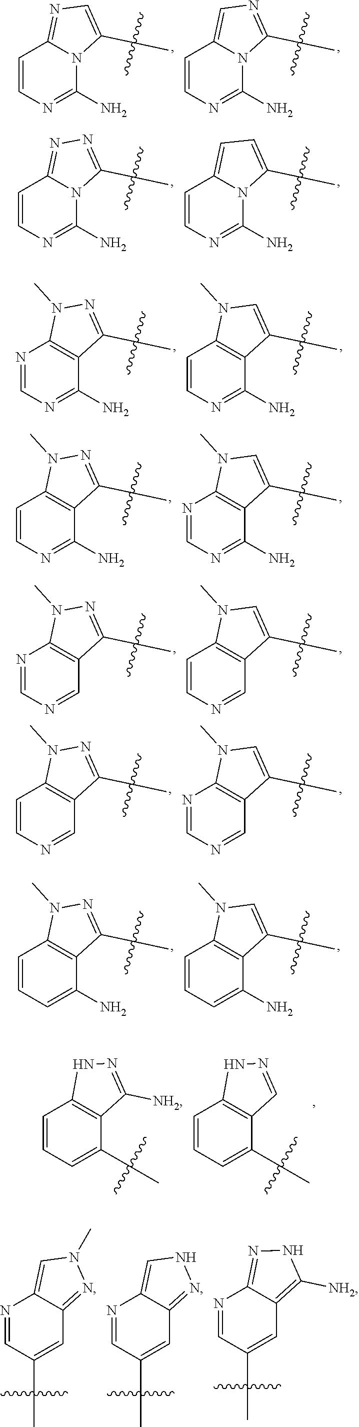

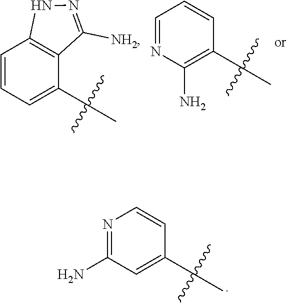

In some more preferred embodiments, R³ represents

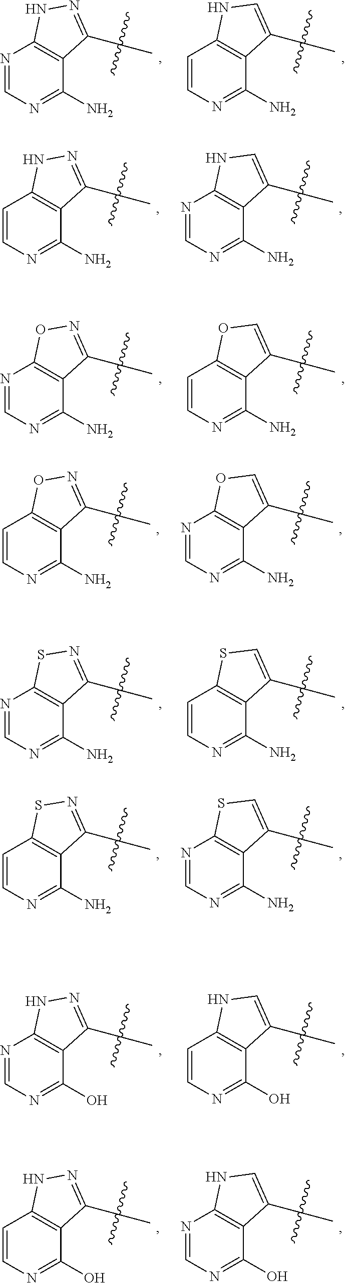

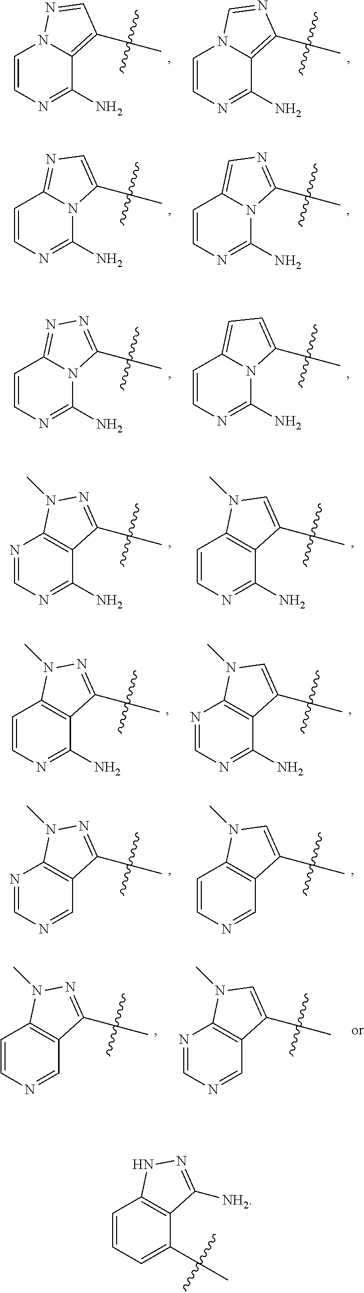

In some preferred embodiments, the compound is not

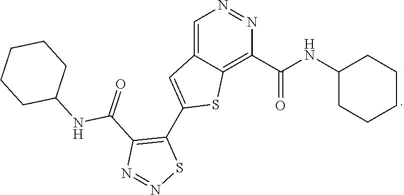 [SMILES: O=C(NC1CCCCC1)c1nnsc1-c1cc2cnnc(C(=O)NC3CCCCC3)c2s1]

In some preferred embodiments, the compound has a structure represented by Formula (I),

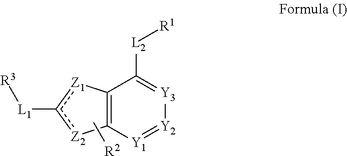

wherein the dashed line represents the absence or presence of a bond, with the proviso that the two dashed lines are not simultaneously absent nor simultaneously bonds;
Z₁ represents S, O or C;
Z₂ represents S, O or C;
Z₁ and Z₂ are not both C;
L₁ represents absent, —(CR^{a}R^{b})ₒ—, —O—, —S—, —S(O)—, —S(O)₂—, —C(O)— or —NR^{a};
L₂ represents —(CR^{a}R^{b})ₒ—, —CR^{a}R^{b}O—, —OCR^{a}R^{b}—, —O—, —C(O)—, —NR^{a}—, —S(O)₂NR^{a}—, —OC(O)—, —C(O)O—, —NR^{a}S(O)₂—, —CR^{a}R^{b}NR^{a}—, —NR^{a}CR^{a}R—, —NR^{a}C(O)NR—, —NR^{a}C(O)N^{b}CR^{a}R—, —NR^{a}C(O)O—, —NR^{a}C(O)— or —C(O)NR^{a}—;
o at each occurrence independently represents an integer from 1 to 20;
Y₁ represents CR^{Y1} or N;
Y₂ represents CR^{Y2} or N;
Y₃ represents CR^{Y3} or N;
R¹ represents substituted or unsubstituted C₁-C₆ alkyl, substituted or unsubstituted C₆-C₁₀ cycloalkenyl, substituted or unsubstituted 4-10 membered heterocycloalkyl, substituted or unsubstituted 6-10 membered heterocycloalkenyl, substituted or unsubstituted C₆-C₁₀ aryl, or substituted or unsubstituted 5-10 membered heteroaryl;
R² represents hydrogen, deuterium, halogen, hydroxy, cyano, —NR^{a}R^{b}, substituted or unsubstituted C₂-C₆ alkenyl, or substituted or unsubstituted C₂-C₆ alkynyl, substituted or unsubstituted C₁-C₆ alkyl, substituted or unsubstituted C₁-C₆ alkoxy, substituted or unsubstituted C₃-C₁₀ cycloalkyl;
R³ represents optionally substituted or unsubstituted heteroaryl;
wherein, R^{Y1}, R^{Y2} and R^{Y3} each independently represent hydrogen, deuterium, halogen, cyano, nitro, substituted or unsubstituted C₁-C₆ alkyl, substituted or unsubstituted C₁-C₆ alkoxy, substituted or unsubstituted C₂-C₆ alkenyl, substituted or unsubstituted C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, substituted or unsubstituted C₃-C₆ cycloalkyl, substituted or unsubstituted C₃-C₆ heterocycloalkyl, substituted or unsubstituted C₆-C₁₀ aryl, or substituted or unsubstituted 5-10 membered heteroaryl;
wherein R^{a} and R^{b} each independently represent hydrogen, deuterium, halogen, cyano, hydroxy, amino, substituted or unsubstituted C₁-C₆ alkyl, substituted or unsubstituted C₁-C₆ alkoxy, or substituted or unsubstituted C₃-C₁₀ cycloalkyl; or R^{a} and R^{b} together with the atom which they are attached to form a 3-6 membered ring, and the said ring optionally contains 0, 1, or 2 heteroatoms selected from O, N, S, or P, and further, the said ring may optionally substituted by substituents selected from halogen, hydroxy, or C₁-C₆ alkyl.

In some preferred embodiments, the compound is selected from any one of the following:

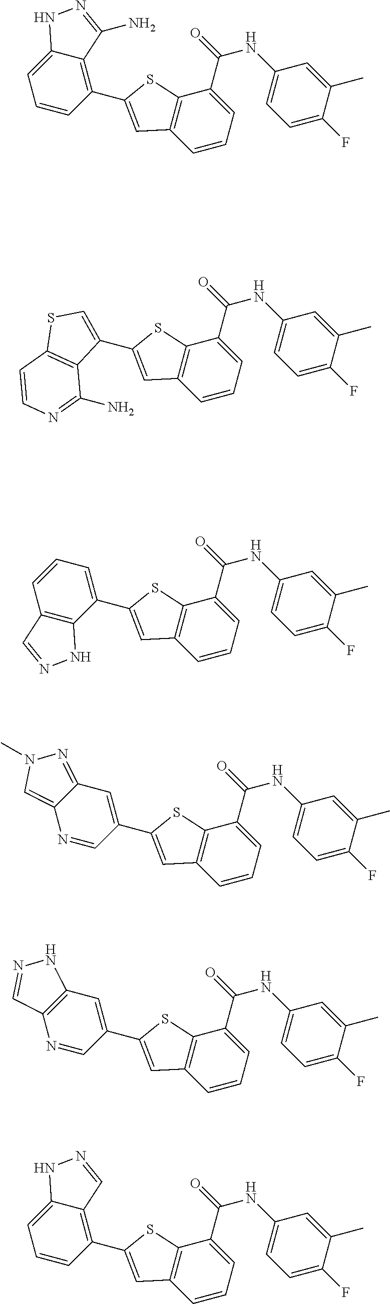

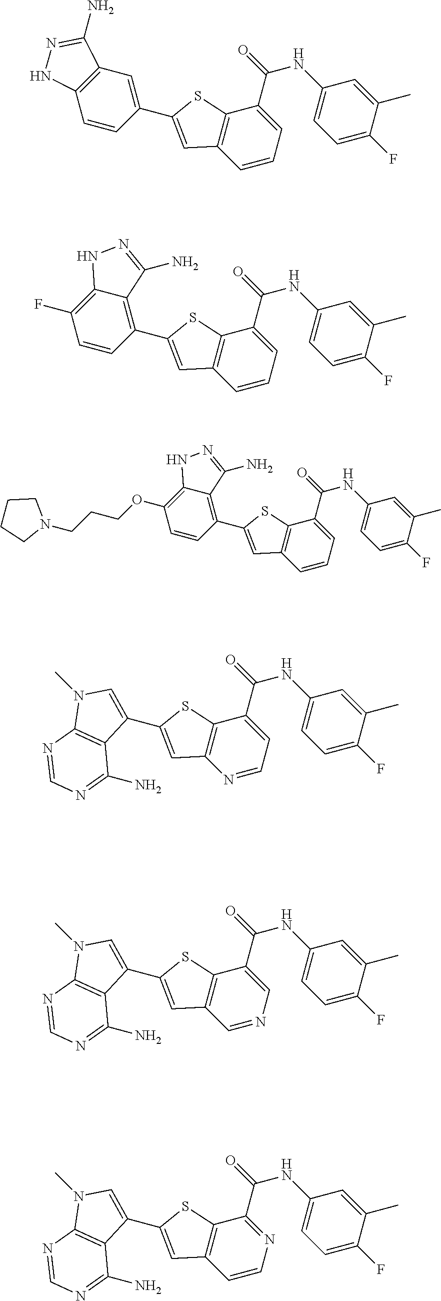 [SMILES: Cc1cc(NC(=O)c2cccc3cc(-c4ccc5[nH]nc(N)c5c4)sc23)ccc1F ; Cc1cc(NC(=O)c2cccc3cc(-c4ccc(F)c5[nH]nc(N)c45)sc23)ccc1F ; Cc1cc(NC(=O)c2cccc3cc(-c4ccc(OCCCN5CCCC5)c5[nH]nc(N)c45)sc23)ccc1F ; Cc1cc(NC(=O)c2ccnc3cc(-c4cn(C)c5ncnc(N)c45)sc23)ccc1F ; Cc1cc(NC(=O)c2cncc3cc(-c4cn(C)c5ncnc(N)c45)sc23)ccc1F ; Cc1cc(NC(=O)c2nccc3cc(-c4cn(C)c5ncnc(N)c45)sc23)ccc1F]

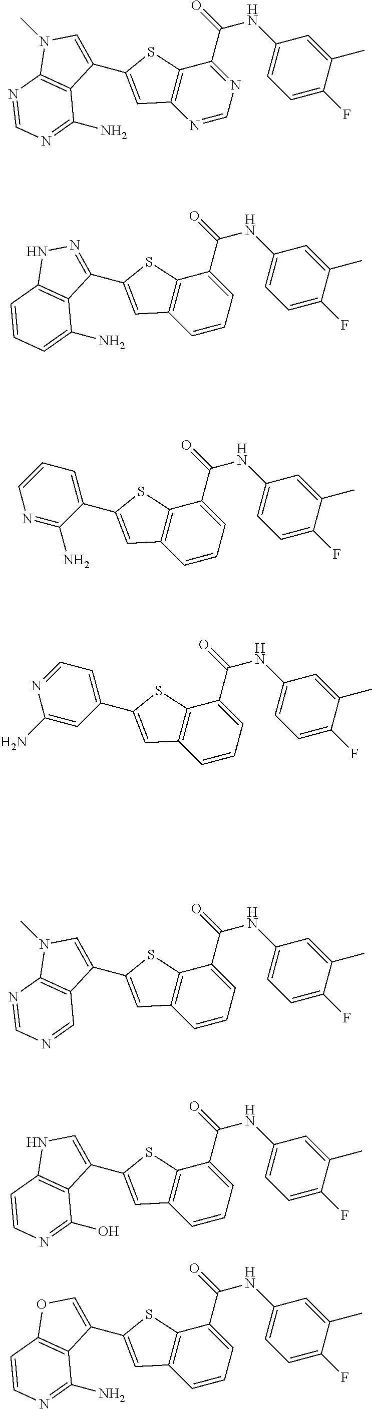

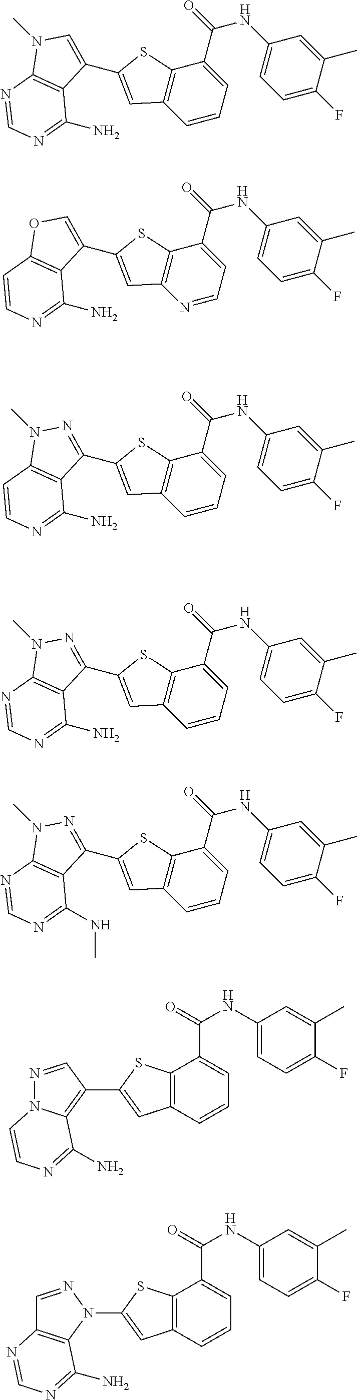

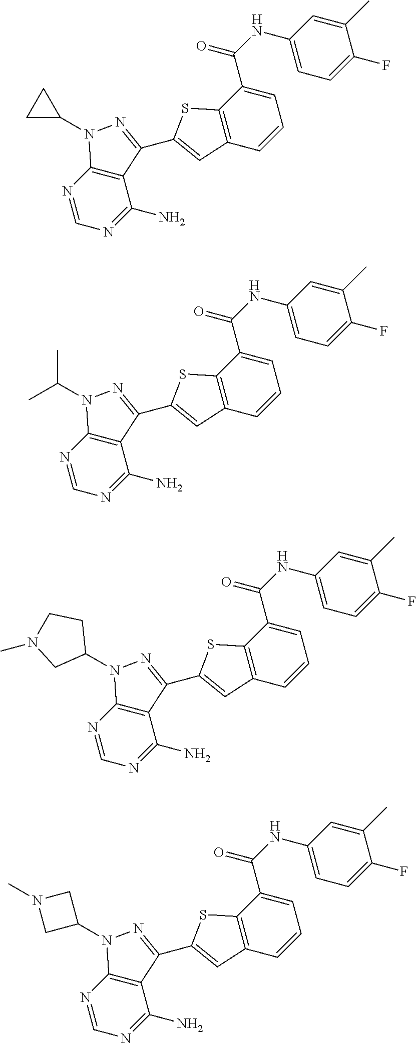 [SMILES: Cc1cc(NC(=O)c2cccc3cc(-c4nn(C5CC5)c5ncnc(N)c45)sc23)ccc1F ; Cc1cc(NC(=O)c2cccc3cc(-c4nn(C(C)C)c5ncnc(N)c45)sc23)ccc1F ; Cc1cc(NC(=O)c2cccc3cc(-c4nn(C5CCN(C)C5)c5ncnc(N)c45)sc23)ccc1F ; Cc1cc(NC(=O)c2cccc3cc(-c4nn(C5CN(C)C5)c5ncnc(N)c45)sc23)ccc1F]

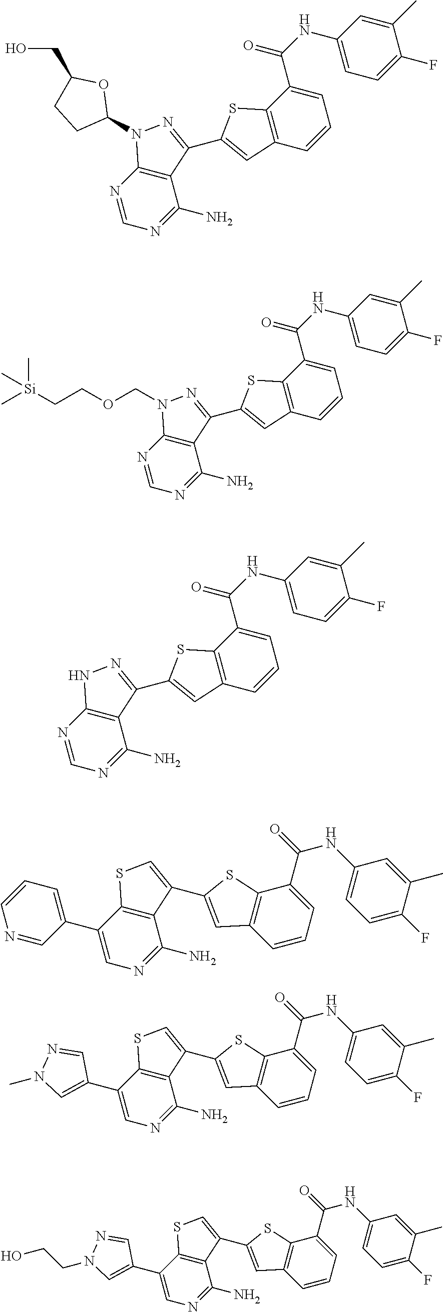

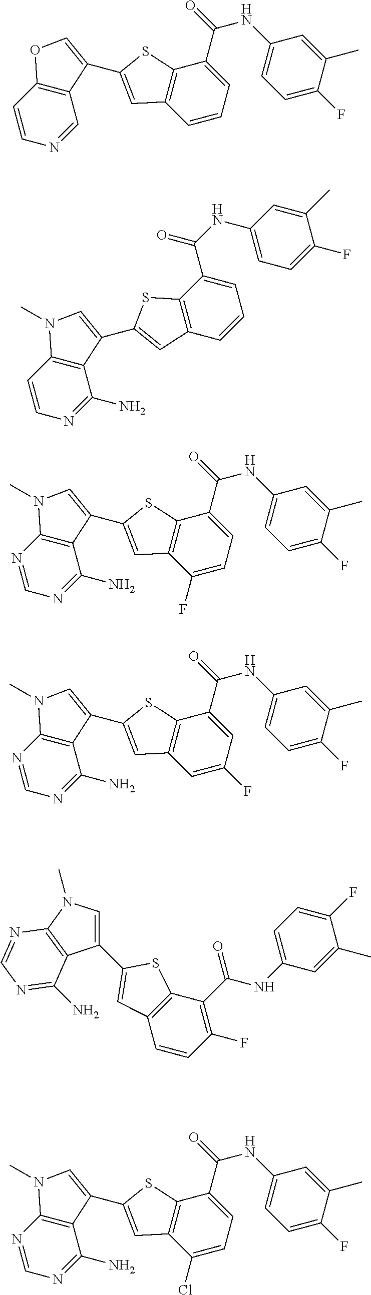

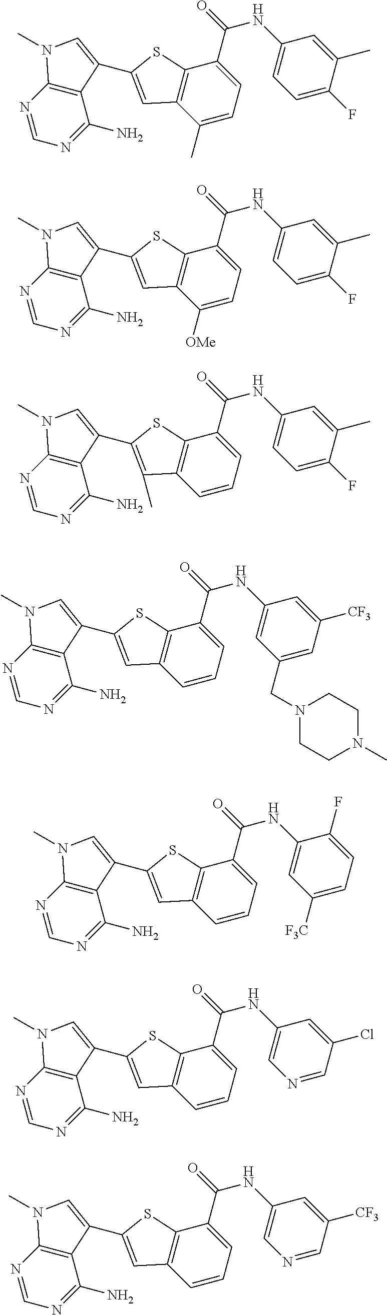

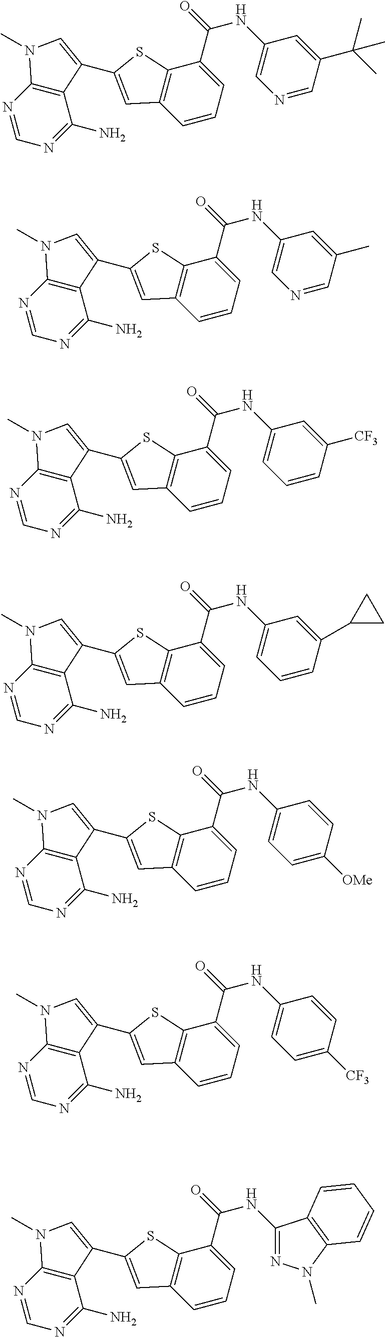

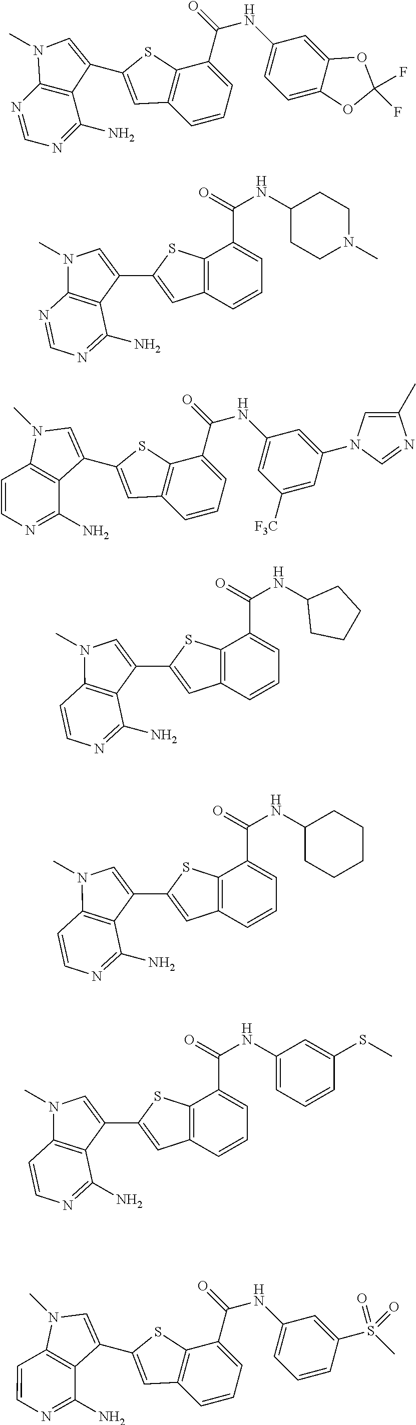

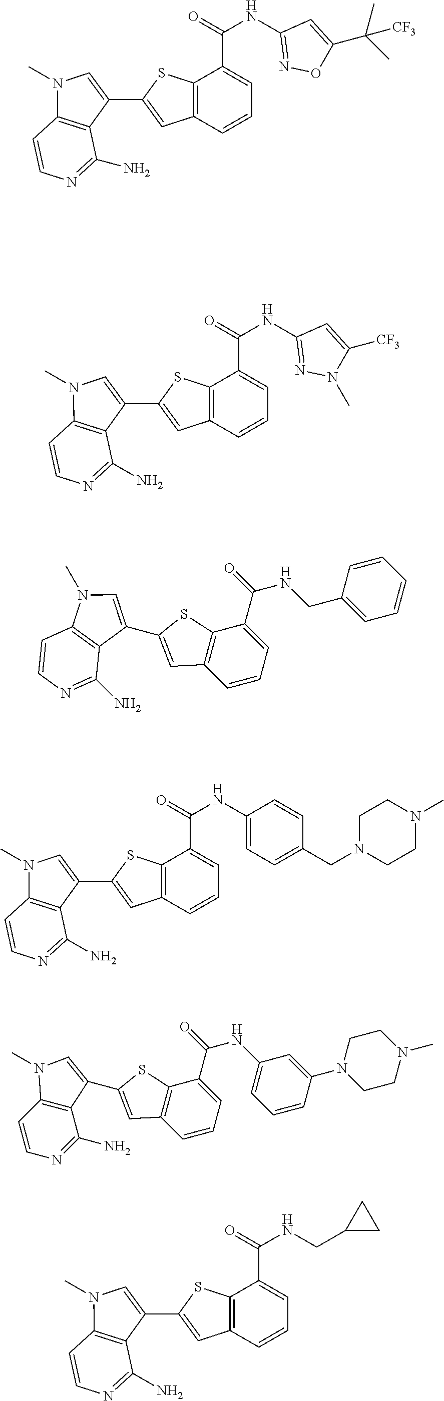

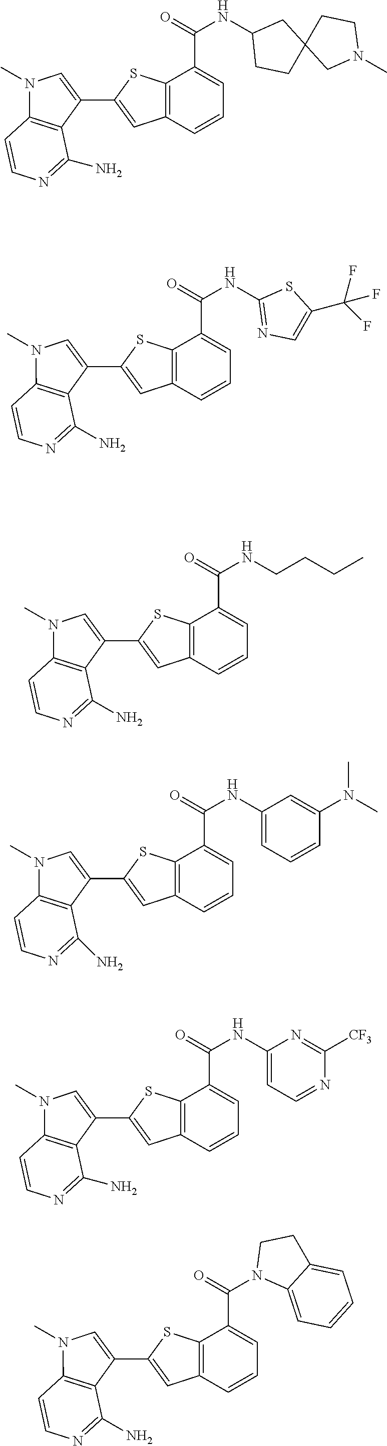

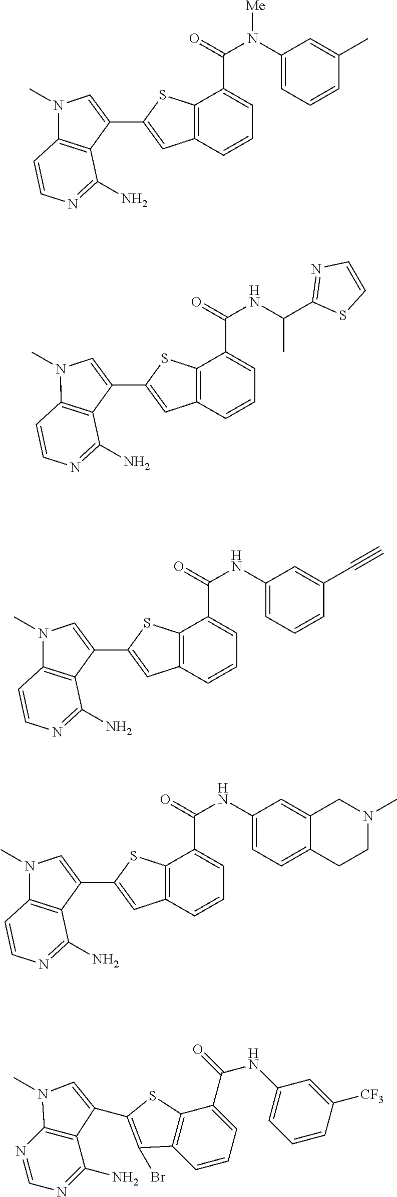

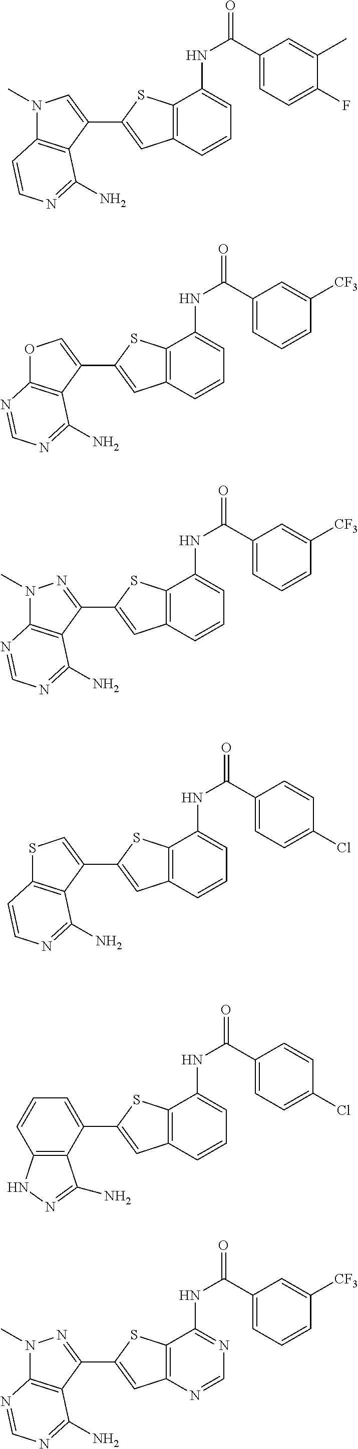 [SMILES: Cc1cc(C(=O)Nc2cccc3cc(-c4cn(C)c5ccnc(N)c45)sc23)ccc1F ; Nc1ncnc2occ(-c3cc4cccc(NC(=O)c5cccc(C(F)(F)F)c5)c4s3)c12 ; Cn1nc(-c2cc3cccc(NC(=O)c4cccc(C(F)(F)F)c4)c3s2)c2c(N)ncnc21 ; Nc1nccc2scc(-c3cc4cccc(NC(=O)c5ccc(Cl)cc5)c4s3)c12 ; Nc1n[nH]c2cccc(-c3cc4cccc(NC(=O)c5ccc(Cl)cc5)c4s3)c12 ; Cn1nc(-c2cc3ncnc(NC(=O)c4cccc(C(F)(F)F)c4)c3s2)c2c(N)ncnc21]

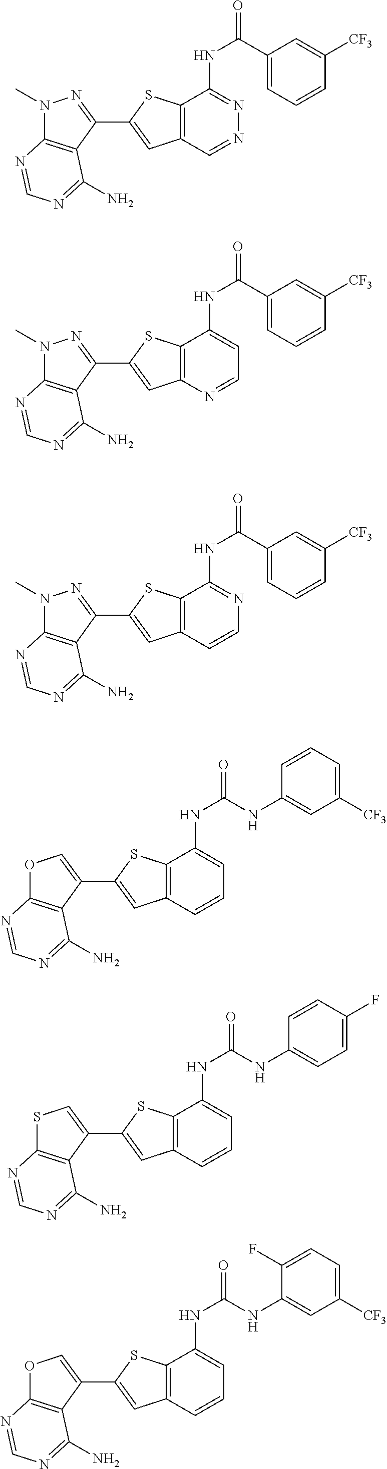 [SMILES: Cn1nc(-c2cc3cnnc(NC(=O)c4cccc(C(F)(F)F)c4)c3s2)c2c(N)ncnc21 ; Cn1nc(-c2cc3nccc(NC(=O)c4cccc(C(F)(F)F)c4)c3s2)c2c(N)ncnc21 ; Cn1nc(-c2cc3ccnc(NC(=O)c4cccc(C(F)(F)F)c4)c3s2)c2c(N)ncnc21 ; Nc1ncnc2occ(-c3cc4cccc(NC(=O)Nc5cccc(C(F)(F)F)c5)c4s3)c12 ; Nc1ncnc2scc(-c3cc4cccc(NC(=O)Nc5ccc(F)cc5)c4s3)c12 ; Nc1ncnc2occ(-c3cc4cccc(NC(=O)Nc5cc(C(F)(F)F)ccc5F)c4s3)c12]

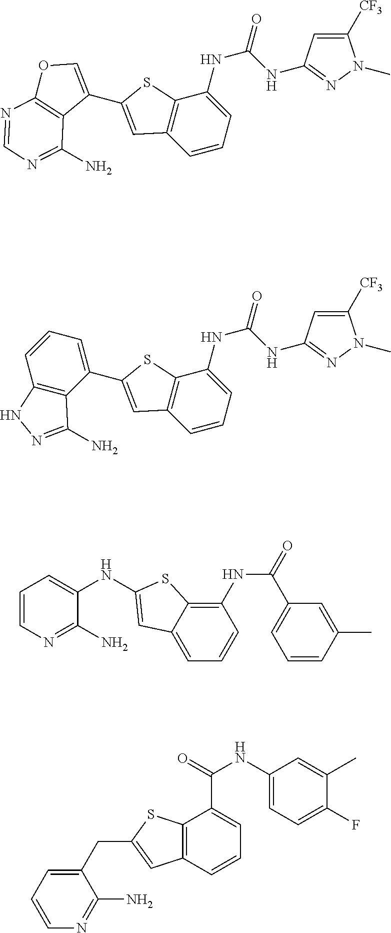 [SMILES: Cn1nc(NC(=O)Nc2cccc3cc(-c4coc5ncnc(N)c45)sc23)cc1C(F)(F)F ; Cn1nc(NC(=O)Nc2cccc3cc(-c4cccc5[nH]nc(N)c45)sc23)cc1C(F)(F)F ; Cc1cccc(C(=O)Nc2cccc3cc(Nc4cccnc4N)sc23)c1 ; Cc1cc(NC(=O)c2cccc3cc(Cc4cccnc4N)sc23)ccc1F]

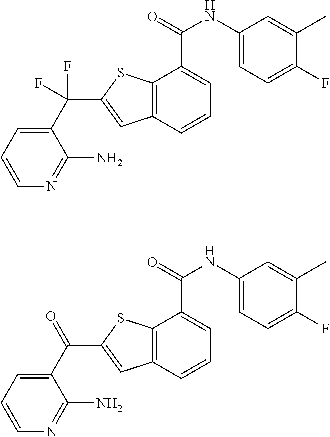 [SMILES: Cc1cc(NC(=O)c2cccc3cc(C(F)(F)c4cccnc4N)sc23)ccc1F ; Cc1cc(NC(=O)c2cccc3cc(C(=O)c4cccnc4N)sc23)ccc1F]

Another aspect of the present disclosure is directed to a pharmaceutical composition, comprising the above-described compound, or a pharmaceutically acceptable salt thereof, stereoisomer thereof, isotopologue thereof, solvate thereof, or prodrug thereof, and a pharmaceutically acceptable excipient.

Another aspect of the present disclosure is directed to a use of the above-described compound, or a pharmaceutically acceptable salt thereof, stereoisomer thereof, isotopologue thereof, solvate thereof, or prodrug thereof, or the pharmaceutical composition thereof, for:
(i) preparation of a tyrosine kinase inhibitor; and/or
(ii) inhibition of tyrosine kinase activity.

In some preferred embodiments, the compound, or a pharmaceutically acceptable salt thereof, stereoisomer thereof, isotopologue thereof, solvate thereof, or prodrug thereof, or the pharmaceutical composition thereof, simultaneously inhibits at least two, preferably three, and more preferably four, selected from the group consisting of CSF-1R, PDGFRα, VEGFR2, and DDR1.

In some embodiments, the (ii) inhibition of tyrosine kinase activity is for in vitro non-therapeutic use, for example, as a scientific research reagent.

Another aspect of the present disclosure is directed to a method of inhibiting tyrosine kinase activity in a patient in need thereof, comprising the step of: administering to the patient in need thereof a therapeutically effective amount of the above-described compound, or a pharmaceutically acceptable salt thereof, stereoisomer thereof, isotopologue thereof, solvate thereof, or prodrug thereof, or the pharmaceutical composition thereof.

Another aspect of the present disclosure is directed to a method for preventing, alleviating, and/or treating a disease associated with overactivation of tyrosine kinase, comprising the step of: administering to a patient in need thereof a therapeutically effective amount of the above-described compound, or a pharmaceutically acceptable salt thereof, stereoisomer thereof, isotopologue thereof, solvate thereof, or prodrug thereof, or the pharmaceutical composition thereof.

### EXAMPLES

The present disclosure provides compounds of Formula (I) that are active against receptor protein tyrosine kinases (RPTKs), as well as their pharmaceutically acceptable salts, stereoisomers, and isotopologues. The compounds of the present disclosure exhibit high biological activity and may be utilized for treating diseases or disorders associated with the target. Based on the foregoing description of the present disclosure, and by applying common technical knowledge and conventional means in the art, other various forms of modifications, substitutions, or alterations may be made without departing from the essential technical spirit of the present disclosure as described above.

### I. DEFINITIONS

Unless otherwise stated, the definitions of groups and terms specified in the specification and claims of the present application, including definitions of examples, exemplary definitions, preferred definitions, definitions recorded in the table, definitions of specific compounds in the embodiments, and the like, can be arbitrarily combined and combined with each other. Such a combination and a combined group definition and compound structure should fall within the scope of the description of the present application.

Unless otherwise defined, the technical terms used herein have the same meaning as commonly understood by a person skilled in the art to which the subject matter belongs. Unless otherwise stated, all patents, patent applications, and publications cited herein are incorporated herein by reference in their entirety. If there are a plurality of definitions to the terms herein, the definition of the present chapter is defined.

It is to be understood that both the foregoing general description and the following detailed description are exemplary and are for explanation only and are not intended to limit the subject matter of the present disclosure. In the present application, unless otherwise specifically stated, the singular also includes the plural. It must be noted that the singular forms used in this specification and the claims include the plural forms of the recited thing, unless expressly stated otherwise herein. It should also be noted that, unless otherwise stated, “or”, “or” means “ and/or”. In addition, the terms “comprising” and other forms, such as “comprising”, “containing”, and “containing” are not intended to be limiting.

Unless otherwise specified, conventional methods within the art, such as mass spectrometry, NMR, IR and UV/VIS spectroscopy, and pharmacological methods, are employed. Unless specifically defined, the terms used herein to analyze chemical, organic synthetic chemistry, and related descriptions of drugs and pharmaceutical chemistry are known in the art. Standard techniques may be used in chemical synthesis, chemical analysis, drug preparation, formulation and delivery, and in the treatment of patients. For example, the reaction and purification may be carried out using a manufacturer's description of the kit, or in a manner well known in the art or a description of the invention. The techniques and methods described above may generally be implemented in accordance with conventional methods well known in the art, in accordance with a number of summaries cited and discussed in this specification and in more specific documents. In the present specification, groups and substituents thereof may be selected by those skilled in the art to provide stable structural moieties and compounds.

Unless otherwise indicated, the compounds of the present disclosure shall be construed to include, in addition to the specific structure of the compound, its pharmaceutically acceptable salts, stereoisomers, isotopologues (e.g., deuterated derivatives), solvates, hydrates, prodrugs, and metabolites; that is to say, the pharmaceutically acceptable salts, stereoisomers, isotopologues, solvates, hydrates, prodrugs, and metabolites of the compound also fall within the scope of protection of the compound.

The compounds of the present disclosure may be asymmetric, e.g., having one or more stereoisomers. Unless otherwise specified, all stereoisomers are encompassed, such as enantiomers and diastereomers. The compounds of the present disclosure containing asymmetric carbon atoms can be isolated in optically active pure form or in racemic form. The optically active pure form can be obtained by resolution of the racemic mixture, or by synthesis using chiral starting materials or chiral reagents. Racemates, diastereomers, and enantiomers are all encompassed within the scope of the present disclosure.

In the present disclosure,

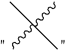

refers to the position at which a substituent is bonded.

In the present disclosure, a numerical range refers to each individual integer within the given range. For example, “C₁₋₆” means that the group can have 1 carbon atom, 2 carbon atoms, 3 carbon atoms, 4 carbon atoms, 5 carbon atoms, or 6 carbon atoms; “C₁₋₃” means that the group can have 1 carbon atom, 2 carbon atoms, or 3 carbon atoms.

The term “optional” or “optionally” means that the subsequently described event or circumstance may or may not occur, and that the description includes instances where said event or circumstance occurs and instances where it does not.

The term “substituted” or “substitution” means that any one or more hydrogen atoms on a specified atom or group is replaced by a substituent, provided that the valence of the specified atom or group is normal and the resulting compound is stable. When the substituent is an ketone group (which can be used interchangeably with “oxo”, i.e., ═O), it means that two hydrogen atoms are replaced. Unless otherwise specified, the type and number of the substituents can be any that are chemically feasible. In some embodiments, the substituent is selected from the group consisting of: halogen atom, alkyl having 1 to 6 carbon atoms, alkoxy having 1 to 6 carbon atoms, haloalkyl having 1 to 6 carbon atoms, haloalkoxy having 1 to 6 carbon atoms, cyano, alkynyl having 2 to 6 carbon atoms, alkanoyl having 1 to 6 carbon atoms, cycloalkyl having 3 to 7 ring atoms, heteroaryl, aryl, aralkyloxy having 7-10 carbon atoms, aryl carbonyl, aminocarbonyl, alkenyl having 2 to 5 carbon atoms, alkylthio having 1 to 6 carbon atoms, aminosulfinyl, aminosulfonyl, hydroxy, —SF₅, hydroxyalkyl having 1 to 4 carbon atoms, nitro, amino, carboxy, alkoxycarbonyl having 2 to 5 carbon atoms, alkoxyalkyl having 1 to 4 carbon atoms, alkylsulfonyl having 1 to 4 carbon atoms, alkanoylamino having 1 to 4 carbon atoms, alkanoyl(alkyl)amino having 1 to 6 carbon atoms in each of the alkanoyl and alkyl parts, alkanoylaminoalkyl having 1 to 6 carbon atoms in each of the alkanoyl and alkyl parts, alkanoyl(alkyl)aminoalkyl having 1 to 6 carbon atoms in each of the alkanoyl and alkyl parts, alkylsulfonylamino having 1 to 4 carbon atoms, monoalkylaminocarbonyl or dialkylaminocarbonyl having 1 to 6 carbon atoms, monoalkylaminosulfinyl or dialkylaminosulfinyl having 1 to 6 carbon atoms, monoalkylaminosulfonyl or dialkylaminosulfonyl having 1 to 6 carbon atoms, aminoalkyl having 1 to 4 carbon atoms, monoalkylamino or dialkylamino having 1 to 6 carbon atoms, monoalkylaminoalkyl or dialkylaminoalkyl having 1 to 6 carbon atoms in each alkyl part, aralkyl having 7 to 10 carbon atoms, heteroaralkyl having 1 to 4 carbon atoms in the alkyl part, heteroarylalkoxy having 1 to 4 carbon atoms in the alkoxy part, and alkylsulfonamido having 1 to 4 carbon atoms.

Where any variable (e.g., Rⁿ) occurs more than once in the composition or structure of a compound, its definition in each occurrence is independent. Thus, for example, if a group is substituted by one to three R, said group may optionally be substituted by up to three R, and R in each occurrence has independent selections. Furthermore, combinations of substituents and/or variants thereof are permissible only if such combinations result in stable compounds.

The term “alkyl” refers to a saturated aliphatic hydrocarbon group, including straight-chain or branched-chain saturated hydrocarbon groups, said hydrocarbon group having the indicated number of carbon atoms. For example, the term “C₁₋₆ alkyl” includes C₁ alkyl, C₂ alkyl, C₃ alkyl, C₄ alkyl, C₅ alkyl, and C₆ alkyl. Examples include, but are not limited to, methyl, ethyl, n-propyl, isopropyl, n-butyl, isobutyl, tert-butyl, n-pentyl, 2-pentyl, 3-pentyl, n-hexyl, 2-hexyl, 3-hexyl, etc. It may be divalent, e.g., methylene, ethylene.

The term “haloalkyl” refers to an alkyl group as defined above, wherein one or more hydrogen atoms are replaced by halogen atoms. The term “halo-” refers to the substitution of at least one hydrogen atom in a molecule or group by a halogen/halogen atom.

The term “alkylene” by itself or as part of another substituent refers to a divalent group derived from an alkyl group, examples of which include, but are not limited to, —CH₂CH₂CH₂CH₂—, —CH₂CH═CHCH₂—, —CH₂C—CCH₂—, —CH₂CH₂CH(CH₂CH₂CH₃)CH₂—. An alkyl (or alkylene) group typically has from 1 to 24 carbon atoms, with groups having 10 or fewer carbon atoms being preferred in the present disclosure. A “lower alkyl” or “lower alkylene” refers to a shorter-chain alkyl or alkylene group, typically having eight or fewer carbon atoms. Said alkylene is optionally substituted by one or more halogen atoms.

The term “alkynyl” refers to a carbon chain containing at least one carbon-carbon triple bond, which may be linear or branched, or a combination thereof. Examples of alkynyl include ethynyl, propargyl, 3-methyl-1-pentynyl, 2-heptynyl, etc. Said alkynyl is optionally substituted by one or more halogen atoms.

The term “cycloalkyl” refers to a monocyclic or bicyclic saturated carbocyclic ring, each having from 3 to 10 carbon atoms. A “fused analog” of a cycloalkyl refers to a monocyclic ring fused with an aryl or heteroaryl group, wherein the point of attachment is on the non-aromatic portion. Examples of cycloalkyl and its fused analogs include cyclopropyl, cyclobutyl, cyclopentyl, cyclohexyl, tetrahydronaphthyl, decahydronaphthyl, dihydroindenyl, etc. Said cycloalkyl is optionally substituted by one or more halogen atoms. Furthermore, the term “cycloalkyl” in the present disclosure includes bridged ring systems and spiro ring systems. For example, instances of the term “C₃₋₆ cycloalkyl” include cyclopropyl, cyclobutyl, cyclopentyl, cyclohexyl.

The term “cycloalkenyl” refers to a monocyclic or bicyclic unsaturated carbocyclic ring containing one or more unsaturated C—C double bonds within the ring.

The term “cycloalkoxy” refers to a cycloalkyl group as defined above attached to an oxygen atom, such as cyclopropoxy.

The term “haloalkoxy” refers to an alkoxy group as defined above wherein one or more hydrogen atoms are substituted by halogen.

The term “heteroalkyl”, by itself or in combination with another term, refers to a stable straight-chain, branched-chain, or cyclic hydrocarbon radical, or a combination thereof, consisting of at least one carbon atom and at least one heteroatom selected from O, N, P, Si, and S, wherein the nitrogen, phosphorus, or sulfur atom(s) may optionally be oxidized and the nitrogen atom(s) may optionally be quaternized. The heteroatoms 0, N, P, S, and Si may be placed at any position of the heteroalkyl or at the position where the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and cyano. A maximum of two or three heteroatoms may be consecutive. For example, —CH₂—NH—OCH₃ and —CH₂—O—Si(CH₃)₃. Similarly, the term “heteroalkylene”, by itself or in combination with other terms, refers to a divalent radical derived from a heteroalkyl group, including, for example, but not limited to, —CH₂—CH₂—S—CH₂—CH₂— and —CH₂—S—CH₂—CH₂—NH—CH₂—. For heteroalkylene, the heteroatom(s) may reside at either or both ends of the chain (e.g., alkyleneoxy, alkylenedioxy, alkyleneamino, alkylenediamino, etc.). Furthermore, for alkylene and heteroalkylene linking groups, the writing direction of the group's formula does not dictate the orientation of the linking group. For example, the formula —C(O)OR′— denotes both —C(O)OR′— and —R′OC(O)—. As mentioned above, the heteroalkyl groups as used herein include those groups that are attached to the remainder of the molecule through a heteroatom, such as —C(O)R′, —C(O)NR′, —NR′R¹¹, —OR′, —SR′, and/or —SO₂R′. Where “heteroalkyl” is recited and followed by recitations of specific heteroalkyl groups, such as —NR′R¹¹, it is understood that the terms heteroalkyl and —NR′R¹¹ are not redundant or mutually exclusive. Rather, these specific heteroalkyl groups are cited for clarity.

Thus, the term “heteroalkyl” should not be interpreted herein as excluding specific heteroalkyl groups such as —NR′R″.

The term “alkoxy” may be straight-chain, branched, or cyclic. The number of carbon atoms in the alkoxy group is not particularly limited but is preferably 1 to 20. Specific examples thereof include methoxy, ethoxy, n-propoxy, isopropoxy, i-propyloxy, n-butoxy, isobutoxy, tert-butoxy, sec-butoxy, n-pentyloxy, neopentyloxy, isopentyloxy, n-hexyloxy, 3,3-dimethylbutoxy, 2-ethylbutoxy, n-octyloxy, n-nonyloxy, n-decyloxy, and the like, but are not limited thereto.

The term “carbonyl” or “carboxy” encompasses structures wherein the carbon of a compound or fragment is connected to an oxygen atom by a double bond. Examples of moieties containing a carbonyl group include aldehydes, ketones, carboxylic acids, amides, esters, anhydrides, and the like.

The term “acyl” refers to a carbonyl structure wherein the carbon atom of the carbonyl is connected to hydrogen (i.e., formyl), an aliphatic group (C₁-C₆ alkyl, C₁-C₆ alkenyl, C₁-C₆ alkynyl, e.g., acetyl), a cycloalkyl group (C₃-C₈ cycloalkyl), a heterocyclic group (C₃-C₈ heterocycloalkyl and C₅-C₆ heteroaryl), or an aryl group (C₆ aryl, e.g., benzoyl). The acyl group may be unsubstituted or substituted (e.g., salicyloyl).

In the present disclosure, examples of “halogen” or “halo atom” may include fluorine, chlorine, bromine, or iodine.

In the present disclosure, “amino” denotes —NH₂. When one or more hydrogens in the amino group are substituted by an alkyl group, an “alkylamino” is formed; for example, substitution of one hydrogen by methyl forms —N(CH₃)H, and substitution of two hydrogens by methyl forms —N(CH₃)₂.

In the present disclosure, the term “aryl” refers to an all-carbon monocyclic or fused polycyclic aromatic ring group possessing a conjugated π-electron system, which is derived by removing one hydrogen atom from a single carbon atom of the parent aromatic ring system. It includes bicyclic groups wherein a saturated or partially unsaturated ring, or an aromatic carbocyclic ring, is fused to the aromatic ring. Specific examples thereof include, but are not limited to, phenyl or naphthyl. When an aryl group is substituted, two adjacent substituents may be bonded to each other to form a ring; in one embodiment, adjacent methoxy groups on a phenyl ring are bonded to each other to form

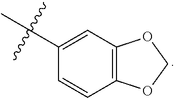 [SMILES: *c1ccc2c(c1)OCO2]

The term “heterocycle” or “heterocyclic ring” refers to a saturated or unsaturated non-aromatic system having ring carbon atoms and no less than one ring heteroatom, wherein the heteroatom(s) are independently selected from nitrogen, sulfur, or oxygen atoms. In heterocyclic groups containing one or more nitrogen atoms, the point of attachment can be a carbon or nitrogen atom, provided valence permits. The heterocycle can be a monocyclic or polycyclic system, e.g., bicyclic, wherein two or more rings are fused, bridged, or spiro-fused, with at least one ring containing one or more heteroatoms. In a spiro heterocycle, two distinct rings share a single atom; an example of a spiro heterocycle is azaspiropentane, but it is not limited thereto. In one embodiment, the heterocycle is a 4- to 12-membered heterocycle, preferably a 4- to 10-membered heterocycle.

In the present disclosure, the term “heterocycloalkyl” refers to a saturated non-aromatic substituent having ring carbon atoms and at least one ring heteroatom. In a preferred embodiment, the heterocycloalkyl is a 4- to 12-membered cycloalkyl containing at least one ring heteroatom selected from nitrogen, oxygen, or sulfur. Specific examples of heterocycloalkyl include, but are not limited to, piperidinyl or tetrahydropyrrolyl (pyrrolidinyl).

The term “heterocycloalkenyl” refers to an unsaturated non-aromatic substituent having ring carbon atoms and at least one ring heteroatom, wherein the ring contains one or more unsaturated C—C double bonds, illustratively including, but not limited to,

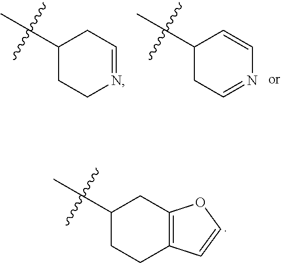 [SMILES: *C1CC=NCC1 ; *C1C=CN=CC1 ; "or" ; *C1CCc2ccoc2C1]

In the present disclosure, the term “heteroaryl” refers to an aryl group containing at least one heteroatom independently selected from nitrogen, oxygen, and sulfur. The heteroaryl group can be monocyclic or a polycyclic system, e.g., bicyclic, wherein two or more rings are fused, bridged, or spiro-fused, with at least one ring containing one or more heteroatoms. Specific examples of heteroaryl include, but are not limited to, pyridyl, thienyl, imidazolyl, pyrimidinyl, furyl, pyrazinyl, thiazolyl, quinolinyl, isoquinolinyl, indolyl, benzimidazolyl, imidazopyridinyl, benzofuranyl, pyridazinyl, isoindolyl, pyridonyl, etc. When a heteroaryl group is substituted, two adjacent substituents may be bonded to each other to form a ring; in one embodiment, adjacent hydroxyl and ethyl groups on a pyridyl ring are bonded to each other to form

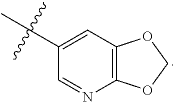 [SMILES: *c1cnc2c(c1)OCO2]

The term “prodrug” refers to a compound that is converted in vivo into the parent drug substance. Prodrugs are often used because, under certain circumstances, they may be easier to administer than the parent drug. For example, a prodrug may be bioavailable by oral administration whereas the parent drug is not. A prodrug may also have higher solubility than the parent drug in pharmaceutical compositions. Examples of prodrugs include, but are not limited to, esters of compounds of Formula I (acting as prodrugs) administered to facilitate transport across cell membranes where water solubility is detrimental to mobility, but where, inside the cell, the ester is subsequently metabolically hydrolyzed to the carboxylic acid active substance. Another example of a prodrug is a short peptide (polyamino acid) linked to an acid group, wherein the peptide is metabolized to release the active moiety.

### Pharmaceutical or Pharmaceutical Composition

The term “pharmaceutically acceptable” refers to those compounds, materials, compositions, and/or dosage forms which are, within the scope of sound medical judgment, suitable for contact with the tissues of humans and animals without undue toxicity, irritation, allergic reactions, or other problems or complications commensurate with a reasonable benefit/risk ratio.

The term “pharmaceutically acceptable salt” refers to a salt that retains the biological effectiveness of the free acid or base of the specified compound without undesirable biological effects. Examples include acid addition salts (including those derived from organic and inorganic acids) or base addition salts (including those derived from organic and inorganic bases).

The pharmaceutically acceptable salts of the present disclosure can be synthesized from parent compounds containing acid or base moieties by conventional chemical methods. Generally, such salts are prepared by reacting the free acid or base forms of these compounds with a stoichiometrically appropriate amount of the corresponding base or acid in water or an organic solvent, or a mixture thereof.

The pharmaceutical or pharmaceutical composition of the present disclosure may be administered orally, topically, parenterally, or mucosally (e.g., buccally, by inhalation, or rectally) in dosage unit formulations containing conventional non-toxic pharmaceutically acceptable carriers.

For oral administration in tablet or capsule form, the active pharmaceutical ingredient may be combined with non-toxic, pharmaceutically acceptable excipients such as binders (e.g., pregelatinized corn starch, polyvinylpyrrolidone, or hypromellose); fillers (e.g., lactose, sucrose, glucose, mannitol, sorbitol, other reducing and non-reducing sugars, microcrystalline cellulose, calcium sulfate, or dicalcium phosphate); lubricants (e.g., magnesium stearate, talc, or silica, stearic acid, sodium stearyl fumarate, glyceryl behenate, calcium stearate, etc.); disintegrants (e.g., potato starch or sodium starch glycolate); or wetting agents (e.g., sodium lauryl sulfate), coloring and flavoring agents, gelatin, sweeteners, natural and synthetic gums (e.g., acacia, tragacanth, or alginates), buffering salts, carboxymethylcellulose, polyethylene glycol, waxes, etc. For oral administration in liquid form, the pharmaceutical component may be combined with non-toxic, pharmaceutically acceptable inert carriers (e.g., ethanol, glycerol, water), suspending agents (e.g., sorbitol syrup, cellulose derivatives, or hydrogenated edible fats), emulsifying agents (e.g., lecithin or acacia), non-aqueous carriers (e.g., almond oil, oily esters, ethanol, or fractionated vegetable oils), preservatives (e.g., methyl p-hydroxybenzoate, propyl p-hydroxybenzoate, or sorbic acid), etc. Stabilizers such as antioxidants (BHA, BHT, propyl gallate, sodium ascorbate, citric acid) may also be added to stabilize the dosage form.

Tablets containing the active compound may be coated by methods well known in the art. The compositions of the present disclosure containing a compound of Formula I as the active compound may also be incorporated into beads, microspheres, or microcapsules, e.g., constructed from poly(glycolic acid/lactic acid) (PGLA). Liquid preparations for oral administration may take the form of, for example, solutions, syrups, emulsions, or suspensions, or they may be presented as a dry product for reconstitution with water or other suitable vehicles prior to use. Formulations for oral administration may be suitably formulated to achieve controlled or delayed release of the active compound.

The pharmaceutical or pharmaceutical composition of the present disclosure may be delivered parenterally, i.e., by intravenous (i.v.), intracerebroventricular (i.e.v.), subcutaneous (s.c.), intraperitoneal (i.p.), intramuscular (i.m.), subdermal (s.d.), or intradermal (i.d.) administration, via direct injection, e.g., by a rapid bolus or by continuous infusion. Formulations for injection may be presented in unit dosage form, e.g., in ampoules or in multi-dose containers with an added preservative. The compositions may take the form of suspensions, solutions, or emulsions in oily or aqueous vehicles, and may contain formulating agents such as suspending agents, stabilizers, and/or dispersing agents. Alternatively, the active ingredient may be in powder form for reconstitution with a suitable vehicle, such as sterile, pyrogen-free water, prior to use.

The pharmaceutical or pharmaceutical composition of the present disclosure may also be is formulated for rectal administration, e.g., as suppositories or retention enemas (e.g., containing conventional suppository bases such as cocoa butter or other glycerides).

The term “treatment” includes inhibiting, alleviating, preventing, or eliminating one or more symptoms or side effects associated with the disease, disorder, or condition being treated.

The term “effective amount” or “therapeutically effective amount” refers to a dose sufficient to treat, inhibit, or ameliorate one or more symptoms of the disease state being treated, or to otherwise provide a desired pharmacological and/or physiological effect. The precise dosage will vary depending on a variety of factors such as subject-dependent variables (e.g., age, health of the immune system, etc.), the disease or illness, and the treatment administered. The effect of an effective amount can be compared to controls. Such controls are known in the art and discussed herein, and may be, for example, the condition of the subject before administration of the drug or pharmaceutical combination, or without administration, or in the case of a drug combination, the effect of the combination may be compared to the effect of administering only one drug.

The term “pharmaceutical composition” means a composition comprising a compound of the present disclosure or a pharmaceutically acceptable salt thereof, and at least one pharmaceutically acceptable ingredient selected based on the route of administration and the nature of the dosage form, including but not limited to: carriers, diluents, adjuvants, excipients, preservatives, fillers, disintegrants, wetting agents, emulsifying agents, suspending agents, sweetening agents, flavoring agents, perfuming agents, antibacterial agents, antifungal agents, lubricants, dispersing agents, thermosensitive materials, temperature-modulating agents, adhesives, stabilizers, suspending agents, and the like.

### II. EXAMPLES

The present disclosure is further illustrated below with reference to the embodiments. The description of specific exemplary embodiments of the present disclosure is for the purpose of illustration and exemplification. These descriptions are not intended to limit the present disclosure to the precise forms disclosed, and it is obvious that many modifications and variations are possible in light of the teachings of the present disclosure. The selection and description of the exemplary embodiments are to explain the specific principles of the present disclosure and their practical applications, thereby enabling those skilled in the art to implement and utilize the various exemplary embodiments of the present disclosure as well as various choices and modifications thereof.

The experimental methods used in the embodiments hereinafter are conventional methods unless otherwise specified.

The materials, reagents, and the like used in the embodiments hereinafter are all commercially available unless otherwise specified.

### Example 1

### 2-(3-amino-1H-indazol-4-yl)-N-(4-fluoro-3-methylphenyl)benzo[b]thiophene-7-carboxamide (compound 1)

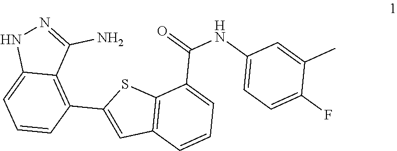 [SMILES: Cc1cc(NC(=O)c2cccc3cc(-c4cccc5[nH]nc(N)c45)sc23)ccc1F]

Compound 1 was prepared by the following steps:

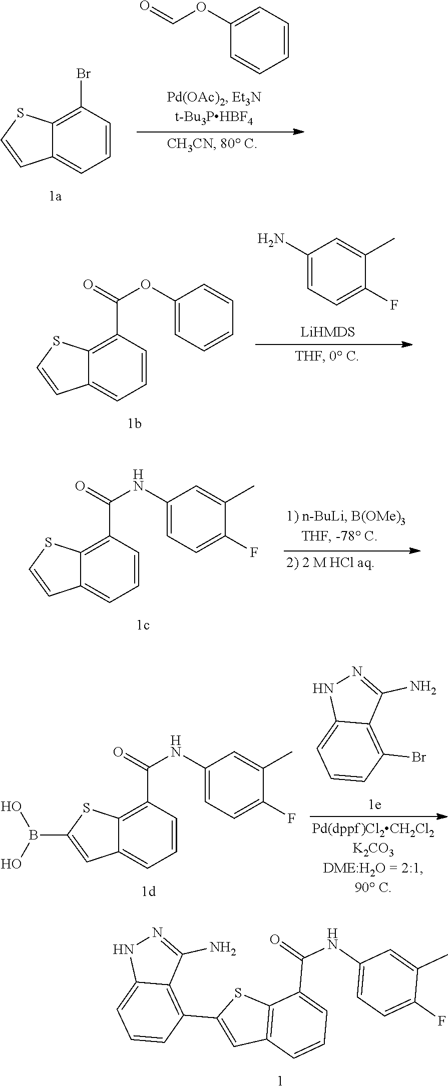

Step 1: To a solution of compound 1a (10.00 g, 47.00 mmol) and t-Bu₃P·HBF₄ (CAS NO.: 131274-22-1) (5.45 g, 18.80 mmol) in CH₃CN (100.0 mL) was added triethylamine (9.57 g, 94.00 mmol, 13.2 mL), phenyl formate (11.60 g, 94.00 mmol, 10.3 mL) and Pd(OAc)₂ (1.06 g, 4.70 mmol). The resultant mixture was degassed with N₂ for three times and stirred at 80° C. After the reaction was completed monitored by TLC, the reaction mixture was concentrated to dryness, and the resulting residue was purified on an ISCO chromatography (mobile phase: petroleum ether/ethyl acetate) to afford compound 1b (8.00 g, yield: 67%). ¹H NMR (400 MHz, DMSO-d₆) δ 8.36-8.26 (m, 2H), 7.93 (d, J=5.5 Hz, 1H), 7.69-7.59 (m, 2H), 7.55-7.47 (m, 2H), 7.41-7.33 (m, 3H).

Step 2: To a stirred solution of compound 1b (2.00 g, 7.90 mmol) and 2-fluoro-5-aminotoluene (1.20 g, 9.50 mmol) in THF (20.0 mL) was added LiHMDS (20.0 mL, 1 M in THF, 19.80 mmol) at 0° C. The reaction was quenched with saturated aq. NH₄Cl (50.0 mL) after completion detected by TLC, added ethyl acetate (50.0 mL), stirred for a while and separated. The organic phase was dried over anhydrous Na₂SO₄, then concentrated to dryness. The resulting residue was purified on an ISCO chromatography (mobile phase: petroleum ether/ethyl acetate) to afford compound 1c (2.05 g, yield: 91%). ¹H NMR (400 MHz, DMSO-d₆) δ 10.43 (s, 1H), 8.22-8.09 (m, 2H), 7.87 (d, J=5.5 Hz, 1H), 7.76 (dd, J=7.1, 2.4 Hz, 1H), 7.65-7.51 (m, 3H), 7.21-7.11 (m, 1H), 2.27 (s, 3H).

Step 3: To a solution of compound 1c (1.00 g, 3.51 mmol) in anhydrous THF (20.0 mL) was slowly added n-BuLi (5.60 mL, 2.5 M in hexane, 14.10 mmol) dropwise at −78° C., followed by addition of trimethyl borate (1.09 g, 10.5 mmol, 1.2 mL) after being stirred for 1 h. The resulting mixture was continued stirred for 1.5 h, then warmed to room temperature. HCl (aq. 2.0 M, 10.0 mL) was added and stirred for 30 min, and then saturated aq. Na₂CO₃ (50.0 mL) was added. The mixture was extracted with ethyl acetate twice, acidified to pH 2-3 with aq. HCl (2.0 M), and extracted with ethyl acetate for three times. The combined organic phases were washed with saturated brine and concentrated to afford crude product 1d (716 mg, yield: 62%). ESI-MS (m/z): 330.1 [M+1]⁺.

Step 4: To a solution of compound 1d (50.0 mg, 0.152 mmol) in a mixed solvent of DME (1.0 mL) and H₂O (0.5 mL) was sequentially added compound 1e (38.6 mg, 0.182 mmol), potassium carbonate (63.0 mg, 0.456 mmol) and Pd(dppf)Cl₂·CH₂Cl₂ (12.4 mg, 0.0152 mmol). The reaction mixture was degassed with N₂ for three times, then stirred at 90° C. for 3 h. After concentration, the residue was added saturated aq. Na₂CO₃ (10.0 mL), and extracted with a mixed solvent of CH₂Cl₂/MeOH (v/v=10:1, 10.0 mL*3). The combined organic phases were washed with saturated brine (30.0 mL), dried over anhydrous Na₂SO₄, filtered, and concentrated. The is resulting residue was purified on an ISCO chromatography (mobile phase: CH₃CN/H₂O) to afford compound 1 as a pale yellow solid (11.2 mg, yield: 19%). ESI-MS (m/z): 417.0 [M+H]⁺. ¹H NMR (400 MHz, DMSO-d₆) δ 10.49 (s, 1H), 8.24 (d, J=7.6 Hz, 1H), 8.16 (d, J=7.8 Hz, 1H), 7.80-7.75 (m, 1H), 7.69 (s, 1H), 7.66-7.59 (m, 2H), 7.44-7.34 (m, 2H), 7.20-7.09 (m, 2H), 2.27 (s, 3H).

### Example 2

### 2-(4-aminothieno[3,2-c]pyridin-3-yl)-N-(4-fluoro-3-methylphenyl)benzo[b]thiophene-7-carboxamide (compound 2)

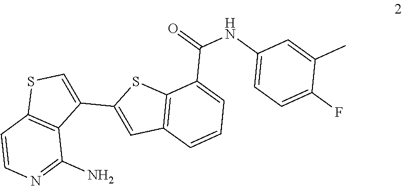 [SMILES: Cc1cc(NC(=O)c2cccc3cc(-c4csc5ccnc(N)c45)sc23)ccc1F]

Compound 2 was prepared by the following step:

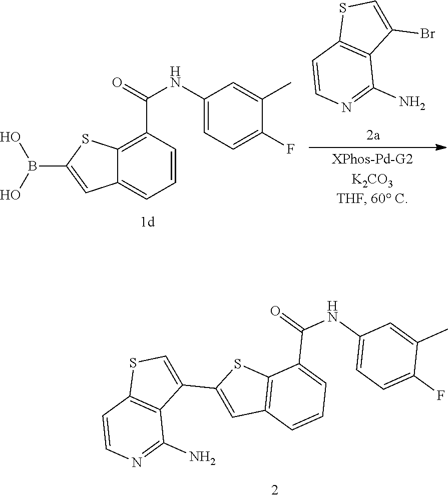

To a reaction flask containing THF (1.0 mL) was sequentially added compound 1d (50.0 mg, 0.152 mmol), compound 2a (41.7 mg, 0.182 mmol), potassium carbonate (63.0 mg, 0.456 mmol) and XPhos-Pd-G2 (CAS No.: 1310584-14-5) (11.9 mg, 0.0152 mmol). The resulting mixture was degassed with N₂, then heated to 60° C. and stirred for 3 h. The reaction mixture was concentrated, and then saturated aq. Na₂CO₃ (5.0 mL) was added, followed by extraction with a mixed solvent of CH₂Cl₂/MeOH (v/v=10:1, 5.0 mL*3). The combined organic phases were washed with saturated brine (10.0 mL), dried over anhydrous Na₂SO₄, filtered, and concentrated. The resulting residue was purified on an ISCO chromatography (mobile phase: CH₃CN/H₂O) to afford compound 2 as a pale yellow solid (17.3 mg, yield: 21%). ESI-MS (m/z): 434.9 [M+H]⁺. ¹H NMR (400 MHz, DMSO-d₆) δ 10.53 (s, 1H), 8.32 (d, J=7.6 Hz, 1H), 8.19 (d, J=7.9 Hz, 1H), 8.14 (s, 1H), 7.93 (d, J=6.7 Hz, 1H), 7.76 (d, J=7.1 Hz, 1H), 7.71 (m, 2H), 7.68 (s, 1H), 7.66-7.61 (m, 1H), 7.31 (s, 2H), 7.19 (m, 1H), 2.27 (s, 3H).

### Example 3

### N-(4-fluoro-3-methylphenyl)-2-(1H-indazol-7-yl)benzo[b]thiophene-7-carboxamide (compound 3)

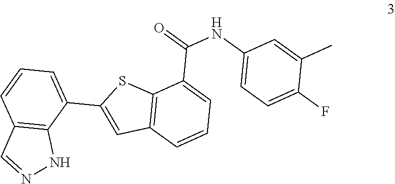 [SMILES: Cc1cc(NC(=O)c2cccc3cc(-c4cccc5cn[nH]c45)sc23)ccc1F]

Compound 3 was prepared by the following step:

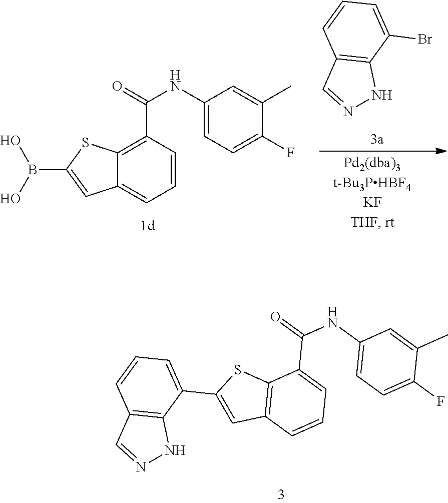

To a reaction flask containing THF (1.0 mL) was sequentially added compound 1d (50.0 mg, 0.152 mmol) compound 3a (35.8 mg, 0.182 mmol), potassium fluoride (29.0 mg, 0.502 mmol), Pd₂(dba)₃ (13.7 mg, 0.0152 mmol) and t-Bu₃P·HBF₄ (10.4 mg, 0.0360 mmol). The resulting mixture was degassed with N₂ and stirred at room temperature for 3 h. The reaction mixture was concentrated, and then saturated aq. Na₂CO₃ (5.0 mL) was added, followed by extraction with a mixed solvent of CH₂Cl₂/MeOH (v/v=10:1, 5.0 mL*3). The combined organic phases were washed with saturated brine (10.0 mL), dried over anhydrous Na₂SO₄, filtered, and concentrated.

The resulting residue was purified on an ISCO chromatography (mobile phase: CH₃CN/H₂O) to afford compound 3 as a pale yellow solid (10.0 mg, yield: 16%). ESI-MS (m/z): 402.0 [M+H]⁺. ¹H NMR (400 MHz, DMSO-d₆) δ 13.43 (s, 1H), 10.50 (s, 1H), 8.29-8.20 (m, 2H), 8.13 (d, J=8.5 Hz, 2H), 7.86 (d, J=8.1 Hz, 1H), 7.80 (d, J=7.0 Hz, 1H), 7.72-7.60 (m, 3H), 7.31-7.23 (m, 1H), 7.22-7.14 (m, 1H), 2.28 (s, 3H).

### Example 4

The synthetic procedures for compounds 4aa-4 bp refer to the synthesis of Compound 1 in Example 1, Compound 2 in Example 2, or Compound 3 in Example 3. The structures of compounds 4aa-4 bp are shown in Table 1.

TABLE 1

| | | MS | |
|---|---|---|---|
| Cpds | Structures | [M + 1]⁺ | ¹H NMR |
| 4aa | 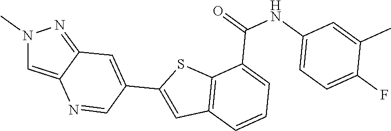 | 417.0 | ¹H NMR (400 MHz, DMSO-d₆) δ 10.50 (s, 1H), 9.04 (d, J = 1.6 Hz, 1H), 8.72 (s, 1H), 8.42- 8.36 (m, 1H), 8.22 (d, J = 7.6 Hz, 1H), 8.16-8.08 (m, 2H), 7.80 (dd, J = 7.0, 2.1 Hz, 1H), 7.67- 7.58 (m, 2H), 7.21-7.13 (m, 1H), 4.25 (s, 3H), 2.28 (s, 3H). |
| 4ab | 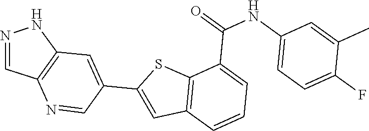 [SMILES: Cc1cc(NC(=O)c2cccc3cc(-c4cnc5cn[nH]c5c4)sc23)ccc1F] | 403.0 | ¹H NMR (400 MHz, DMSO-d₆) δ 10.51 (s, 1H), 9.05 (s, 1H), 8.36 (s, 1H), 8.32 (s, 1H), 8.23 (d, J = 7.5 Hz, 1H), 8.17 (s, 1H), 8.14 (d, J = 7.7 Hz, 1H), 7.79 (d, J = 7.2 Hz, 1H), 7.67-7.59 (m, 2H), 7.23-7.13 (m, 1H), 2.28 (s, 3H). |
| 4ac | 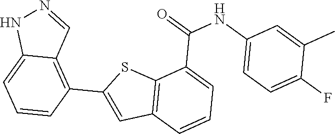 [SMILES: Cc1cc(NC(=O)c2cccc3cc(-c4cccc5[nH]ncc45)sc23)ccc1F] | 402.0 | ¹H NMR (400 MHz, DMSO-d₆) δ 10.50 (s, 1H), 8.56 (s, 1H), 8.22 (d, J = 7.5 Hz, 1H), 8.18 (d, J = 7.9 Hz, 1H), 8.14 (s, 1H), 7.84-7.77 (m, 1H), 7.68-7.60 (m, 3H), 7.53 (d, J = 7.2 Hz, 1H), 7.48 (d, J = 8.0 Hz, 1H), 7.21-7.14 (m, 1H), 2.29 (s, 3H). |
| 4ad | 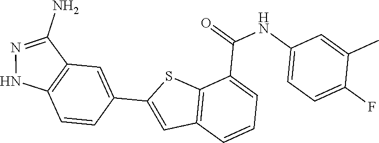 [SMILES: Cc1cc(NC(=O)c2cccc3cc(-c4ccc5[nH]nc(N)c5c4)sc23)ccc1F] | 417.0 | ¹H NMR (400 MHz, DMSO-d₆) δ 10.45 (s, 1H), 8.33 (s, 1H), 8.14 (d, J = 7.4 Hz, 1H), 8.05 (d, J= 7.8 Hz, 1H), 7.89 (d, J = 8.8 Hz, 1H), 7.84-7.76 (m, 2H), 7.69-7.61 (m, 1H), 7.60-7.52 (m, 1H), 7.42 (d, J = 8.8 Hz, 1H), 7.21-7.13 (m, 1H), 2.28 (s, 3H). |
| 4ae | 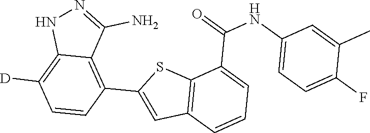 [SMILES: [2H]c1ccc(-c2cc3cccc(C(=O)Nc4ccc(F)c(C)c4)c3s2)c2c(N)n[nH]c12] | 435.1 | |
| 4af | 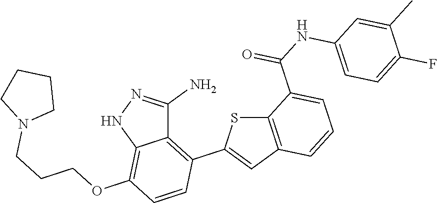 [SMILES: Cc1cc(NC(=O)c2cccc3cc(-c4ccc(OCCCN5CCCC5)c5[nH]nc(N)c45)sc23)ccc1F] | 544.2 | |
| 4ag | 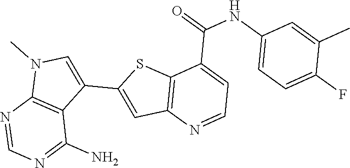 [SMILES: Cc1cc(NC(=O)c2ccnc3cc(-c4cn(C)c5ncnc(N)c45)sc23)ccc1F] | 433.1 | ¹H NMR (400 MHz, DMSO-d₆) δ 10.72 (s, 1H), 8.87 (d, J = 4.9 Hz, 1H), 8.22 (s, 1H), 8.06 (d, J = 5.1 Hz, 1H), 7.81-7.75 (m, 1H), 7.71 (s, 1H), 7.67-7.60 (m, 1H), 7.54 (s, 1H), 7.26- 7.12 (m, 1H), 6.60 (s, 2H), 3.78 (s, 3H), 2.28 (d, J = 2.0 Hz, 3H). |
| 4ah | 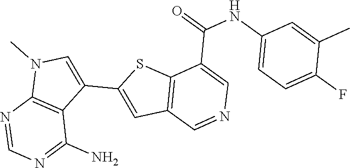 [SMILES: Cc1cc(NC(=O)c2cncc3cc(-c4cn(C)c5ncnc(N)c45)sc23)ccc1F] | 433.1 | ¹H NMR (400 MHz, DMSO-d₆) δ 10.63 (s, 1H), 9.26 (s, 1H), 9.21 (s, 1H), 8.22 (s, 1H), 7.78 (dd, J = 6.9, 2.5 Hz, 1H), 7.67- 7.62 (m, 2H), 7.58 (s, 1H), 7.18 (t, J = 9.2 Hz, 1H), 6.57 (s, 2H), 3.78 (s, 3H), 2.29 (s, 3H). |
| 4ai | 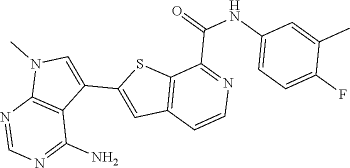 [SMILES: Cc1cc(NC(=O)c2nccc3cc(-c4cn(C)c5ncnc(N)c45)sc23)ccc1F] | 433.1 | ¹H NMR (400 MHz, DMSO-d₆) δ 10.92 (s, 1H), 8.65 (d, J = 5.3 Hz, 1H), 8.23 (s, 1H), 8.09 (d, J = 5.3 Hz, 1H), 7.96 (dd, J = 7.2, 2.7 Hz, 1H), 7.83-7.73 (m, 2H), 7.55 (s, 1H), 7.22-7.12 (m, 1H), 6.61 (br s, 2H), 3.79 (s, 3H), 2.28 (s, 3H). |
| 4aj | 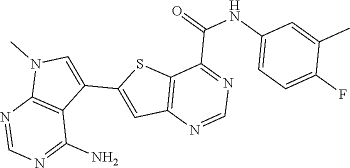 [SMILES: Cc1cc(NC(=O)c2ncnc3cc(-c4cn(C)c5ncnc(N)c45)sc23)ccc1F] | 434.1 | |
| 4ak | 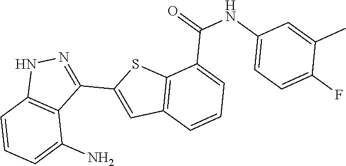 [SMILES: Cc1cc(NC(=O)c2cccc3cc(-c4n[nH]c5cccc(N)c45)sc23)ccc1F] | 417.0 | ¹H NMR (400 MHz, DMSO-d₆) 13.19 (s, 1H), 10.46 (s, 1H), 8.20 (d, J = 7.6 Hz, 1H), 8.16 (d, J = 7.8 Hz, 1H), 7.85 (s, 1H), 7.81-7.74 (m, 2H), 7.68-7.55 (m, 2H), 7.22-7.09 (m, 2H), 6.81 (d, J = 8.2 Hz, 1H), 6.41 (d, J = 7.3 Hz, 1H), 2.28 (s, 3H). |
| 4al | 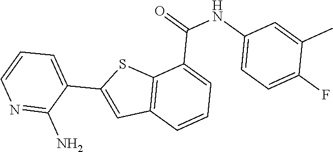 [SMILES: Cc1cc(NC(=O)c2cccc3cc(-c4cccnc4N)sc23)ccc1F] | 378.5 | ¹H NMR (400 MHz, DMSO-d₆) δ 10.51 (s, 1H), 8.28 (d, J = 7.6 Hz, 1H), 8.16 (d, J = 7.8 Hz, 1H), 8.06 (d, J = 5.8 Hz, 1H), 7.99 (d, J = 7.4 Hz, 1H), 7.79- 7.74 (m, 2H), 7.68-7.60 (m, 2H), 7.47 (s, 1H), 7.17 (t, J = 9.2 Hz, 1H), 6.98-6.93 (m, 1H), 2.27 (s, 3H). |
| 4am | 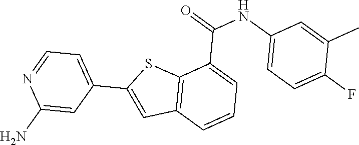 [SMILES: Cc1cc(NC(=O)c2cccc3cc(-c4ccnc(N)c4)sc23)ccc1F] | 378.1 | ¹H NMR (400 MHz, DMSO-d₆) δ 10.55 (s, 1H), 8.38-8.31 (m, 2H), 8.21 (d, J = 7.8 Hz, 1H), 8.03 (d, J = 6.7 Hz, 1H), 7.86 (s, 1H), 7.81-7.76 (m, 1H), 7.72- 7.66 (m, 1H), 7.66-7.61 (m, 1H), 7.36 (d, J = 6.8 Hz, 1H), 7.28 (s, 1H), 7.21-7.14 (m, 1H), 2.28 (d, J = 2.0 Hz, 3H). |
| 4an | 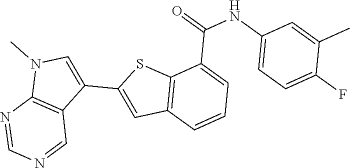 [SMILES: Cc1cc(NC(=O)c2cccc3cc(-c4cn(C)c5ncncc45)sc23)ccc1F] | 417.1 | ¹H NMR (400 MHz, DMSO-d₆) δ 10.47 (s, 1H), 9.56 (s, 1H), 8.98 (s, 1H), 8.27 (s, 1H), 8.17 (d, J = 7.6 Hz, 1H), 8.06 (d, J = 7.7 Hz, 1H), 7.98 (s, 1H), 7.83- 7.75 (m, 1H), 7.68-7.55 (m, 2H), 7.21-7.11 (m, 1H), 3.91 (s, 3H), 2.28 (s, 3H). |
| 4ao | 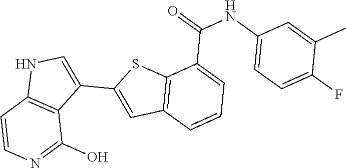 [SMILES: Cc1cc(NC(=O)c2cccc3cc(-c4c[nH]c5ccnc(O)c45)sc23)ccc1F] | 418.1 | ¹H NMR (400 MHz, DMSO-d₆) δ 11.79 (s, 1H), 10.89-10.81 (m, 1H), 10.41 (s, 1H), 8.59 (s, 1H), 8.06 (d, J = 7.4 Hz, 1H), 7.97 (d, J = 7.7 Hz, 1H), 7.80 (dd, J = 7.0, 2.6 Hz, 1H), 7.66- 7.60 (m, 1H), 7.59 (s, 1H), 7.54- 7.46 (m, 1H), 7.21-7.12 (m, 1H), 7.10-7.02 (m, 1H), 6.41 (d, J = 6.8 Hz, 1H), 2.28 (s, 3H). |
| 4ap | 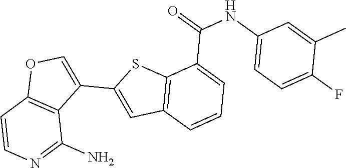 [SMILES: Cc1cc(NC(=O)c2cccc3cc(-c4coc5ccnc(N)c45)sc23)ccc1F] | 418.0 | ¹H NMR (400 MHz, DMSO-d₆) δ 10.54 (s, 1H), 8.58 (s, 1H), 8.31 (d, J = 7.4 Hz, 1H), 8.18 (d, J = 7.8 Hz, 1H), 8.05 (d, J = 6.9 Hz, 1H), 7.80-7.75 (m, 1H), 7.71 (s, 1H), 7.69-7.56 (3, 3H), 7.42 (d, J = 6.9 Hz, 1H), 7.20- 7.13 (m, 1H), 2.27 (s, 3H). |
| 4aq | 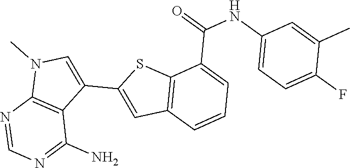 [SMILES: Cc1cc(NC(=O)c2cccc3cc(-c4cn(C)c5ncnc(N)c45)sc23)ccc1F] | 432.1 | ¹H NMR (400 MHz, DMSO-d₆) δ 10.46 (s, 1H), 8.22-8.08 (m, 3H), 7.78 (dd, J = 7.3, 2.6 Hz, 1H), 7.67-7.56 (m, 3H), 7.48 (s, 1H), 7.20-7.14 (m, 1H), 6.51 (s, 2H), 3.78 (s, 3H), 2.28 (d, J = 2.0 Hz, 3H). |
| 4ar | 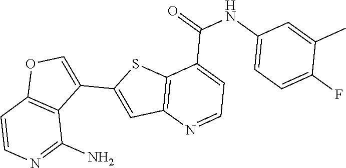 [SMILES: Cc1cc(NC(=O)c2ccnc3cc(-c4coc5ccnc(N)c45)sc23)ccc1F] | 419.0 | ¹H NMR (400 MHz, DMSO-d₆) 10.78 (s, 1H), 8.95 (d, J = 4.9 Hz, 1H), 8.35 (s, 1H), 8.17 (d, J = 5.0 Hz, 1H), 7.95 (d, J = 5.8 Hz, 2H), 7.82-7.77 (m, 1H), 7.83-7.76 (m, 1H), 7.68-7.62 (m, 1H), 7.25-7.15 (m, 1H), 7.01 (d, J = 5.9 Hz, 1H), 5.98 (s, 2H), 2.29 (d, J = 2.0 Hz, 3H). |
| 4as | 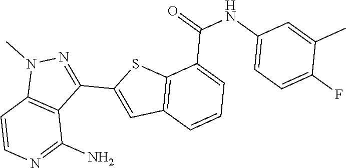 [SMILES: Cc1cc(NC(=O)c2cccc3cc(-c4nn(C)c5ccnc(N)c45)sc23)ccc1F] | 432.1 | ¹H NMR (400 MHz, DMSO-d₆) δ 10.49 (s, 1H), 8.26 (d, J = 7.5 Hz, 1H), 8.19 (d, J = 7.8 Hz, 1H), 7.88 (s, 1H), 7.83 (d, J = 6.3 Hz, 1H), 7.77 (dd, J = 7.1, 2.7 Hz, 1H), 7.68-7.59 (m, 2H), 7.22-7.13 (m, 1H), 7.01 (d, J = 6.3 Hz, 1H), 6.62 (br s, 2H), 4.05 (s, 3H), 2.28 (s, 3H). |
| 4at | 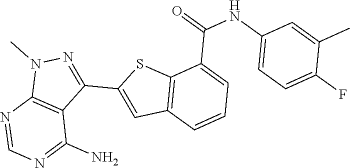 [SMILES: Cc1cc(NC(=O)c2cccc3cc(-c4nn(C)c5ncnc(N)c45)sc23)ccc1F] | 433.1 | ¹H NMR (400 MHz, DMSO-d₆) δ 10.48 (s, 1H), 8.30 (s, 1H), 8.28-8.21 (m, 1H), 8.18 (d, J = 7.7 Hz, 1H), 7.85 (s, 1H), 7.78 (dd, J = 7.4, 2.6 Hz, 1H), 7.63 (m, 2H), 7.27 (s, 2H), 7.17 (t, J = 9.2 Hz, 1H), 3.98 (s, 3H), 2.28 (d, J = 2.0 Hz, 3H). |
| 4au | 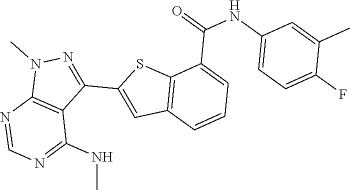 [SMILES: CNc1ncnc2c1c(-c1cc3cccc(C(=O)Nc4ccc(F)c(C)c4)c3s1)nn2C] | 447.1 | |
| 4av | 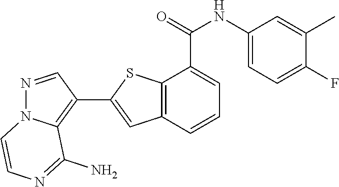 [SMILES: Cc1cc(NC(=O)c2cccc3cc(-c4cnn5ccnc(N)c45)sc23)ccc1F] | 418.1 | |
| 4aw | 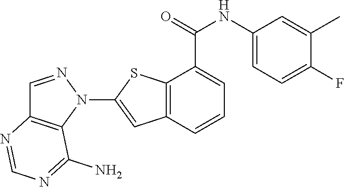 [SMILES: Cc1cc(NC(=O)c2cccc3cc(-n4ncc5ncnc(N)c54)sc23)ccc1F] | 419.1 | |
| 4ax | 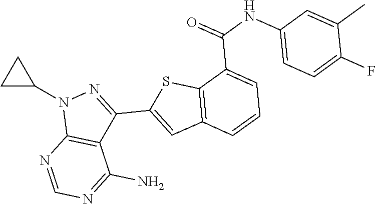 [SMILES: Cc1cc(NC(=O)c2cccc3cc(-c4nn(C5CC5)c5ncnc(N)c45)sc23)ccc1F] | 459.1 | |
| 4ay | 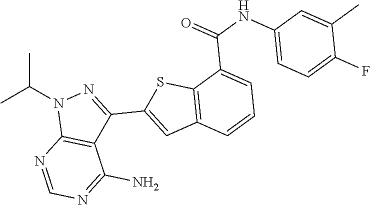 [SMILES: Cc1cc(NC(=O)c2cccc3cc(-c4nn(C(C)C)c5ncnc(N)c45)sc23)ccc1F] | 461.2 | |
| 4az | 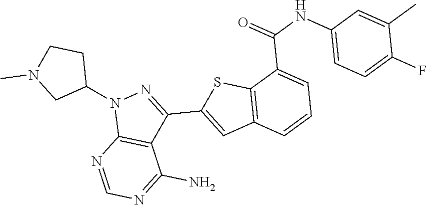 [SMILES: Cc1cc(NC(=O)c2cccc3cc(-c4nn(C5CCN(C)C5)c5ncnc(N)c45)sc23)ccc1F] | 502.2 | |
| 4ba | 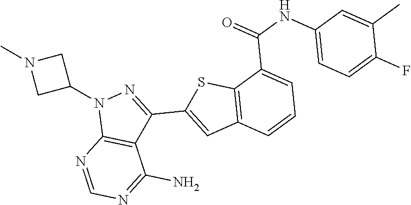 [SMILES: Cc1cc(NC(=O)c2cccc3cc(-c4nn(C5CN(C)C5)c5ncnc(N)c45)sc23)ccc1F] | 488.2 | |
| 4bb | 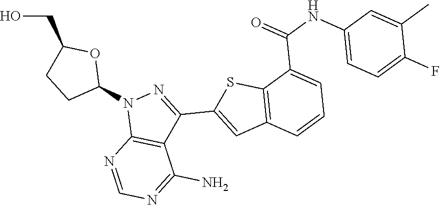 [SMILES: Cc1cc(NC(=O)c2cccc3cc(-c4nn([C@H]5CC[C@@H](CO)O5)c5ncnc(N)c45)sc23)ccc1F] | 519.2 | |
| 4bc | 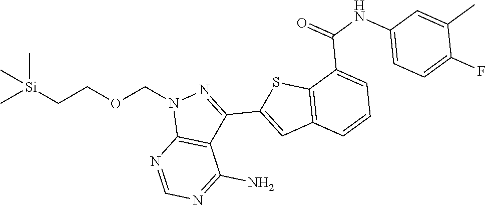 [SMILES: Cc1cc(NC(=O)c2cccc3cc(-c4nn(COCC[Si](C)(C)C)c5ncnc(N)c45)sc23)ccc1F] | 549.2 | |
| 4bd | 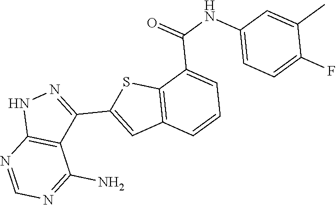 [SMILES: Cc1cc(NC(=O)c2cccc3cc(-c4n[nH]c5ncnc(N)c45)sc23)ccc1F] | 419.1 | |
| 4be | 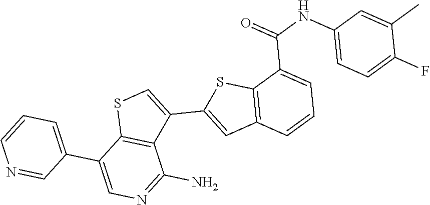 [SMILES: Cc1cc(NC(=O)c2cccc3cc(-c4csc5c(-c6cccnc6)cnc(N)c45)sc23)ccc1F] | 511.1 | |
| 4bf | 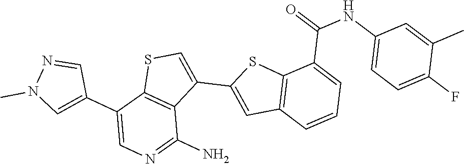 | 514.1 | |
| 4bg | 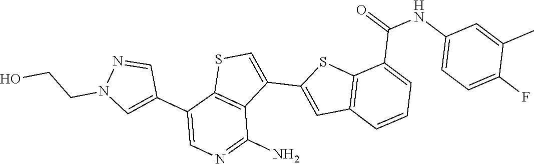 [SMILES: Cc1cc(NC(=O)c2cccc3cc(-c4csc5c(-c6cnn(CCO)c6)cnc(N)c45)sc23)ccc1F] | 544.1 | |
| 4bh | 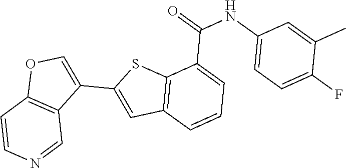 [SMILES: Cc1cc(NC(=O)c2cccc3cc(-c4coc5ccncc45)sc23)ccc1F] | 403.4 | ¹H NMR (400 MHz, DMSO-d₆) 10.50 (s, 1H), 9.49 (s, 1H), 8.75 (s, 1H), 8.62 (d, J = 5.6 Hz, 1H), 8.25 (d, J = 7.5 Hz, 1H), 8.20-8.06 (m, 2H), 7.86-7.73 (m, 2H), 7.68-7.58 (m, 2H), 7.23-7.11 (m, 1H), 2.28 (s, 3H). |
| 4bi | 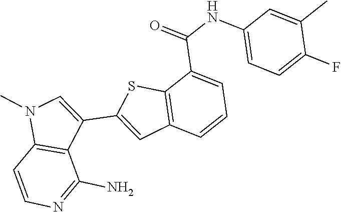 [SMILES: Cc1cc(NC(=O)c2cccc3cc(-c4cn(C)c5ccnc(N)c45)sc23)ccc1F] | 431.1 | |
| 4bj | 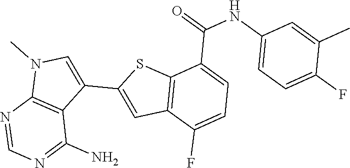 [SMILES: Cc1cc(NC(=O)c2ccc(F)c3cc(-c4cn(C)c5ncnc(N)c45)sc23)ccc1F] | 450.1 | ¹H NMR (600 MHz, DMSO-d₆) δ 10.46 (s, 1H), 8.28-8.23 (m, 1H), 8.21 (s, 1H), 7.79-7.73 (m, 1H), 7.64 (s, 1H), 7.63- 7.59 (m, 1H), 7.47-7.42 (m, 2H), 7.16 (t, J = 9.2 Hz, 1H), 6.53 (s, 2H), 3.77 (s, 3H), 2.27 (s, 3H). |
| 4bk | 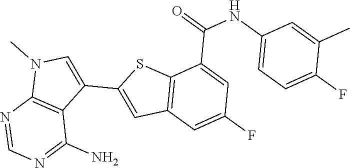 [SMILES: Cc1cc(NC(=O)c2cc(F)cc3cc(-c4cn(C)c5ncnc(N)c45)sc23)ccc1F] | 450.1 | |
| 4b1 | 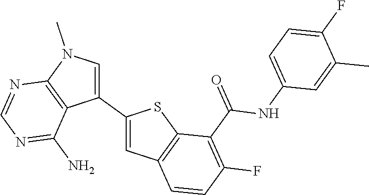 [SMILES: Cc1cc(NC(=O)c2c(F)ccc3cc(-c4cn(C)c5ncnc(N)c45)sc23)ccc1F] | 450.1 | |
| 4bm | 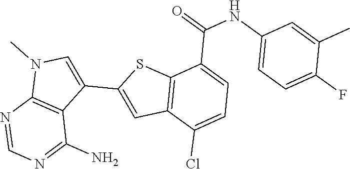 [SMILES: Cc1cc(NC(=O)c2ccc(Cl)c3cc(-c4cn(C)c5ncnc(N)c45)sc23)ccc1F] | 466.1 | |
| 4bn | 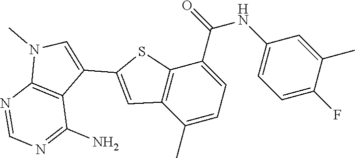 [SMILES: Cc1cc(NC(=O)c2ccc(C)c3cc(-c4cn(C)c5ncnc(N)c45)sc23)ccc1F] | 446.1 | |
| 4bo | 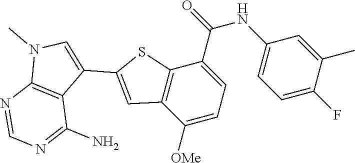 [SMILES: COc1ccc(C(=O)Nc2ccc(F)c(C)c2)c2sc(-c3cn(C)c4ncnc(N)c34)cc12] | 462.1 | |
| 4bp | 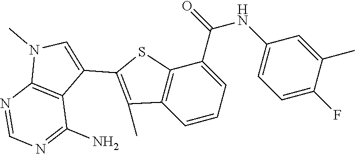 [SMILES: Cc1cc(NC(=O)c2cccc3c(C)c(-c4cn(C)c5ncnc(N)c45)sc23)ccc1F] | 446.1 | ¹H NMR (400 MHz, DMSO-d₆) δ 10.46 (s, 1H), 8.23 (d, J = 7.4 Hz, 1H), 8.19 (s, 1H), 8.04 (d, J = 7.7 Hz, 1H), 7.79-7.73 (m, 1H), 7.69-7.60 (m, 2H), 7.43 (s, 1H), 7.16 (t, J = 9.2 Hz, 1H), 6.21 (s, 2H), 3.79 (s, 3H), 2.37 (s, 3H), 2.27 (d, J = 1.4 Hz, 3H). |

### Example 5

### 2-(4-amino-7-methyl-711-pyrrolo[2,3-d]pyrimidin-5-yl)-N-(3-((4-methylpiperazin-1-yl)methyl)-5-(trifluoromethyl)phenyl)benzo[b]thiophene-7-carboxamide (compound 5)

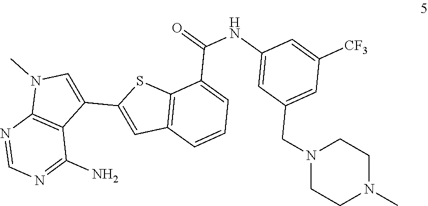 [SMILES: CN1CCN(Cc2cc(NC(=O)c3cccc4cc(-c5cn(C)c6ncnc(N)c56)sc34)cc(C(F)(F)F)c2)CC1]

The intermediate 5c was prepared by the following steps:

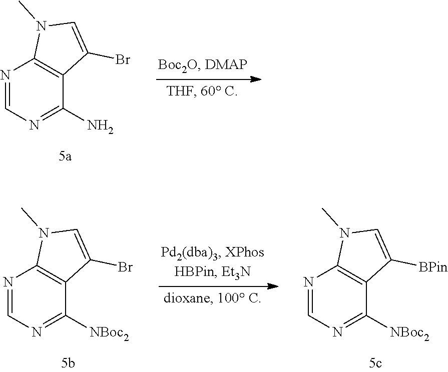

Step 1: To a solution of compound 5a (10.0 g, 44.0 mmol) and 4-dimethylaminopyridine (538.0 mg, 4.40 mmol) in THF (100.0 mL) was added di-tert-butyl dicarbonate (28.9 g, 132.1 mmol, 30.0 mL). The resultant mixture was stirred at 60° C. until TLC showed the reaction was completed. After cooling to room temperature, the reaction mixture was added EtOAc (100.0 mL) and filtered. The filter cake was washed with EtOAc (30.0 mL) and dried to afford compound 5b (18.00 g, yield: 96%). ¹H NMR (400 MHz, DMSO-d₆) δ 8.80 (s, 1H), 7.93 (s, 1H), 3.85 (s, 3H), 1.34 (s, 18H).

Step 2: To a solution of compound 5b (5.00 g, 11.80 mmol) and XPhos (563.0 mg, 1.18 mmol) in dioxane (50.0 mL) was added triethylamine (4.75 g, 46.9 mmol, 6.5 mL) and Pd₂(dba)₃ (1.08 g, 1.18 mmol). The resultant mixture was degassed with N₂ for three times, followed by slowly addition of HBpin (CAS No.: 25015-63-8) (6.00 g, 46.9 mmol, 6.8 mL). The reaction mixture was stirred at 100° C. until TLC showed the reaction was completed. The mixture was concentrated to dryness, then purified on an ISCO chromatography (mobile phase: petroleum ether/ethyl acetate) to afford compound 5c as a white powder (4.10 g, yield: 73%). ESI-MS (m/z): 475.2 [M+H]⁺. ¹H NMR (400 MHz, DMSO-d₆) δ 8.76 (s, 1H), 8.02 (s, 1H), 3.85 (s, 3H), 1.25 (s, 12H), 1.22 (s, 18H).

Compound 5 was prepared by the following steps:

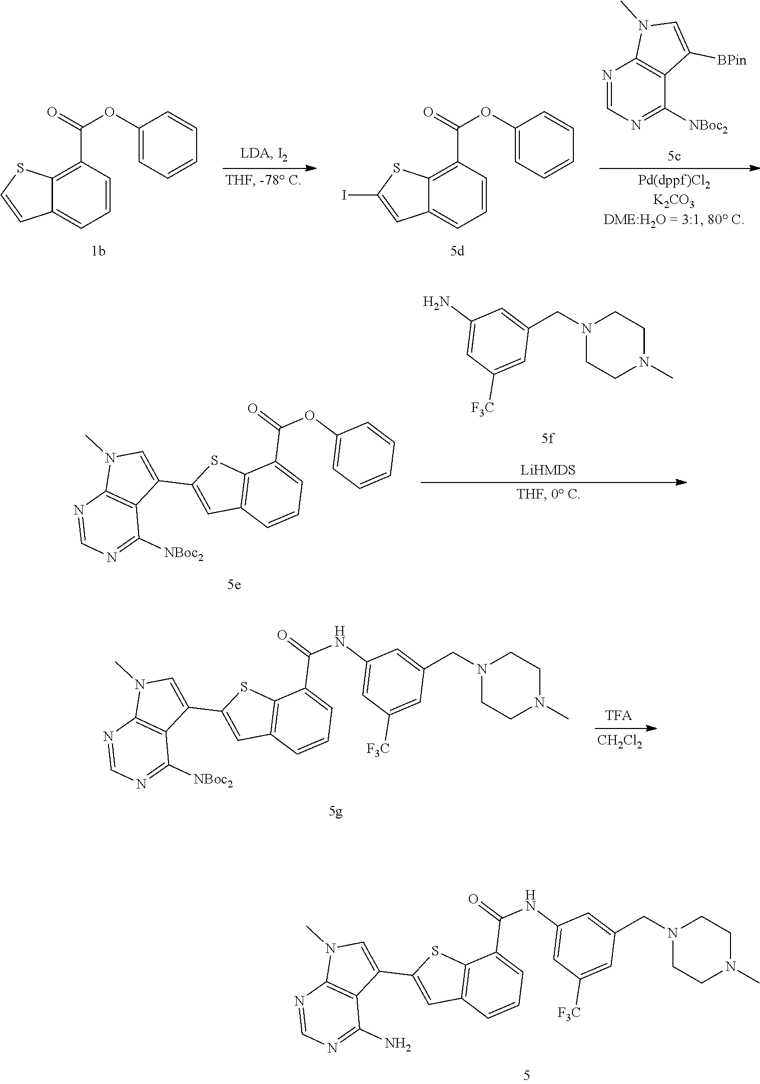

Step 1: To a solution of compound 1b (2.00 g, 8.00 mmol) in dry THF (15.0 mL) was slowly added LDA (6.0 mL, 2.0 M in THF, 12.00 mmol) dropwise at −78° C., followed by addition of a solution of iodine (3.10 g, 12.00 mmol) in dry THF (5.0 mL) after stirring for 1 h. The resultant mixture was stirred for 30 min and then warmed to room temperature. The reaction mixture was quenched with saturated aq. Na₂CO₃ (50.0 mL) and Na₂S₂O₃ (50.0 mL), then extracted with ethyl acetate (100.0 mL*3). The combined organic phases were washed with saturated brine, dried over anhydrous Na₂SO₄, and concentrated. The resulting residue was purified on an ISCO chromatography (mobile phase: petroleum ether/ethyl acetate) to afford compound 5d (1.62 g, yield: 53%). ¹H NMR (400 MHz, DMSO-d₆) δ 8.25 (dd, J=7.5, 0.8 Hz, 1H), 8.20 (d, J=7.9 Hz, 1H), 7.92 (s, 1H), 7.64-7.58 (m, 1H), 7.55-7.48 (m, 2H), 7.40-7.33 (m, 3H).

Step 2: To a reaction flask containing a mixed solvent of DME/H₂O (v/v, 3:1) (30.0 mL) was sequentially added compound 5d (1.20 g, 3.16 mmol), compound 5c (1.65 g, 3.47 mmol), potassium carbonate (1.31 g, 9.47 mmol) and Pd(dppf)Cl₂ CH₂Cl₂ (231.0 mg, 0.316 mmol). The resultant mixture was degassed with N₂ and stirred at 80° C. for 3 h. After the reaction mixture was concentrated, a saturated aq. Na₂CO₃ (50.0 mL) was added to the reaction residue followed by extraction with a mixed solvent of CH₂Cl₂/MeOH (v/v=10:1, 50.0 mL*3). The combined organic phases were washed with saturated brine (100.0 mL), dried over anhydrous Na₂SO₄, filtered, and concentrated. The resulting residue was purified on an ISCO chromatography (mobile phase: CH₂Cl₂/MeOH) to afford compound 5e as a pale yellow solid (1.80 g, yield: 86%). ESI-MS (m/z): 601.2 [M+H]⁺.

Step 3: To a stirred solution of compound 5e (100.0 mg, 0.166 mmol) and compound 5f (55.0 mg, 0.199 mmol) in THF (2.0 mL) was added LiHMDS (0.5 mL, 1 M in THF, 0.50 mmol) at 0° C. The reaction mixture was stirred at 0° C. until TLC showed the reaction was completed. The reaction mixture was quenched with saturated aq. Na₂CO₃ (10.0 mL), followed by extraction with a mixed solvent of CH₂Cl₂/MeOH (v/v=10:1, 10.0 mL*3). The combined organic phases were washed with saturated saturated brine (10.0 mL), dried over anhydrous Na₂SO₄, filtered, and concentrated to afford crude product 5g, which was used directly in Step 4. ESI-MS (m/z): 780.8 [M+H]⁺.

Step 4: To a solution of compound 5g in CH₂Cl₂ (2.0 mL) was added TFA (2.0 mL). The reaction mixture was stirred at room temperature until TLC showed the reaction was completed. After concentration, the reaction mixture was quenched with saturated aq. Na₂CO₃ (20.0 mL), followed by extraction with a mixed solvent of CH₂Cl₂/MeOH (v/v=10:1, 20.0 mL*3). The combined organic phases were washed with saturated brine (50.0 mL), dried over anhydrous Na₂SO₄, filtered, and concentrated. The resulting residue was purified on an ISCO chromatography (mobile phase: CH₃CN/H₂O) to afford compound 5 as a pale yellow solid (35.0 mg, yield: 36% for two steps). ESI-MS (m/z): 580.2 [M+H]⁺. ¹H NMR (400 MHz, DMSO-d₆) δ 10.77 (s, 1H), 8.27-8.21 (m, 2H), 8.20 (s, 1H), 8.10 (d, J=7.8 Hz, 1H), 8.05 (s, 1H), 7.63-7.55 (m, 2H), 7.48 (s, 1H), 7.37 (s, 1H), 6.51 (s, 2H), 3.77 (s, 3H), 3.56 (s, 2H), 2.45-2.23 (m, 8H), 2.15 (s, 3H).

### Example 6

The synthetic procedure for compounds 6aa-6bg refers to the synthesis of Compound 5 in Example 5. The structures of compounds 6aa-6bg are shown in Table 2.

TABLE 2

| | | MS | |
|---|---|---|---|
| Cpds | Structures | [M + 1]⁺ | ¹H NMR |
| 6aa | 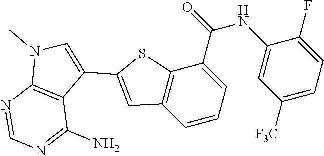 [SMILES: Cn1cc(-c2cc3cccc(C(=O)Nc4cc(C(F)(F)F)ccc4F)c3s2)c2c(N)ncnc21] | 486.1 | ¹H NMR (400 MHz, DMSO-d₆) δ 10.64 (s, 1H), 8.27-8.18 (m, 2H), 8.17-8.08 (m, 2H), 7.77-7.70 (m, 1H), 7.66-7.58 (m, 3H), 7.50 (s, 1H), 6.51 (s, 2H), 3.77 (s, 3H). |
| 6ab | 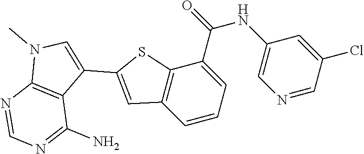 [SMILES: Cn1cc(-c2cc3cccc(C(=O)Nc4cncc(Cl)c4)c3s2)c2c(N)ncnc21] | 435.1 | ¹H NMR (400 MHz, DMSO-d₆) δ 10.87 (s, 1H), 8.94 (d, J = 2.2 Hz, 1H), 8.46 (t, J = 2.2 Hz, 1H), 8.42 (d, J = 2.3 Hz, 1H), 8.21 (d, J = 7.8 Hz, 1H), 8.14 (d, J = 7.8 Hz, 1H), 7.67-7.58 (m, 2H), 7.50 (s, 1H), 6.51 (s, 2H), 3.77 (s, 3H). |
| 6ac | 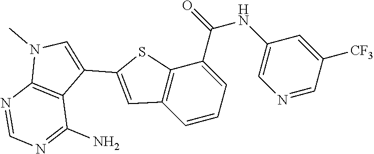 [SMILES: Cn1cc(-c2cc3cccc(C(=O)Nc4cncc(C(F)(F)F)c4)c3s2)c2c(N)ncnc21] | 469.1 | ¹H NMR (400 MHz, DMSO-d₆) δ 11.02 (s, 1H), 9.27 (s, 1H), 8.76 (s, 1H), 8.72 (s, 1H), 8.27 (s, 1H), 8.25- 8.20 (m, 1H), 8.16 (d, J = 7.7 Hz, 1H), 7.67-7.62 (m, 2H), 7.51 (s, 1H), 6.53 (s, 2H), 3.78 (s, 3H). |
| 6ad | 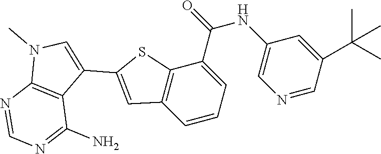 [SMILES: Cn1cc(-c2cc3cccc(C(=O)Nc4cncc(C(C)(C)C)c4)c3s2)c2c(N)ncnc21] | 457.1 | ¹H NMR (400 MHz, DMSO-d₆) δ 10.61 (s, 1H), 8.91 (d, J= 2.2 Hz, 1H), 8.45 (d, J = 2.2 Hz, 1H), 8.27-8.19 (m, 3H), 8.17-8.10 (m, 1H), 7.66- 7.57 (m, 2H), 7.50 (s, 1H), 6.53 (s, 2H), 3.78 (s, 3H), 1.36 (s, 9H). |
| 6ae | 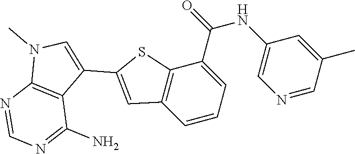 [SMILES: Cc1cncc(NC(=O)c2cccc3cc(-c4cn(C)c5ncnc(N)c45)sc23)c1] | 415.1 | ¹H NMR (400 MHz, DMSO-d₆) δ 10.62 (s, 1H), 8.77 (s, 1H), 8.23-8.18 (m, 3H), 8.15-8.10 (m, 2H), 7.63- 7.58 (m, 2H), 7.49 (s, 1H), 6.51 (s, 2H), 3.77 (s, 3H), 2.35 (s, 3H). |
| 6af | 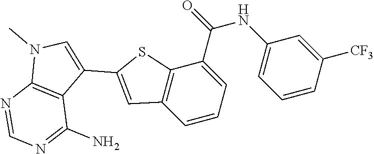 [SMILES: Cn1cc(-c2cc3cccc(C(=O)Nc4cccc(C(F)(F)F)c4)c3s2)c2c(N)ncnc21] | 468.1 | ¹H NMR (400 MHz, DMSO-d₆) δ 10.78 (s, 1H), 8.32 (d, J = 2.0 Hz, 1H), 8.26-8.18 (m, 2H), 8.16-8.09 (m, 2H), 7.69-7.57 (m, 3H), 7.50 (d, J = 8.1 Hz, 2H), 6.51 (s, 2H), 3.77 (s, 3H). |
| 6ag | 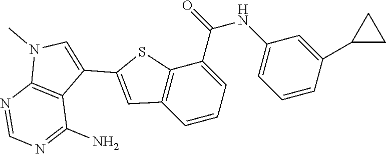 [SMILES: Cn1cc(-c2cc3cccc(C(=O)Nc4cccc(C5CC5)c4)c3s2)c2c(N)ncnc21] | 440.1 | ¹H NMR (400 MHz, DMSO-d₆) δ 10.37 (s, 1H), 8.22-8.15 (m, 2H), 8.09 (d, J = 7.8 Hz, 1H), 7.67-7.53 (m, 4H), 7.47 (s, 1H), 7.25 (t, J = 7.9 Hz, 1H), 6.92-6.81 (m, 1H), 6.50 (s, 2H), 3.77 (s, 3H), 2.02-1.87 (m, 1H), 1.02-0.94 (m, 2H), 0.73-0.63 (m, 2H). |
| 6ah | 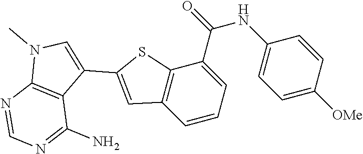 [SMILES: COc1ccc(NC(=O)c2cccc3cc(-c4cn(C)c5ncnc(N)c45)sc23)cc1] | 430.1 | |
| 6ai | 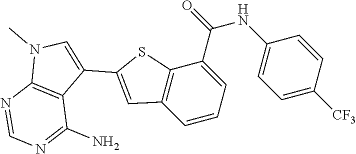 [SMILES: Cn1cc(-c2cc3cccc(C(=O)Nc4ccc(C(F)(F)F)cc4)c3s2)c2c(N)ncnc21] | 468.1 | |
| 6aj | 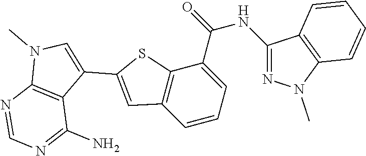 [SMILES: Cn1cc(-c2cc3cccc(C(=O)Nc4nn(C)c5ccccc45)c3s2)c2c(N)ncnc21] | 454.1 | |
| 6ak | 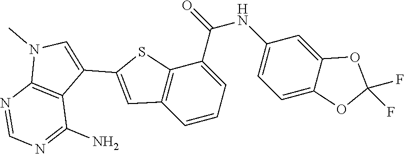 [SMILES: Cn1cc(-c2cc3cccc(C(=O)Nc4ccc5c(c4)OC(F)(F)O5)c3s2)c2c(N)ncnc21] | 480.1 | |
| 6al | 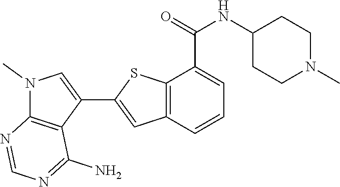 | 421.2 | ¹H NMR (400 MHz, DMSO-d₆) δ 8.53 (d, J = 7.7 Hz, 1H), 8.20 (s, 1H), 8.07-7.97 (m, 2H), 7.56 (s, 1H), 7.53- 7.46 (m, 1H), 7.43 (s, 1H), 6.48 (s, 2H), 3.77 (s, 3H), 2.85-2.76 (m, 2H), 2.18 (s, 3H), 2.04-1.89 (m, 2H), 1.86- 1.75 (m, 2H), 1.70-1.55 (m, 2H). |
| 6am | 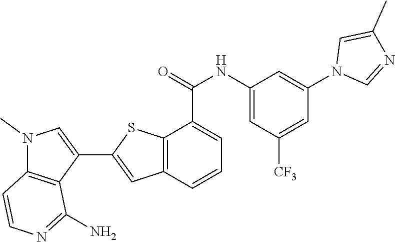 [SMILES: Cc1cn(-c2cc(NC(=O)c3cccc4cc(-c5cn(C)c6ccnc(N)c56)sc34)cc(C(F)(F)F)c2)cn1] | 547.1 | |
| 6an | 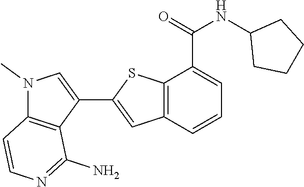 [SMILES: Cn1cc(-c2cc3cccc(C(=O)NC4CCCC4)c3s2)c2c(N)nccc21] | 391.2 | |
| 6ao | 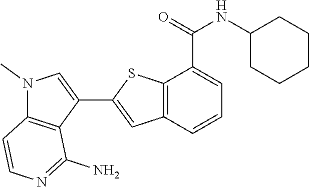 [SMILES: Cn1cc(-c2cc3cccc(C(=O)NC4CCCCC4)c3s2)c2c(N)nccc21] | 405.2 | |
| 6ap | 2 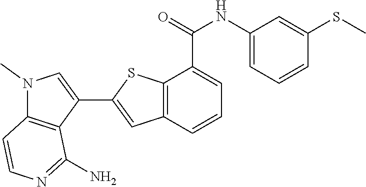 | 445.1 | |
| 6aq | 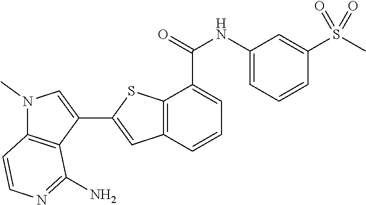 | 477.1 | |
| 6ar | 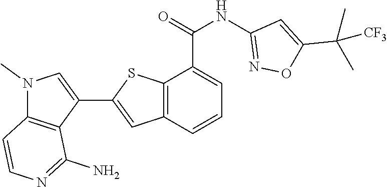 [SMILES: Cn1cc(-c2cc3cccc(C(=O)Nc4cc(C(C)(C)C(F)(F)F)on4)c3s2)c2c(N)nccc21] | 500.1 | |
| 6as | 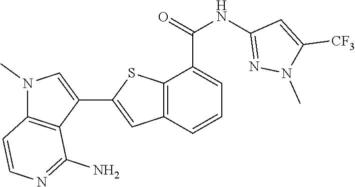 [SMILES: Cn1nc(NC(=O)c2cccc3cc(-c4cn(C)c5ccnc(N)c45)sc23)cc1C(F)(F)F] | 471.1 | |
| 6at | 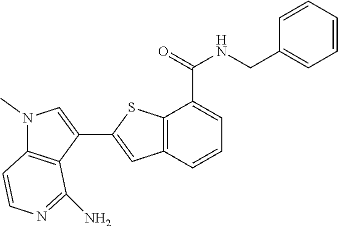 [SMILES: Cn1cc(-c2cc3cccc(C(=O)NCc4ccccc4)c3s2)c2c(N)nccc21] | 413.1 | |
| 6au | 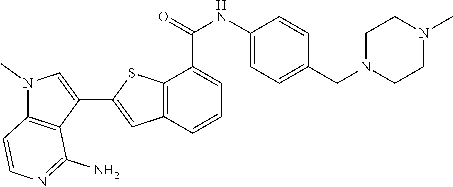 | 511.2 | |
| 6av | 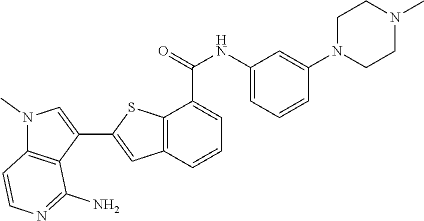 | 497.2 | |
| 6aw | 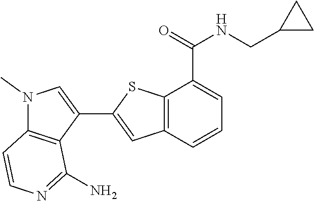 [SMILES: Cn1cc(-c2cc3cccc(C(=O)NCC4CC4)c3s2)c2c(N)nccc21] | 377.1 | |
| 6ax | 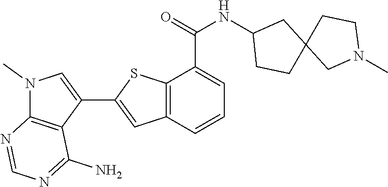 | 460.2 | |
| 6ay | 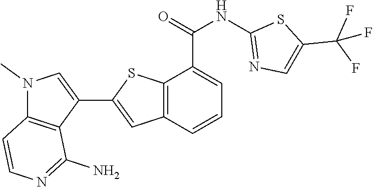 [SMILES: Cn1cc(-c2cc3cccc(C(=O)Nc4ncc(C(F)(F)F)s4)c3s2)c2c(N)nccc21] | 474.1 | |
| 6az | 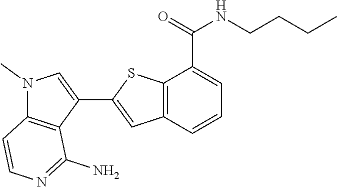 [SMILES: CCCCNC(=O)c1cccc2cc(-c3cn(C)c4ccnc(N)c34)sc12] | 379.2 | |
| 6ba | 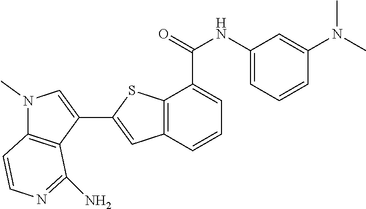 | 442.2 | |
| 6bb | 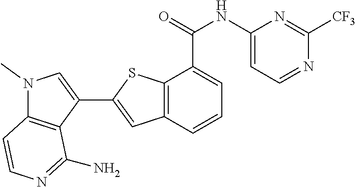 [SMILES: Cn1cc(-c2cc3cccc(C(=O)Nc4ccnc(C(F)(F)F)n4)c3s2)c2c(N)nccc21] | 469.1 | |
| 6bc | 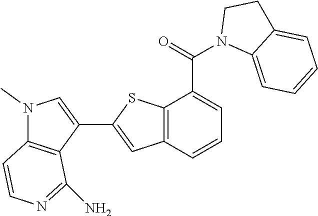 [SMILES: Cn1cc(-c2cc3cccc(C(=O)N4CCc5ccccc54)c3s2)c2c(N)nccc21] | 425.1 | |
| 6bd | 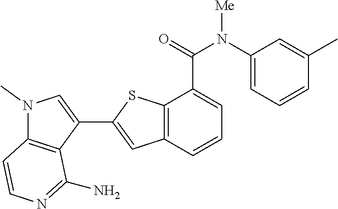 | 427.2 | |
| 6be | 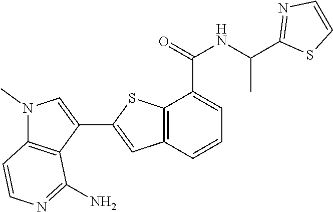 [SMILES: CC(NC(=O)c1cccc2cc(-c3cn(C)c4ccnc(N)c34)sc12)c1nccs1] | 434.1 | |
| 6bf | 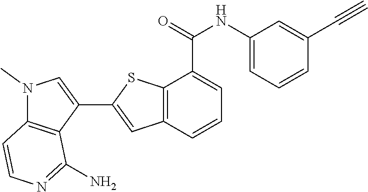 [SMILES: C#Cc1cccc(NC(=O)c2cccc3cc(-c4cn(C)c5ccnc(N)c45)sc23)c1] | 423.1 | |
| 6bg | 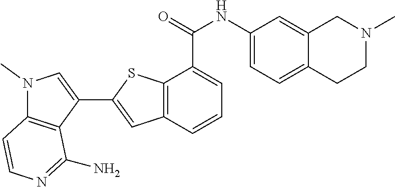 | 468.2 | |

### Example 7

### 2-(4-amino-7-methyl-7H-pyrrolo[2,3-d]pyrimidin-5-yl)-3-bromo-N-(3-(trifluoromethyl)phenyl)benzo[b]thiophene-7-carboxamide (compound 7)

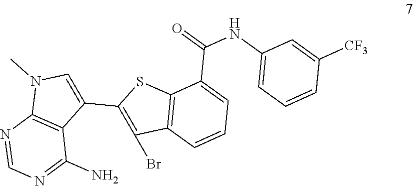 [SMILES: Cn1cc(-c2sc3c(C(=O)Nc4cccc(C(F)(F)F)c4)cccc3c2Br)c2c(N)ncnc21]

Compound 7 was prepared by the following steps:

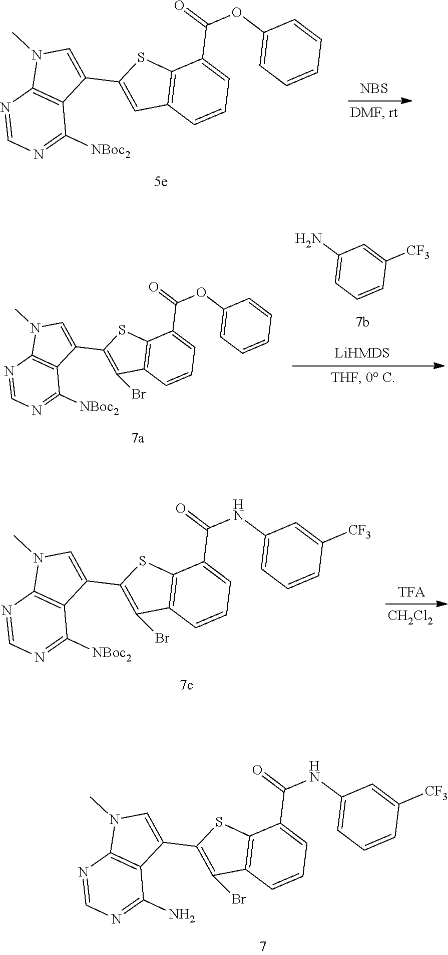

Step 1: To a solution of compound 5e (0.60 g, 1.0 mmol) in DMF (5.0 mL) was added NBS (213.6 mg, 1.2 mmol). The reaction mixture was stirred at room temperature for 12 h, then quenched with saturated aq. Na₂CO₃ (20.0 mL) and Na₂S₂O₃ (20.0 mL), followed by extraction with ethyl acetate (30.0 mL*3). The combined organic phases were washed with saturated brine, dried over anhydrous Na₂SO₄, and concentrated. The resulting residue was purified on an ISCO chromatography (mobile phase: petroleum ether/ethyl acetate) to afford compound 7a as a pale yellow solid (0.55 g, yield: 80%).

Step 2: To a stirred solution of compound 7a (0.15 g, 0.22 mmol) and compound 7b (43.0 mg, 0.27 mmol) in THF (2.0 mL) was added LiHMDS (0.6 mL, 1 M in THF, 0.60 mmol) at 0° C. The reaction mixture was stirred at 0° C. until TLC showed the reaction was completed. The reaction mixture was quenched with saturated aq. Na₂CO₃ (10.0 mL), followed by extraction with a mixed solvent of CH₂Cl₂/MeOH (v/v=10:1, 10.0 mL*3). The combined organic phases were washed with saturated brine (10.0 mL), dried over anhydrous Na₂SO₄, filtered and concentrated to afford crude product 7c, which was used directly in Step 3. ESI-MS (m/z): 746.1 [M+H]⁺.

Step 3: To a solution of compound 7c in CH₂Cl₂ (2.0 mL) was added TFA (2.0 mL). The reaction mixture was stirred at room temperature until TLC showed the reaction was completed. After concentration, the reaction mixture was quenched with saturated aq. Na₂CO₃ (20.0 mL), followed by extraction with a mixed solvent of CH₂Cl₂/MeOH (v/v=10:1, 20.0 mL*3). The combined organic phases were washed with saturated brine (50.0 mL), dried over anhydrous Na₂SO₄, filtered, and concentrated. The resulting residue was purified on an ISCO chromatography (mobile phase: CH₃CN/H₂O) to afford compound 7 as a pale yellow solid (75.0 mg, yield: 62% for two steps). ESI-MS (m/z): 546.3 [M+H]⁺. ¹H NMR (400 MHz, DMSO-d₆) δ 10.86 (s, 1H), 8.40 (d, J=7.3 Hz, 1H), 8.28 (s, 1H), 8.20 (s, 1H), 8.13 (d, J=8.3 Hz, 1H), 8.07 (d, J=7.8 Hz, 1H), 7.83-7.75 (m, 1H), 7.69-7.62 (m, 1H), 7.58 (s, 1H), 7.52 (d, J=7.3 Hz, 1H), 6.35 (br s, 2H), 3.80 (s, 3H).

### Example 8

Compound 8 was prepared by the following steps:

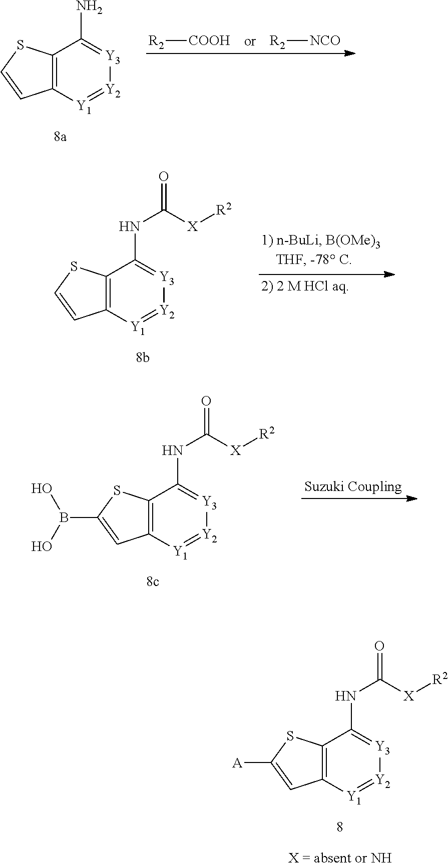

Step 1: Intermediate 8b is prepared by reacting compound 8a with a carboxylic acid or isocyanate (or alternatively, with an intermediate prepared from the reaction of R²—NH₂ and p-nitrophenyl chloroformate).

Steps 2 and 3 follow a synthetic method similar to that used for compound 1 in Example 1. First, n-butyllithium is used to abstract a hydrogen atom to form a carbanion, which then reacts with trimethyl borate. Subsequent hydrolysis yields boronic acid intermediate 8c. Intermediate 8c then undergoes a Suzuki cross coupling reaction with a heteroaromatic halide to afford compound 8.

The structures of compounds 8a-8n are shown in Table 3 below.

TABLE 3

| | | MS | |
|---|---|---|---|
| Cpds | Structures | [M + 1]⁺ | ¹H NMR |
| 8a | 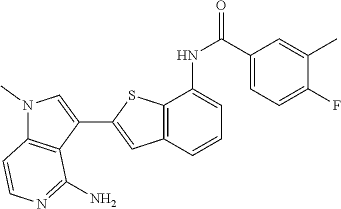 [SMILES: Cc1cc(C(=O)Nc2cccc3cc(-c4cn(C)c5ccnc(N)c45)sc23)ccc1F] | 431.1 | |
| 8b | 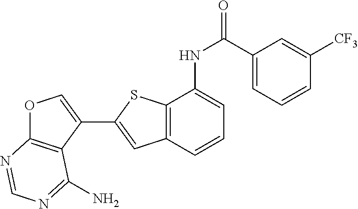 [SMILES: Nc1ncnc2occ(-c3cc4cccc(NC(=O)c5cccc(C(F)(F)F)c5)c4s3)c12] | 455.1 | |
| 8c | 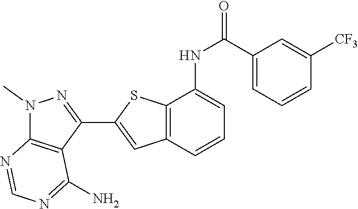 [SMILES: Cn1nc(-c2cc3cccc(NC(=O)c4cccc(C(F)(F)F)c4)c3s2)c2c(N)ncnc21] | 469.1 | |
| 8d | 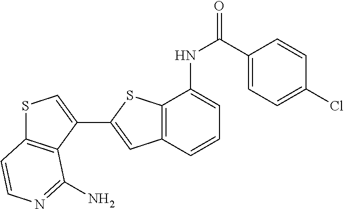 [SMILES: Nc1nccc2scc(-c3cc4cccc(NC(=O)c5ccc(Cl)cc5)c4s3)c12] | 436.0 | |
| 8e | 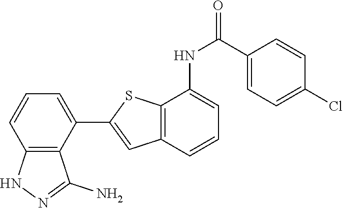 [SMILES: Nc1n[nH]c2cccc(-c3cc4cccc(NC(=O)c5ccc(Cl)cc5)c4s3)c12] | 419.1 | |
| 8f | 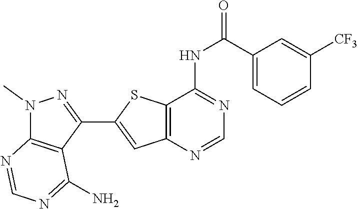 [SMILES: Cn1nc(-c2cc3ncnc(NC(=O)c4cccc(C(F)(F)F)c4)c3s2)c2c(N)ncnc21] | 471.1 | |
| 8g | 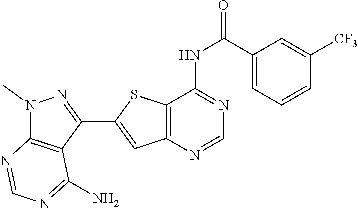 [SMILES: Cn1nc(-c2cc3ncnc(NC(=O)c4cccc(C(F)(F)F)c4)c3s2)c2c(N)ncnc21] | 471.1 | |
| 8h | 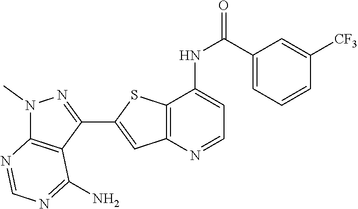 [SMILES: Cn1nc(-c2cc3nccc(NC(=O)c4cccc(C(F)(F)F)c4)c3s2)c2c(N)ncnc21] | 470.1 | |
| 8i | 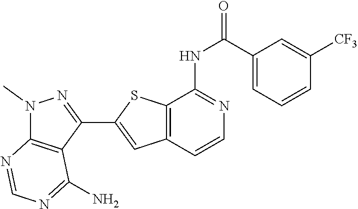 [SMILES: Cn1nc(-c2cc3ccnc(NC(=O)c4cccc(C(F)(F)F)c4)c3s2)c2c(N)ncnc21] | 470.1 | |
| 8j | 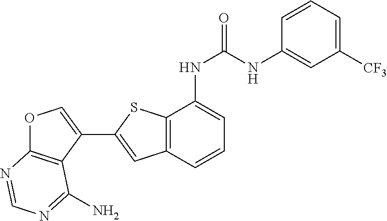 [SMILES: Nc1ncnc2occ(-c3cc4cccc(NC(=O)Nc5cccc(C(F)(F)F)c5)c4s3)c12] | 470.1 | |
| 8k | 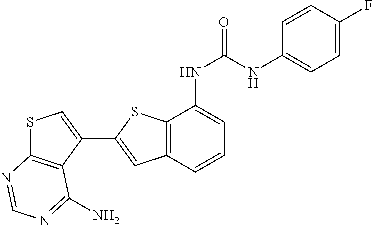 [SMILES: Nc1ncnc2scc(-c3cc4cccc(NC(=O)Nc5ccc(F)cc5)c4s3)c12] | 436.1 | |
| 8l | 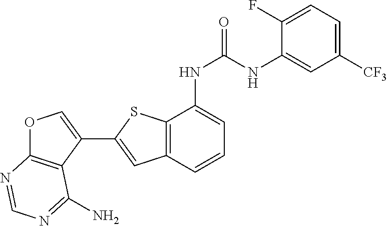 [SMILES: Nc1ncnc2occ(-c3cc4cccc(NC(=O)Nc5cc(C(F)(F)F)ccc5F)c4s3)c12] | 488.1 | |
| 8m | 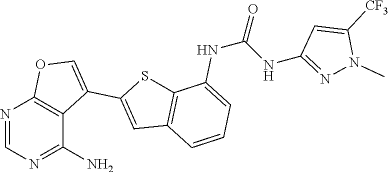 [SMILES: Cn1nc(NC(=O)Nc2cccc3cc(-c4coc5ncnc(N)c45)sc23)cc1C(F)(F)F] | 474.1 | |
| 8n | 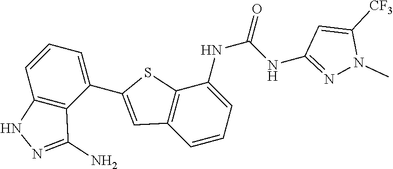 [SMILES: Cn1nc(NC(=O)Nc2cccc3cc(-c4cccc5[nH]nc(N)c45)sc23)cc1C(F)(F)F] | 472.1 | |

### Example 9

### N-(2-((2-aminopyridin-3-yl)amino)benzo[b]thiophen-7-yl)-3-methylbenzamide (compound

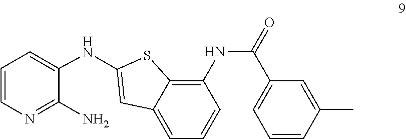 [SMILES: Cc1cccc(C(=O)Nc2cccc3cc(Nc4cccnc4N)sc23)c1]

Compound 9 was prepared by the following steps:

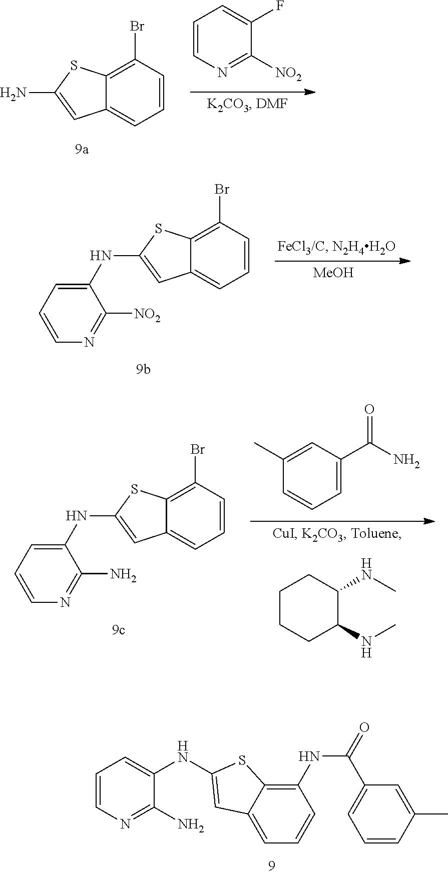

Step 1: To solution of compound 9a (1.00 g, 4.4 mmol) in DM (10 mL was added 3-Fluoro-2-nitropyridine (747 mg, 5.26 mmol) and K₂CO₃ (1.21 g, 8.76 mmol). The resulting mixture was stirred at 80° C. for 3 h, then quenched with saturated aq. NaHCO₃ (100 mL). The mixture was extracted with EtOAc (50 mL*3). The combined organic phases were dried over anhydrous Na₂SO₄, filtrated and concentrated. The resulting residue was purified on an ISCO chromatography to afford compound 9b as a pale yellow solid (1.31 g, yield: 85%). ESI-MS (m/z): 351.2 [M+H]⁺.

Step 2: To a solution of compound 9b (1.31 g, 3.7 mmol) in MeOH (15 mL) was added FeCl₃ (60.4 mg, 0.4 mmol), active carbon (131 mg) and 80% hydrazine hydrate (1.20 g, 18.6 mmol). The reaction mixture was stirred at 60° C. for 6 h. After concentration, the mixture was redissolved in ethyl acetate (20 mL), washed with water (20 mL*2), then dried over Na₂SO₄, filtrated and concentrated. The resulting residue was purified on an ISCO chromatography to afford compound 9c as a pale yellow solid (1.07 g, yield: 90%). ESI-MS (m/z): 321.2 [M+H]⁺.

Step 3: To a solution of compound 9c (200 mg, 0.625 mmol) in toluene (2 mL) was added 3-Methylbenzamide (127 mg, 0.938 mmol), CuI (11.9 mg, 0.063 mmol), (1R,2R)-(−)-N,N′-dimethyl-1,2cyclohexanediamine (17.8 mg, 0.125 mmol) and K₂CO₃ (173 mg, 1.25 mmol). The resulting mixture was degassed with N₂ for three times and stirred at 100° C. for 3 h. The reaction mixture was quenched with saturated aq. NaHCO₃ (10 mL), and then extracted with EtOAc (10 mL*3). The combined organic phases were dried over Na₂SO₄, filtrated and concentrated. The resulting residue was purified on an ISCO chromatography to afford compound 9 as a pale yellow solid (133 mg, yield: 57%). ESI-MS (m/z): 375.5 [M+H]⁺.

### Example 10

### 2-((2-aminopyridin-3-yl)methyl)-N-(4-fluoro-3-methylphenyl)benzo[b]thiophene-7-carboxamide (compound 10)

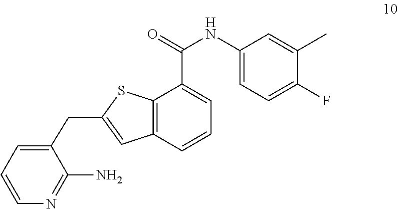 [SMILES: Cc1cc(NC(=O)c2cccc3cc(Cc4cccnc4N)sc23)ccc1F]

Compound 10 was prepared by the following steps:

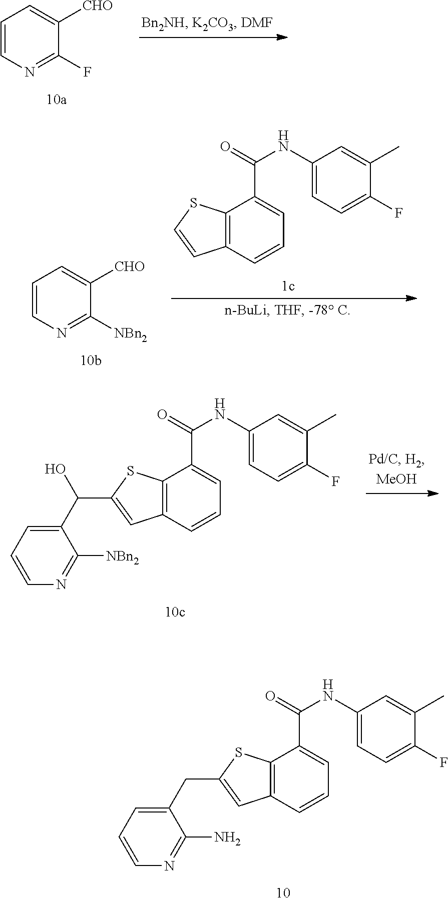

Step 1: To a solution of compound 10a (1.25 g, 10.0 mmol) in DMF (10 mL) was added Dibenzylamine (2.96 g, 15.0 mmol) and K₂CO₃ (2.76 g, 20.0 mmol). The resulting mixture was stirred at 80° C. for 3 h, then quenched by addition of saturated aq. NaHCO₃ (100 mL) and extracted with EtOAc (50 mL*3). The combined organic phases were dried over Na₂SO₄, filtrated and concentrated. The resulting residue was purified on an ISCO chromatography to afford compound 10b as a pale yellow solid (1.66 g, yield: 55%). ESI-MS (m/z): 303.4 [M+H]⁺.

Step 2: To a solution of compound 1c (571 mg, 2.0 mmol) in anhydrous THF (10 mL) was slowly added n-BuLi (2.8 mL, 2.5 M in hexane, 7.0 mmol) dropwise at −78° C., followed by addition of compound 10b (454 mg, 1.5 mmol) after being stirred for 1 h. The resulting mixture was continued stirred for 1.5 h, then warmed to room temperature. The resultant mixture was quenched with saturated aq. NaHCO₃ (30 mL) and extracted with EtOAc (30 mL*3). The combined organic phases were dried over Na₂SO₄, filtrated and concentrated. The resulting residue was purified on an ISCO chromatography to afford compound 10c as a pale yellow solid (300 mg, yield: 34%). ESI-MS (m/z): 588.7 [M+H]⁺.

Step 3: To a solution of compound 10c (100 mg, 0.17 mmol) in MeOH (5 mL) was added Pd/C (10 mg). The resulting mixture was reacted under H₂ atmosphere at 50° C. for 12 h. The reaction mixture was filtrated and concentrated. The resulting residue was purified on an ISCO chromatography to afford compound 10 as a pale yellow solid (27.3 mg, yield: 41%). ESI-MS (m/z): 392.5 [M+H]⁺.

### Example 11

### 2-(2-aminonicotinoyl)-N-(4-fluoro-3-methylphenyl)benzo[b]thiophene-7-carboxamide (compound 11b) and 2-((2-aminopyridin-3-yl)difluoromethyl)-N-(4-fluoro-3-methylphenyl)benzo[b]thiophene-7-carboxamide (compound 11)

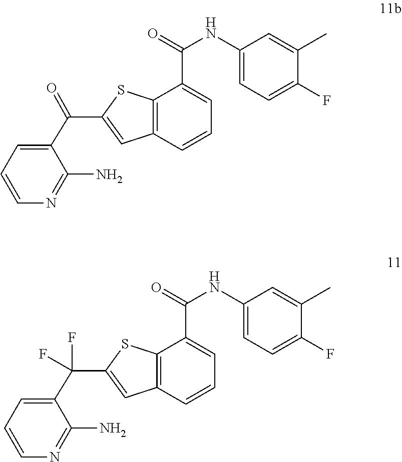 [SMILES: Cc1cc(NC(=O)c2cccc3cc(C(=O)c4cccnc4N)sc23)ccc1F ; Cc1cc(NC(=O)c2cccc3cc(C(F)(F)c4cccnc4N)sc23)ccc1F]

Compound 11b and Compound 11 were prepared by the following step:

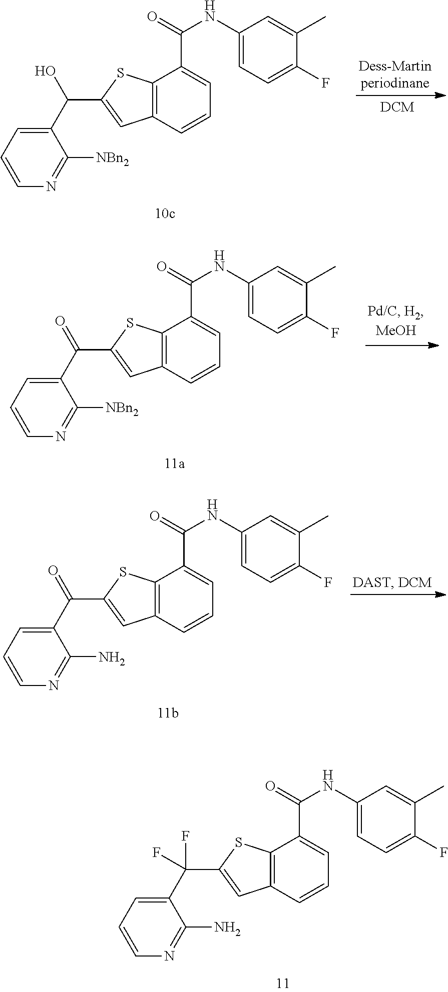

Step 1: To a solution of compound 10c (100 mg, 0.17 mmol) in CH₂Cl₂ (5 mL) was added Dess-Martin periodinane (180 mg, 0.43 mmol). The reaction mixture was filtrated and concentrated after being stirred at room temperature for 2 h. The resulting residue was purified on an ISCO chromatography to afford compound 11a as a pale yellow solid (81.6 mg, yield: 82%). ESI-MS (m/z): 586.7 [M+H]⁺.

Step 2: To a solution of compound 11a (80 mg, 0.14 mmol) in MeOH (3 mL) was added Pd/C (8 mg). The reaction mixture was stirred under H₂ at 50° C. for 12 h, then filtrated and concentrated. The resulting residue was purified on an ISCO chromatography to afford compound 11b as a pale yellow solid (36.9 mg, yield: 65%). ESI-MS (m/z): 406.4 [M+H]⁺.

Step 3: To a solution of compound 11b (50 mg, 0.12 mmol) in CH₂Cl₂ (3 mL) was added DAST (77.4 mg, 0.48 mmol). The resulting mixture was stirred at room temperature for 24 h, followed by addition of saturated aq. NaHCO₃ (10 mL), and then extracted with EtOAc (10 mL*3). The combined organic phases were dried over Na₂SO₄, filtrated and concentrated. The resulting residue was purified on an ISCO chromatography to afford compound 11 as a pale yellow solid (19.5 mg, yield: 38%). ESI-MS (m/z): 428.5 [M+H]+.

### Example 12: CSF-1R, PDGFR-α and KDR (VEGFR2) In Vitro Kinase Activity Assay

### 12.1 Materials and Instruments

SpectraMax i3× Multi-Mode Reader (Molecular Devices)
White 384-well MicroPlate (Cat #264706, Nunc)
HTRF kinEASE TK kit main reagents (Cat #62TKOPEC, Cisbio)
TK-biotin substrate
Streptavidin-XL665
TK Antibody-Cryptate
5× Enzymatic buffer (5×KB)
SEB
HTRF Detection buffer
PDGFRα (Cat #PV4203, Invitrogen)
KDR (Cat #08-191, Carna)
CSF-1R (Cat #PV3249, Invitrogen)
ATP 10 mM (Cat #PV3227, Invitrogen)
DTT 1 M (Cat #D5545, Sigma)
MgCl₂ 1 M (Cat #M8266, Sigma)
MnCl₂ 1 M (Cat #244589, Sigma)

### 12.2 Experimental Procedure

### 12.2.1 Reagent Preparation

5×Substrate-TK and ATP Working Solution: The specific concentrations of Substrate-TK and ATP are shown in Table 4. Dilute Substrate-TK and ATP to 5× of the reaction concentration using 1×Kinase Buffer.

5×Enzyme Working Solution: The kinase concentrations of CSF-1R, PDGFR-α and KDR are shown in Table 4. Prepare the 5×enzyme working solution using 1×Kinase Buffer.

4×Sa-XL665Working Solution: The concentration of Sa-XL665 in the reaction is shown in Table 4. Prepare the 4×Sa-XL665 working solution using Detection Buffer.

4× TK-Ab-cryptate Working Solution: Dilute TK-Ab-Cryptate 100 times using Detection Buffer to prepare the working solution.

TABLE 4
Composition and Concentration Table of Each
Component in the Kinase Reaction System

| | Final Concentration in the | | | | | | | | Final Concentration in the | | |
|---|---|---|---|---|---|---|---|---|---|---|---|
| | Kinase Reaction Step (10 μL) | | | | | | | | Total Reaction (20 μL) | | |
| | | | | | | | Enzyme | | | | TK |
| | Enzyme | | | | TK- | | Reaction | | Streptavidin- | | Antibody- |
| | Concentration | | ATP | | substrate | | Time | | XL665 | | Cryptate |
| CSF-1R | 10 | ng/well | 5 | μM | 1 | μM | 60 | min | 125 | nM | 1:100 dilution |
| PDGFR-α | 0.8 | ng/well | 5 | μM | 1 | μM | 40 | min | 125 | nM | 1:100 dilution |
| KDR | 0.1 | ng/well | 0.2 | μM | 0.2 | μM | 30 | min | 25 | nM | 1:100 dilution |

TABLE 5
Components of 1 mL 1× Kinase Buffer (μL)

| | 5× Enzyme | | | | | |
|---|---|---|---|---|---|---|
| Kinase | buffer | MgCl₂ | MnCl₂ | DTT | SEB | ddH₂O |
| CSF-1R | 200 | 5 | 1 | 1 | 20 | 773 |
| PDGFR-α | 200 | 5 | 1 | 1 | 8 | 785 |
| KDR | 200 | 5 | 1 | 1 | 10 | 783 |

### 12.2.2 Experimental Procedure

After all reagents were prepared according to the methods described above, except for the enzyme, they were equilibrated to room temperature before starting the sample addition.

a) First, 2.5% DMSO solutions were prepared separately using the pre-prepared 1× kinase buffer (DMSO concentrations that were too high could affect the reaction, so the final concentration of DMSO was controlled at 1%). Then, the corresponding 2.5% DMSO solution for the enzyme was used to dilute the test compounds. Compounds were screened with two concentrations of 100 nM and 10 nM. Except for the control wells, 4 μL of the diluted test compound solution was added to the reaction wells, and 4 μL of the pre-prepared 2.5% DMSO solution was added to the control wells.

b) 2 μL of the pre-prepared TK-biotin substrate solution at the corresponding concentration for the kinase was added to all reaction wells.

c) 2 μL of the pre-prepared enzyme solution at the corresponding concentration was added to all reaction wells except the negative control wells. For the negative control wells, 2 μL of 1× kinase buffer corresponding to the enzyme was added to make up the volume. The plate was sealed with sealing film, mixed gently, and incubated at room temperature for 10 min to allow the compounds to fully interact with the enzyme.

d) 2 μL of the ATP solution at the corresponding concentration for the enzyme was added to all reaction wells to initiate the kinase reaction. ATP concentrations and reaction times for enzyme screening are shown in Table 4.

e) Five minutes before the end of the kinase reaction, the detection solution was prepared. The detection buffer from the kit was used to prepare detection solutions for Streptavidin-XL665 and TK antibody europium cryptate (1:100) at the appropriate concentrations for the two enzymes.

f) After the kinase reaction was completed, 5 μL of the diluted Streptavidin-XL665 solution was added to all reaction wells. After mixing, the diluted TK antibody europium cryptate detection solution was immediately added.

g) The plate was sealed and mixed, incubated at room temperature for 1 hour, and the fluorescence signal was detected using the SpectraMax i3× Multi-Mode Reader (Molecular Devices). The inhibition rate for each well was calculated based on the full-activity control wells and background signal wells, and the average value of the replicates was taken.

The experimental pipetting scheme is summarized in Table 6:

TABLE 6

| | Kinase | | |
|---|---|---|---|
| Enzyme Step | Assay | Control | |
| (10 μL) | Sample | Negative | Positive |
| Compound | 4 μL | 4 μL 2.5% DMSO/ | 4 μL 2.5% DMSO/ |
| | | kinase buffer | kinase buffer |
| TK Substrate-biotin | 2 μL | 2 μL | 2 μL |
| Kinase | 2 μL | 2 μL Kinase buffer | 2 μL |

Seal the plate, incubate at room temperature for 10 min

| | | | |
|---|---|---|---|
| ATP | 2 μL | 2 μL | 2 μL |

Seal the plate, incubate at room temperature
Detection Step (10 μL)

| | | | |
|---|---|---|---|
| Sa-XL665 | 5 μL | 5 μL | 5 μL |
| TK Ab-Cryptate | 5 μL | 5 μL | 5 μL |

Seal the plate, incubate at room temperature for 1 hour
Excitation at 320 nm, emission at 665 nm and 615 nm

### 12.3 Data Analysis

$$Emission Rate ( ER ) = 665 nm Emission signal / 615 nm Emission signal$$

$$Inhibition rate = ( {ER}_{positive} - {ER}_{sample} ) / ( {ER}_{positive} - {ER}_{negative} ) * 100 \%$$

### Example 13: DDR1 In Vitro Kinase Activity Assay

### 13.1 Materials and Instruments

Plate reader (I3×, MD)
White 384-well MicroPlate (Cat #6007290, PerkinElmer)
5× Kinase Buffer A (Cat #PV3189, Thermo)
Kinase Tracer 178 (Cat #PV5593, Thermo)
LanthaScreen® Eu-anti-GST Antibody (Cat #PV5594, Thermo)
DDR1 (Cat #10730-H20B1, Sino Biological)

### 13.2 Experimental Procedure

### 13.2.1 Reagent Preparation

The compounds to be tested were tested at two concentrations of 100 nM and 10 nM.

Kinase/Antibody Mixtures Preparation: Dilute Kinase/Antibody Mixtures with 1×Kinase Buffer to form 3× working solutions with final concentrations of 25 nM kinase and 2 nM Eu-Anti-GST Antibody, respectively.

Antibody Working Solution Preparation: Dilute the Antibody with 1×Kinase Buffer to prepare a 3×working solution, resulting in a final concentration of 2 nM.

Kinase Tracer 178 Working Solution Preparation: Dilute Kinase Tracer 178 with 1×Kinase Buffer to make a 3× working solution with a final concentration of 10 nM.

TABLE 7
Composition and Concentration Table of Each
Component in the Kinase Reaction System

| | Final Concentration in the Kinase Reaction Step (15 μL) | | | |
|---|---|---|---|---|
| | | | Kinase | Enzyme |
| | Enzyme | LanthaScreen ® Eu- | Tracer | Reaction |
| | Concentration | anti-GST Antibody | 178 | Time |
| DDR1 | 25 nM | 2 nM | 10 nM | 60 min |

### 13.2.2 Experimental Procedure

After all reagents were prepared according to the methods described above, except for the enzyme, they were equilibrated to room temperature before starting the sample addition.

a) First, 3% DMSO solutions were prepared separately using the pre-prepared 1× kinase buffer (DMSO concentrations that were too high could affect the reaction, so the final concentration of DMSO was controlled at 1%). Then, the corresponding 3% DMSO solution for the enzyme was used to dilute the test compounds. Compounds were screened with two concentrations of 100 nM and 10 nM. Except for the control wells, 5 μL of the diluted test compound solution was added to the reaction wells, and 5 μL of the pre-prepared 3% DMSO solution was added to the control wells.

b) 5 μL of the pre-prepared Kinase/Antibody mixtures was added to all reaction wells except the negative control wells. For the negative control wells, 5 μL of the previously prepared Antibody solution was added.

c) 5 μL of the pre-prepared Kinase Tracer 178 solution was added to all reaction wells.

d) The plate was sealed and mixed, incubated at room temperature for 1 hour, and the fluorescence signal was detected using the Plate reader (I3×, MD) instrument (excitation at 340 nm, emission at 665 nm and 615 nm). The inhibition rate for each well was calculated based on the full-activity control wells and background signal wells, and the average value of the replicates was taken.

The experimental pipetting scheme is summarized in Table 8:

TABLE 8

| | Kinase | | |
|---|---|---|---|
| Enzyme Step | Assay | Control | |
| (10 μL) | Sample | Negative | Positive |
| Compounds | 5 μL | 5 μL 3% DMSO/ | 5 μL 3% DMSO/ |
| | | kinase buffer | kinase buffer |
| Kinase/Antibody | 5 μL | 5 μL Antibody | 5 μL |
| Mixtures | | solution | |
| Kinase Tracer 178 | 5 μL | 5 μL | 5 μL |

Seal the plate, incubate at room temperature for 1 h
Excitation at 340 nm, emission at 665 nm and 615 nm

### 13.3 Data Analysis

$$Emission Rate ( ER ) = 665 nm Emission signal / 615 nm Emission signal$$

$$Inhibition rate = ( {ER}_{positive} - {ER}_{sample} ) / ( {ER}_{positive} - {ER}_{negative} ) * 100 \%$$

TABLE 9

| | CSF-1R | | PDGFR α | | VEGFR2 | | DDR1 | |
|---|---|---|---|---|---|---|---|---|
| Cpds | 100 nM | 10 nM | 100 nM | 10 nM | 100 nM | 10 nM | 100 nM | 10 nM |
| 1 | 70.0 | 24.0 | 59.3 | 8.2 | 45.6 | 29.6 | 81.0 | 9.4 |
| 2 | 69.3 | 17.8 | 98.5 | 95.0 | 95.7 | 81.4 | 93.7 | 62.7 |
| 4ap | 88.1 | 50.6 | 73.8 | 73.1 | 89.0 | 76.6 | 97.0 | 63.5 |
| 4ag | 98.1 | 82.2 | 96.2 | 68.0 | 100.5 | 79.0 | 89.8 | 63.7 |
| 4ah | 97.2 | 86.1 | 97.6 | 59.2 | 97.3 | 78.5 | 85.3 | 59.2 |
| 4ak | — | — | 32.0 | 33.4 | — | — | 23.8 | 19.6 |
| 4aq | 69.5 | 29.3 | 100.1 | 69.2 | 97.8 | 94.0 | 61.8 | 9.5 |
| 4as | 88.2 | 37.4 | 80.8 | 32.6 | 97.5 | 83.0 | 78.4 | 33.4 |
| 4at | 96.7 | 83.6 | 94.6 | 77.3 | 92.2 | 91.4 | 82.7 | 49.7 |
| 4bj | 94.9 | 58.7 | 96.1 | 71.7 | — | — | — | — |
| 4bp | 86.0 | 36.9 | 88.2 | 10.6 | — | — | — | — |
| 5 | 84.0 | 37.0 | 97.7 | 92.3 | — | — | — | — |
| 6aa | 96.4 | 48.2 | 99.0 | 83.0 | 101.6 | 96.8 | 91.9 | 74.7 |
| 6ab | 97.8 | 79.5 | 96.7 | 59.3 | 98.1 | 98.7 | 97.7 | 89.9 |
| 6ac | 96.6 | 81.6 | 100.9 | 75.7 | 98.4 | 98.8 | 96.5 | 93.7 |
| 6ad | 85.3 | 34.2 | 97.2 | 93.4 | — | — | — | — |
| 6ae | 94.1 | 83.9 | 96.8 | 72.3 | — | — | — | — |
| 6af | 86.9 | 63.1 | 97.6 | 92.3 | — | — | — | — |
| 6ag | 84.5 | 67.9 | 99.2 | 82.8 | — | — | — | — |
| 7 | 74.3 | 4.5 | 85.5 | 37.3 | — | — | — | — |

### Example 14: In Vitro Proliferation Activity Assay of M-NFS-60 Cells

### 14.1 Materials and Instruments

M-NFS5-60 cell line (ATCC, Cat #CRL-183 8)
RPMI1640 medium (Gibco, Cat #C11875500BT)
Recombinant Mouse CSF 1/M-CSF (Sino Biological Inc., Cat #51112-MNAH)
PAN/Serum (BioTECH, Cat #P30-2602)
Trypsin/0.05% o Trypsin-EDTA (Gibco, Cat #25300-062)
Penicillin-Streptomycin (Hyclone, Cat #SV30010)
DMSO (Solarbio, Cat #D8371)
PBS (Solarbio, Cat #P1020)
CCK (AbMole BioScience, Cat #M4839-50OTests)
384 Well Cell Culture Plate (NEST, Cat #761001)
Full-wavelength reader/microplate reader (Multiskan GO)

### 14.2 Experimental Purpose

### 14.2.1 Reagent Preparation

M-CSF factor stock solution: take 10 g of powdered M-CSF factor, add 0.4 mL of saline, formulate 25 g/mL and store at −80° C.

$$M - CSF factor ( 25 ng / ml ) medium : RPMI 1640 + 10 \% PAN + 100 \times Penicillin - Streptomycin + 100 \times M - CSF stock solution .$$

Preparation of Compound Stock Solution: accurately weigh 2-3 mg of the substance, add an appropriate amount of DMSO, vortex to mix thoroughly, prepare a 30 mM stock solution, and store it in a −80° C. refrigerator.

### 14.2.2 Experimental Procedure

a) Harvest cells in the logarithmic growth phase, count them, and resuspend the cells in complete medium containing M-CSF factor to an appropriate density. Seed the cells into a 384-well plate at 2,500 cells per well. Incubate the plate for 4 hours in a humidified incubator at 37° C., 100% relative humidity, and 5% CO₂.

b) Dilute the test compounds to the desired working concentrations in the same complete medium. Add 4 μL of the diluted compound solution per well to the cells. The final test concentrations of the compounds should start from 500 nM, followed by a 3-fold serial dilution, for a total of 7 concentration points.

c) Incubate the cell plate for 48 hours in a humidified incubator at 37° C., 100% relative humidity, and 5% CO₂.

d) Add 4 μL of CCK-8 reagent directly to the cell culture medium in each well. Return the plate to the 37° C. incubator and incubate for 1 to 4 hours.

The experimental pipetting scheme is summarized in Table 10.

TABLE 10

| | Groups | | | | |
|---|---|---|---|---|---|
| | | DMSO | | Experimental | |
| Step | Blank control | control well | | well | |
| Cell suspension | 40 μL (M-CSF | 36 | μL | 36 | μL |
| | medium) | | | | |
| Compound | — | 4 μL (M-CSF | | 4 | μL |
| solution | | medium) | | | |
| CCK | 4 μL | 4 | μL | 4 | μL |

### 14.3 Data Analysis

The formula for calculating the inhibition rate is provided below, and the analysis results are presented in Table 11.

$$Inhibition rate = ( OD value of the DMSO well - OD value of the experimental \text{well)/(} OD value of the DMSO well - OD value of the blank control ) \times 100 \% .$$

TABLE 11

| Cpds | M-NFS-60 cell assay IC₅₀ (nM) |
|---|---|
| 1 | *** |
| 2 | **** |
| 3 | * |
| 4aa | * |
| 4ab | * |
| 4ac | * |
| 4ad | * |
| 4ag | **** |
| 4ah | **** |
| 4ai | *** |
| 4ak | * |
| 4al | ** |
| 4am | * |
| 4an | * |
| 4ao | * |
| 4ap | **** |
| 4aq | **** |
| 4ar | **** |
| 4as | **** |
| 4at | **** |
| 4bh | * |
| 4bi | **** |
| 4bj | **** |
| 4bp | **** |
| 5 | *** |
| 6aa | **** |
| 6ab | **** |
| 6ac | **** |
| 6ad | **** |
| 6ae | **** |
| 6af | **** |
| 6ag | **** |
| 6al | * |
| 7 | *** |

Note:
****: ≤50 nM;
***: >50 nM, <200 nM;
**: ≥200 nM, ≤500 nM;
*: >500 nM

1-41. (canceled)

42. A compound or a pharmaceutically acceptable salt of Formula (III) or a stereoisomer or an isotopologue thereof, 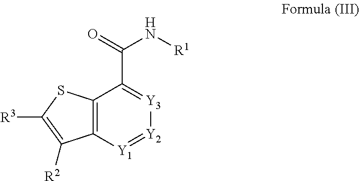
wherein, Y₁ is CR^{Y1};
Y₂ is CR^{Y2};
Y₃ is CR^{Y3};
R¹ is substituted or unsubstituted C₁-C₆ alkyl, substituted or unsubstituted C₃-C₁₀ cycloalkyl, substituted or unsubstituted C₆-C₁₀ cycloalkenyl, substituted or unsubstituted 4-10 membered heterocycloalkyl, substituted or unsubstituted 6-10 membered heterocycloalkenyl, substituted or unsubstituted C₆-C₁₀ aryl, or substituted or unsubstituted 5-10 membered heteroaryl;
R² is hydrogen, deuterium, halogen, hydroxy, cyano, —NR^{a}R^{b}, substituted or unsubstituted C₁-C₆ alkyl, substituted or unsubstituted C₁-C₆ alkoxy, substituted or unsubstituted C₃-C₁₀ cycloalkyl, substituted or unsubstituted C₂-C₆ alkenyl, or substituted or unsubstituted C₂-C₆ alkynyl;
R³ is substituted or unsubstituted heteroaryl;
wherein, R^{Y1}, R^{Y2} and R^{Y3} each independently represent hydrogen, deuterium, halogen, cyano, nitro, substituted or unsubstituted C₁-C₆ alkyl, substituted or unsubstituted C₁-C₆ alkoxy, substituted or unsubstituted C₂-C₆ alkenyl, substituted or unsubstituted C₂-C₆ alkynyl, —OR^{a}, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}COR^{b}, —CONR^{a}R^{b}, substituted or unsubstituted C₃-C₆ cycloalkyl, substituted or unsubstituted C₃-C₆ heterocycloalkyl, substituted or unsubstituted C₆-C₁₀ aryl, or 2 substituted or unsubstituted 5-10 membered heteroaryl;
wherein R^{a} and R^{b} are independently hydrogen, deuterium, halogen, cyano, hydroxy, amino, substituted or unsubstituted C₁-C₆ alkyl, substituted or unsubstituted C₁-C₆ alkoxy, or substituted or unsubstituted C₃-C₁₀ cycloalkyl; or R^{a} and R^{b} together with the atom to which they are attached form a 3-6 membered ring, and the said ring contains 1 or 2 heteroatoms selected from O, N, S, and P, and further, the said ring may optionally be substituted by substituents selected from halogen, hydroxy, and C₁-C₆ alkyl.

43. The compound or the pharmaceutically acceptable salt of Formula (III) or the stereoisomer or isotopologue thereof according to claim 42, wherein R^{Y1}, R^{Y2} and R^{Y3} are independently selected from hydrogen, deuterium, fluoro, chloro, bromo, iodo, amino, hydroxy, cyano, methyl, ethyl, n-propyl, isopropyl, n-butyl, isobutyl, sec-butyl, tert-butyl, methoxy, ethoxy, fluoromethyl, fluoroethyl, cyclopropyl, cyclobutyl, cyclopentyl, and cyclohexyl.

44. The compound or the pharmaceutically acceptable salt of Formula (III) or the stereoisomer or isotopologue thereof according to claim 42, wherein R¹ is C₁-C₆ alkyl, C₃-C₁₀ cycloalkyl, C₆-C₁₀ cycloalkenyl, 4-10 membered heterocycloalkyl, 6-10 membered heterocycloalkenyl, C₆-C₁₀ aryl, or 5-10 membered heteroaryl;
and the C₁-C₆ alkyl, C₃-C₁₀ cycloalkyl, C₆-C₁₀ cycloalkenyl, 4-10 membered heterocycloalkyl, 6-10 membered heterocycloalkenyl, C₆-C₁₀ aryl, and 5-10 membered heteroaryl are optionally substituted with at least one substituent independently selected from hydrogen, deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}COR^{b}, —CONR^{a}R^{b}, C₃-C₆ cycloalkyl, C₃-C₆ heterocycloalkyl, C₆-C₁₀ aryl, 5-10 membered heteroaryl, C₁-C₆ alkyl substituted with R¹¹, C₁-C₆ alkoxy substituted with R¹¹, C₂-C₆ alkenyl substituted with R¹¹, C₂-C₆ alkynyl substituted with R¹¹, C₃-C₆ cycloalkyl substituted with R¹¹, C₃-C₆ heterocycloalkyl substituted with R¹¹, C₆-C₁₀ aryl substituted with R¹¹, and 5-10 membered heteroaryl substituted with R¹¹;
wherein R¹¹ at each occurrence is independently selected from deuterium, halogen, cyano, nitro, C₁-C₆ alkyl, C₁-C₆ alkyl substituted with R¹², C₁-C₆ alkoxy, C₁-C₆ alkoxy substituted with R¹², C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}COR^{b}, —CONR^{a}R^{b}, C₃-C₆ cycloalkyl, C₃-C₆ cycloalkyl substituted with R¹², C₃-C₆ heterocycloalkyl, C₃-C₆ heterocycloalkyl substituted with R¹², C₆-C₁₀ aryl, C₆-C₁₀ aryl substituted with R¹², 5-10 membered heteroaryl, and 5-10 membered heteroaryl substituted with R¹²;
R¹² at each occurrence is independently selected from deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, and C₃-C₆ cycloalkyl.

45. The compound or the pharmaceutically acceptable salt of Formula (III) or the stereoisomer or isotopologue thereof according to claim 42, wherein R¹ is substituted or unsubstituted C₃-C₁₀ cycloalkyl, substituted or unsubstituted 4-10 membered heterocycloalkyl, substituted or unsubstituted C₆-C₁₀ aryl, or substituted or unsubstituted 5-10 membered heteroaryl.

46. The compound or the pharmaceutically acceptable salt of Formula (III) or the stereoisomer or isotopologue thereof according to claim 42, wherein R¹ is substituted or unsubstituted C₃-C₁₀ cycloalkyl, substituted or unsubstituted C₆-C₁₀ aryl, or substituted or unsubstituted 5-10 membered heteroaryl.

47. The compound or the pharmaceutically acceptable salt of Formula (III) or the stereoisomer or isotopologue thereof according to claim 42, wherein R¹ is substituted or unsubstituted C₆-C₁₀ aryl or substituted or unsubstituted 5-10 membered heteroaryl.

48. The compound or the pharmaceutically acceptable salt of Formula (III) or the stereoisomer or isotopologue thereof according to claim 42, wherein R² is hydrogen, deuterium, halogen, hydroxy, amino, cyano, C₂-C₆ alkenyl, C₂-C₆ alkynyl, C₁-C₆ alkyl, or C₃-C₁₀ cycloalkyl; and the C₁-C₆ alkyl and C₃-C₁₀ cycloalkyl are optionally substituted with at least one substituent independently selected from deuterium, halogen, hydroxy, amino, cyano, C₂-C₆ alkenyl, C₂-C₆ alkynyl, C₁-C₆ alkyl, and C₃-C₁₀ cycloalkyl.

49. The compound or the pharmaceutically acceptable salt of Formula (III) or the stereoisomer or isotopologue thereof according to claim 42, wherein R³ is substituted or unsubstituted 5-10 membered heteroaryl.

50. The compound or the pharmaceutically acceptable salt of Formula (III) or the stereoisomer or isotopologue thereof according to claim 49, wherein R³ is substituted or unsubstituted 5- or 6-membered heteroaryl or substituted or unsubstituted 8-10 membered heteroaryl.

51. The compound or the pharmaceutically acceptable salt of Formula (III) or the stereoisomer or isotopologue thereof according to claim 49, wherein R³ is substituted or unsubstituted heteroaryl containing 1-5 heteroatoms selected from N, O, and S.

52. The compound or the pharmaceutically acceptable salt of Formula (III) or the stereoisomer or isotopologue thereof according to claim 49, wherein R³ is 5-10 membered heteroaryl or 5-10 membered heteroaryl substituted with at least one substituent independently selected from hydrogen, deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}C(O)R^{b}, —C(O)NR^{a}R^{b}, C₃-C₆ cycloalkyl, C₃-C₆ heterocycloalkyl, C₆-C₁₀ aryl, 5-10 membered heteroaryl, C₁-C₆ alkyl substituted with R³¹, C₁-C₆ alkoxy substituted with R³¹, C₂-C₆ alkenyl substituted with R³¹, C₂-C₆ alkynyl substituted with R³¹, C₃-C₆ cycloalkyl substituted with R³¹, C₃-C₆ heterocycloalkyl substituted with R³¹, C₆-C₁₀ aryl substituted with R³¹, and 5-10 membered heteroaryl substituted with R³¹;
wherein, R³¹ at each occurrence is independently selected from deuterium, halogen, cyano, nitro, C₁-C₆ alkyl, C₁-C₆ alkyl substituted with R³², C₁-C₆ alkoxy, C₁-C₆ alkoxy substituted with R³², C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}COR^{b}, —CONR^{a}R^{b}, C₃-C₆ cycloalkyl, C₃-C₆ cycloalkyl substituted with R³², C₃-C₆ heterocycloalkyl, C₃-C₆ heterocycloalkyl substituted with R³², C₆-C₁₀ aryl, C₆-C₁₀ aryl substituted with R³², 5-10 membered heteroaryl, and 5-10 membered heteroaryl substituted with R³²;
R³² at each occurrence is independently selected from deuterium, halogen, cyano, nitro, C₁-C₆ alkyl, C₁-C₆ alkoxy, C₂-C₆ alkenyl, C₂-C₆ alkynyl, —OR^{a}, oxo, —NR^{a}R^{b}, —SR^{a}, —SO₃R^{a}, —S(O)₂R^{a}, —S(O)(═NR^{b})R^{a}, —S(O)R^{a}, —SF₅, —P(O)R^{a}R^{b}, —C(O)R^{a}, —C(O)OR^{a}, —OC(O)R^{a}, —OC(O)NR^{a}R^{b}, —NR^{a}COR^{b}, —CONR^{a}R^{b}, C₃-C₆ cycloalkyl, and —Si(R^{a})₃.

53. The compound or the pharmaceutically acceptable salt of Formula (III) or the stereoisomer or isotopologue thereof according to claim 49, wherein R³ is 5-10 membered heteroaryl or 5-10 membered heteroaryl substituted with at least one substituent selected from hydrogen, deuterium, halogen, cyano, nitro, amino, hydroxy, C₁-C₆ alkyl, C₁-C₆ alkoxy, C₃-C₆ cycloalkyl, C₃-C₆ heterocycloalkyl, C₆-C₁₀ aryl, 5-10 membered heteroaryl, C₁-C₆ haloalkyl, C₁-C₆ haloalkoxy, C₃-C₆ halocycloalkyl, hydroxy-substituted C₁-C₆ alkyl, C₃-C₆ heterocycloalkyl-substituted C₁-C₆ alkyl, —NR^{a}R-substituted C₁-C₆ alkyl, —NR^{a}R-substituted C₁-C₆ alkoxy, C₃-C₆ heterocycloalkyl-substituted C₁-C₆ alkoxy, C₁-C₆ alkoxy-substituted C₃-C₆ heterocycloalkyl, C₃-C₆ heterocycloalkyl substituted with hydroxy-substituted C₁-C₆ alkyl, C₁-C₆ alkyl substituted with —Si(R^{a})₃-substituted C₁-C₆ alkoxy, C₁-C₆ alkyl-substituted 5-10 membered heteroaryl, and 5-10 membered heteroaryl substituted with hydroxy-substituted C₁-C₆ alkyl.

54. The compound or the pharmaceutically acceptable salt of Formula (III) or the stereoisomer or isotopologue thereof according to claim 52, wherein R³ is substituted or unsubstituted

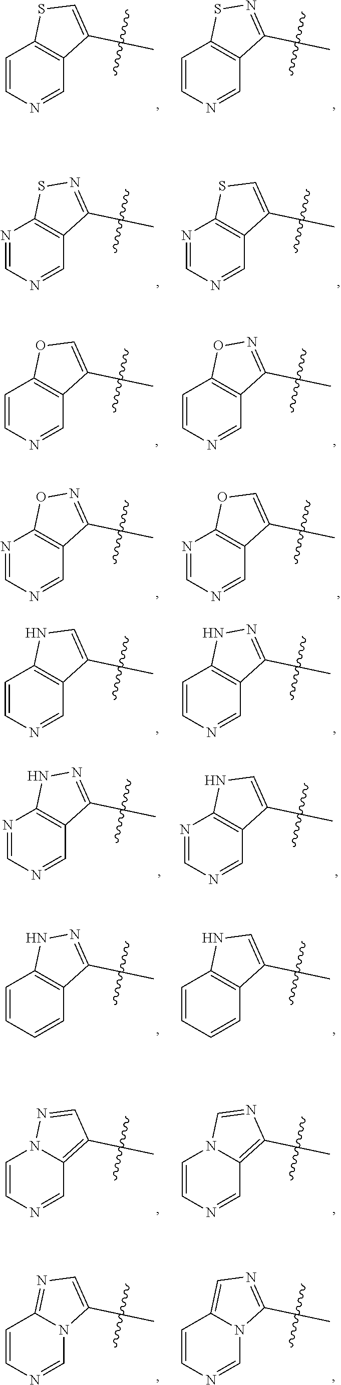

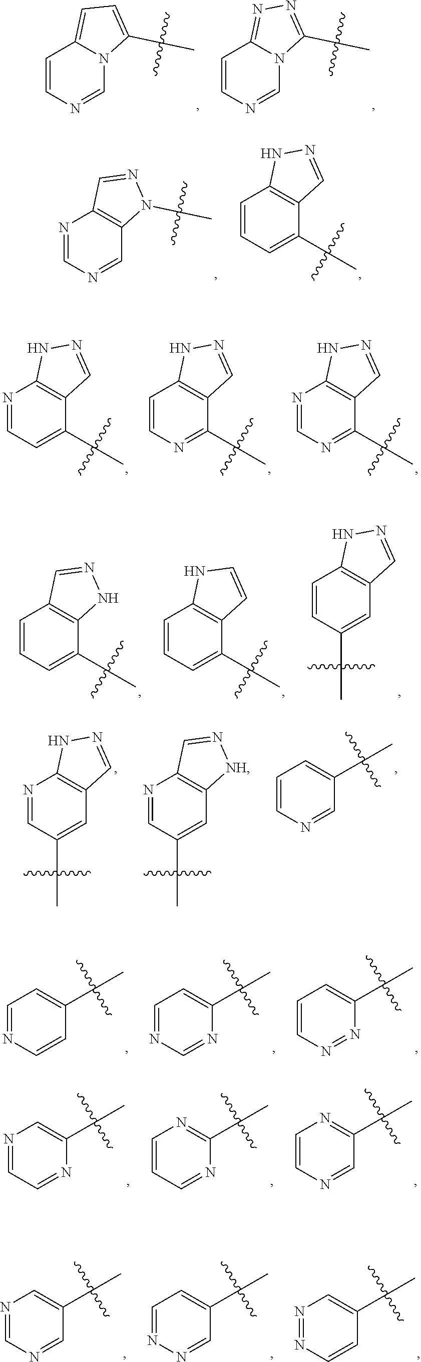

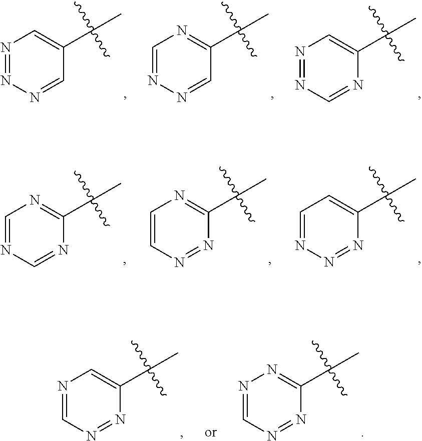

55. The compound or the pharmaceutically acceptable salt of Formula (III) or the stereoisomer or isotopologue thereof according to claim 49, wherein R³ is a substituted or unsubstituted 8-10 membered heteroaryl containing at least one nitrogen atom and containing one sulfur or oxygen atom.

56. The compound or the pharmaceutically acceptable salt of Formula (III) or the stereoisomer or isotopologue thereof according to claim 55, wherein R³ is a substituted or unsubstituted 9-membered heteroaryl containing 1 to 4 nitrogen atoms and containing one sulfur or oxygen atom.

57. The compound or the pharmaceutically acceptable salt of Formula (III) or the stereoisomer or isotopologue thereof according to claim 49, wherein R³ is substituted or unsubstituted

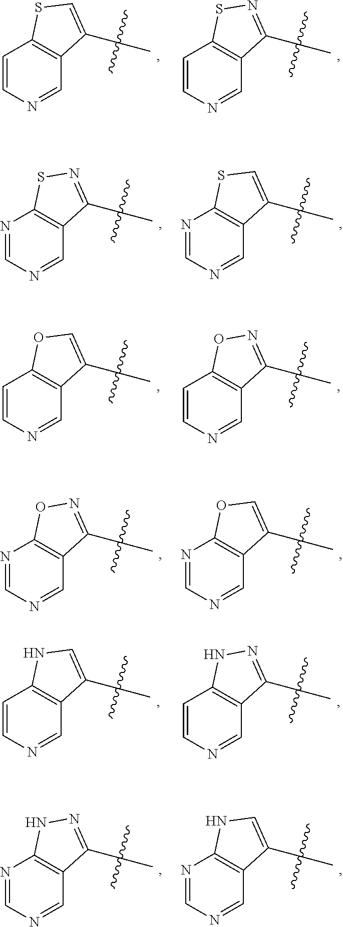

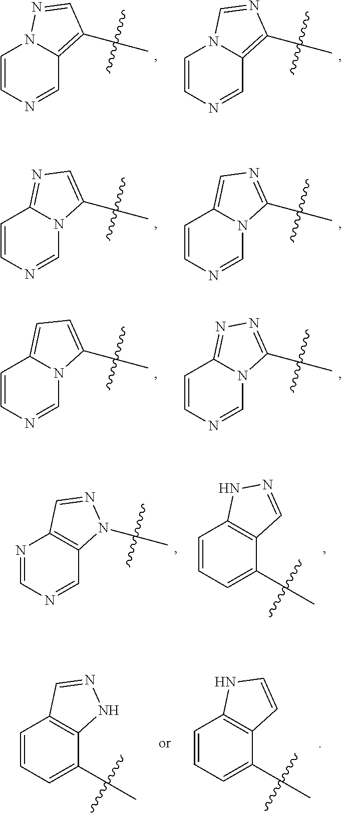

58. The compound or the pharmaceutically acceptable salt of Formula (III) or the stereoisomer or isotopologue thereof according to claim 49, wherein R³ is substituted or unsubstituted 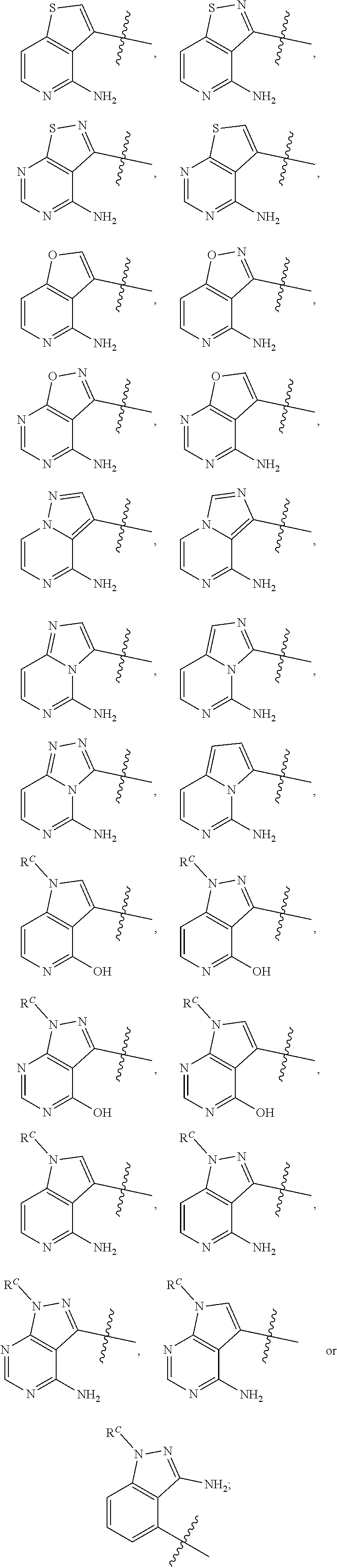
wherein R^{c} is hydrogen, C₁-C₆ alkyl, C₁-C₆ alkoxy, C₃-C₁₀ cycloalkyl, hydroxy-substituted C₁-C₆ alkyl, C₃-C₆ heterocycloalkyl-substituted C₁-C₆ alkyl, C₃-C₆ heterocycloalkyl-substituted C₁-C₆ alkoxy, C₁-C₆ alkyl-substituted C₃-C₆ heterocycloalkyl, —NR^{a}R-substituted C₁-C₆ alkyl, or —NR^{a}R-substituted C₁-C₆ alkoxy.

59. The compound or the pharmaceutically acceptable salt of Formula (III) or the stereoisomer or isotopologue thereof according to claim 49, wherein R³ is

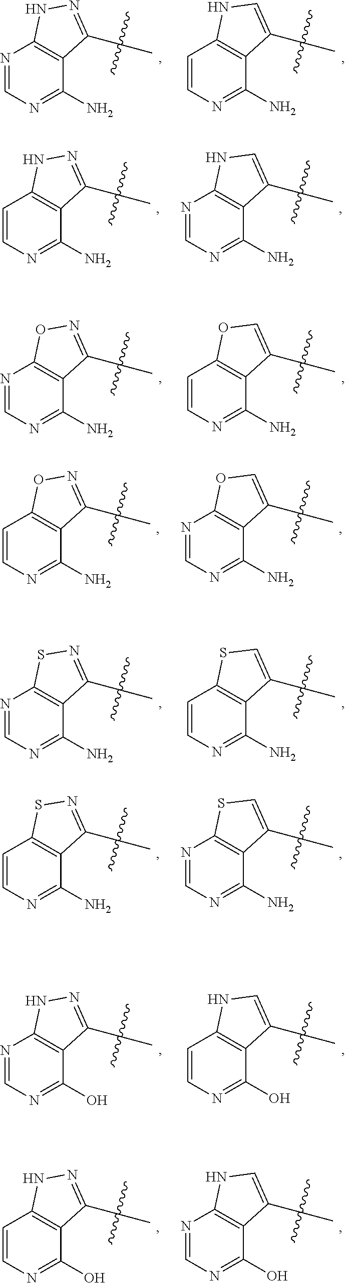

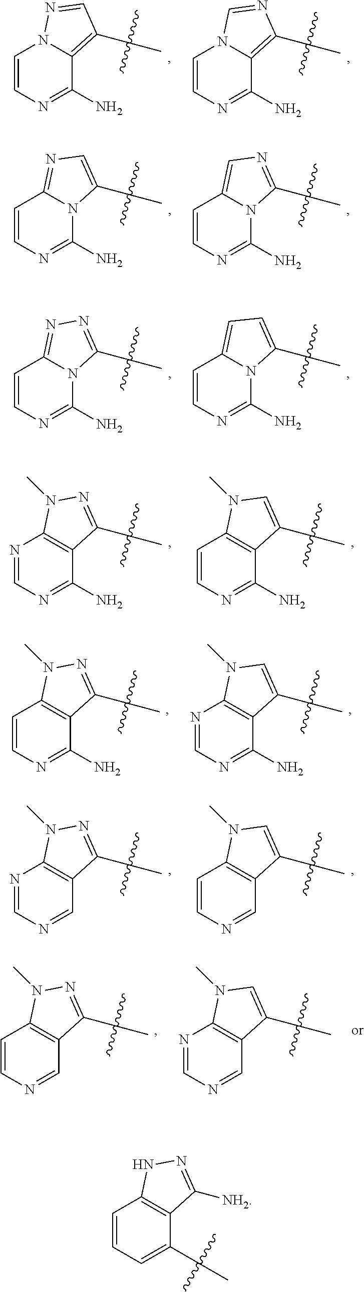

60. A compound or a pharmaceutically acceptable salt thereof selected from any one of the below or a stereoisomer thereof:

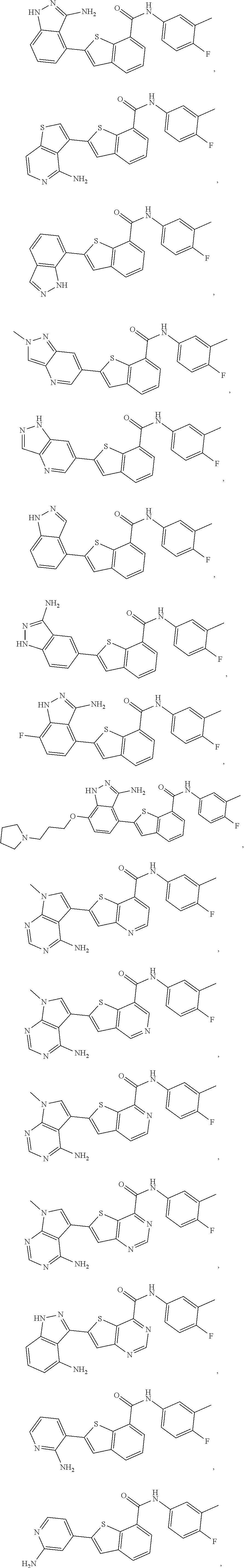

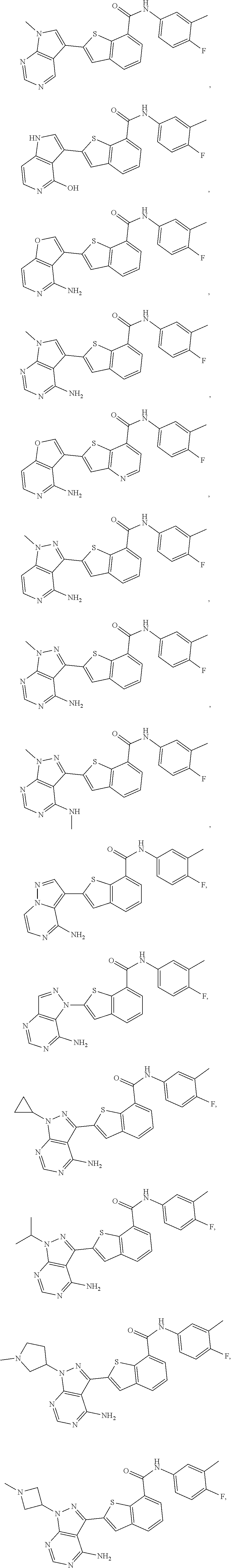

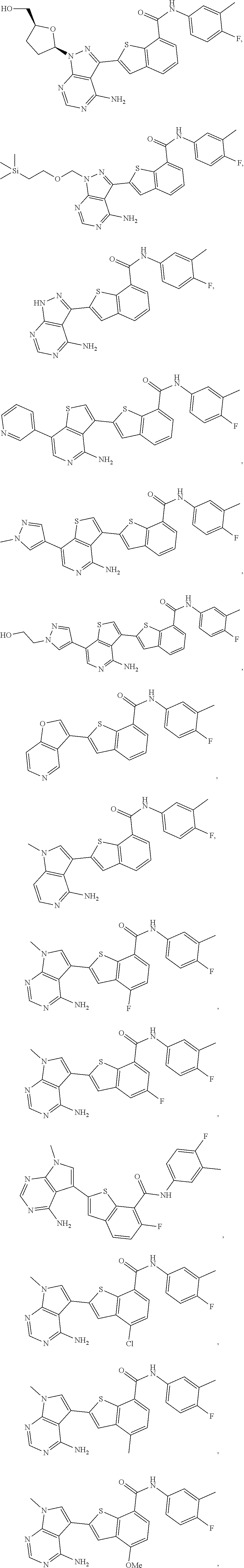

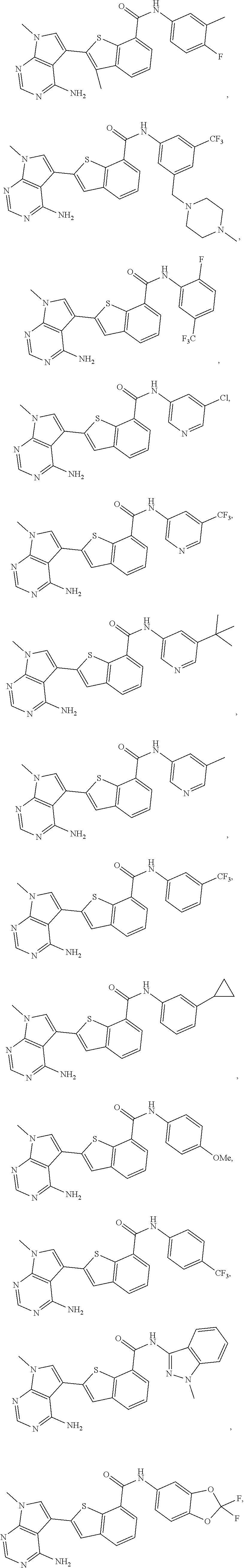

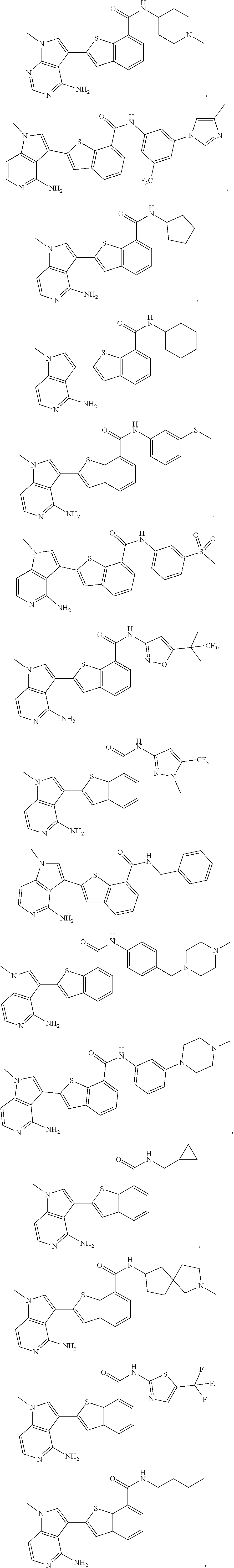

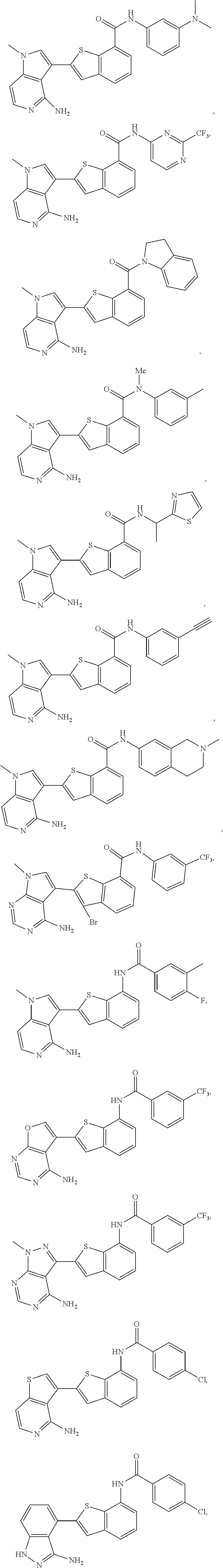

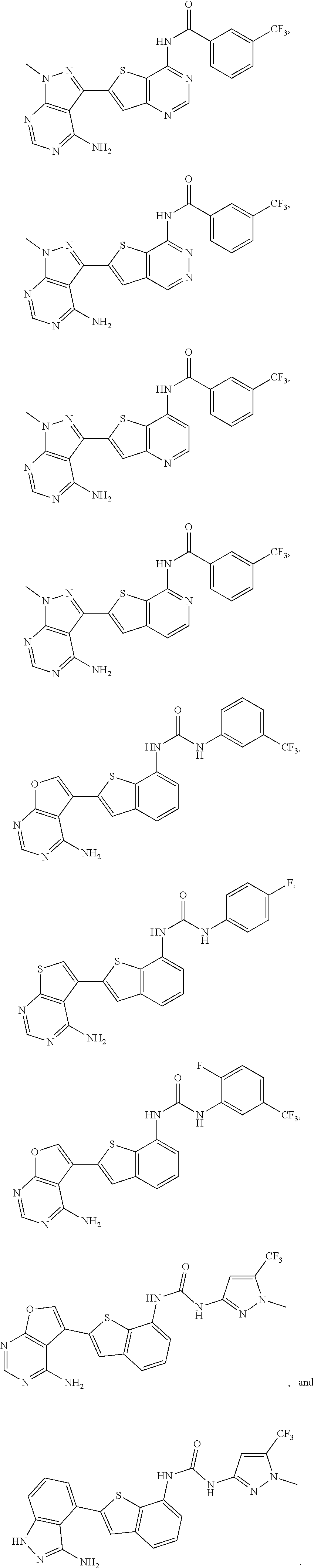

61. A pharmaceutical composition, wherein the pharmaceutical composition comprises the compound or the pharmaceutically acceptable salt of Formula III or the stereoisomer or isotopologue thereof according to claim 42 and one or more pharmaceutically acceptable excipients.

62. A pharmaceutical composition, wherein the pharmaceutical composition comprises the compound or the pharmaceutically acceptable salt thereof or the stereoisomer thereof according to claim 60 and one or more pharmaceutically acceptable excipients.
